(19) United States
(12) Patent Application Publication
(10) Pub. No.: US 2025/0123297 A1
(43) Pub. Date: Apr. 17, 2025

(54) Supermere Nanoparticles and Methods of Isolation and Use Thereof

(71) Applicant: Vanderbilt University, Nashville, TN (US)

(72) Inventors: Robert J. Coffey, Jr., Nashville, TN (US); Jeffrey Franklin, Nashville, TN (US); Dennis Jeppesen, Nashville, TN (US); James N. Higginbotham, Nashville, TN (US); Qin Zhang, Nashville, TN (US)

(21) Appl. No.: 18/695,574

(22) PCT Filed: Oct. 4, 2022
(86) PCT No.: PCT/US2022/077513

§ 371 Date: Mar. 26, 2024

Related U.S. Application Data
(60) Provisional application No. 63/253,945, filed on Oct. 8, 2021.

(51) Int. Cl. G01N 33/68

(52) U.S. Cl. CPC G01N 33/6896; G01N 2333/4709; G01N 2333/495; G01N 2333/902; G01N 2333/91205; G01N 2333/988; G01N 2333/99; G01N 2800/2821

(57) ABSTRACT

Disclosed herein is a newly identified secreted nanoparticle that is morphologically and molecularly distinct from the recently described nanoparticle termed an exomere. The disclosed nanoparticle is referred to herein as a supermere. Both exomeres and supermeres are amembranous in contrast to membrane-enclosed extracellular vesicles (EVs). Supermeres are smaller and morphologically distinct from exomeres. These supermeres contain cargo with diagnostic and therapeutic applications.

### CROSS-REFERENCE TO RELATED APPLICATIONS

This application claims benefit of U.S. Provisional Application No. 63/253,945, filed Oct. 8, 2021, which is hereby incorporated herein by reference in its entirety.

### STATEMENT REGARDING FEDERALLY SPONSORED RESEARCH OR DEVELOPMENT

This invention was made with Government Support under Grant No. CA197570, CA179514, and CA241685 awarded by the National Institutes of Health. The Government has certain rights in the invention.

### BACKGROUND

There is an increasing appreciation for the heterogeneous nature of secreted extracellular vesicles (EVs) and non-vesicular nanoparticles (Jeppesen, D. K., et al. Cell 2019 177:428-445 e418; Zhang, H., et al. Nat Cell Biol 2018 20:332-343; Zhang, Q., et al. Cell Rep 2019 27:940-954 e946). Exosomes are 40-150 nm endosome-derived, lipid-bilayer enclosed, small extracellular vesicless (sEVs) (Jeppesen, D. K., et al. Cell 2019 177:428-445 e418; Mathieu, M., et al. Nat Cell Biol 2019 21:9-17; van Niel, G., et al. Nat Rev Mol Cell Biol 2018 19:213-228). Recently, a new type of small (<50 nm), non-membranous, extracellular nanoparticle, termed the exomere, was identified (Zhang, H., et al. Nat Cell Biol 2018 20:332-343). Both exosomes and exomeres are released by most cells and tissues under both physiological and pathological conditions. Their production and content appear to be altered in a number of disease states, including neoplastic, cardiovascular, immunological and neurological disorders. However, intrinsic heterogeneity and variable methods of isolation pose major challenges to realizing their clinical potential.

### SUMMARY

Disclosed herein is a newly identified secreted nanoparticle that is morphologically and molecularly distinct from the recently described nanoparticle termed an exomere. The disclosed nanoparticle is referred to herein as a supermere. Both exomeres and supermeres are amembranous in contrast to membrane-enclosed extracellular vesicles (EVs). Supermeres are smaller and morphologically distinct from exomeres. These supermeres contain cargo that contribute to the pathogenesis of colorectal cancer (CRC) and may be potential biomarkers for CRC as some of these cargo have been detected in the circulation of cancer patients. In addition, supermeres from cetuximab-resistant CRC cells are able to confer resistance when added to cetuximab-sensitive CRC cells. Moreover, the receptor for SARS-CoV-2, angiotensin-converting enzyme 2 (ACE2), is present in large amounts in supermeres isolated from conditioned medium of DiFi cells, a human CRC cell line. The isoforms detected in these supermeres are smaller than the full-length band detected in DiFi whole cell lysates and in small EVs; this smaller form corresponds to the ectodomain of ACE2. Moreover, ACE2 is α2,6-sialylated in DiFi cells, small EVs, exomeres and supermeres. A processed form of amyloid precursor protein (APP), a protein that is associated with the pathogenesis of Alzheimer's disease, was also detected in supermeres, as well as the receptor tyrosine kinase MET and glypican-1 (GPC1).

Supermeres can be obtained relatively quickly and in large amounts from conditioned medium and plasma. They contain a number of cargo that may serve as biomarkers for a number of disease states, including cancer, heart disease, and neurodegenerative disorders, amongst others. Because these are new secreted components of biofluids, they have not thus far been associated with disease states.

Supermeres contain a large fragment of the ectodomain of ACE2. As disclosed herein, this form of ACE2 may bind SARS-CoV-2 and reduce the severity of infection, as has been shown for soluble ACE2. ACE2 is downregulated during viral infection, most notably with coronavirus infection (SARS and COVID-19), and this process can drive proteins towards secretion. In addition, ACE2 present in the circulation may be relevant to disorders of Renin-Angiotensin Aldosterone System (RAS/RAAS) that is associated with cardiovascular disease and cardiovascular problems associated with viral infection. Therefore, ACE2 in supermeres may provide diagnostic information. By delivering ACE2 in different forms in supermeres to patients systemically and/or in an aerosolized form might provide a specific treatment for patients. In addition, disclosed herein is a strategy of delivering this form of ACE2 in nanoparticles systemically and in an aerosolized form into the lungs.

Also disclosed herein is a method for isolating secreted, non-membranous supermere nanoparticles from a biological sample or conditioned medium, the method involving: centrifuging the biological sample or conditioned medium at 250 to 350×g (e.g. 300×g) to produce a first supernatant free of cell debris; filtering the first supernatant with a 0.22 μm filter to produce a first filtrate with reduced microparticle contamination; ultracentrifuging the first filtrate with a 100,000 molecular weight cutoff centrifugal concentrator to produce a first concentrate; ultracentrifuging the first concentrate at 100,000 to 167,000×g for 1 to 4 hours to produce a first pellet that is enriched for extracellular vesicles and exosomes; removing this first supernatant above the pellet; ultracentrifuging the supernatant at 100,000 to 167,000×g for 16 to 18 hours to produce a second pellet that comprises exomeres and a second supernatant from above the exomere pellet that contains supermeres; ultracentrifuging the second supernatant at 300,000 to 400,000 (e.g. 368,000×g) for 16 to 18 hours to produce a third pellet that comprises supermeres; resuspending the third pellet in a physiological solution (e.g. PBS-HEPES (PBS-H) (25 mM vol/vol).

Also disclosed is a method for diagnosis colorectal cancer in a subject that involves isolating a biological sample from the subject; isolating supermeres from the sample according to the disclosed method; and assaying the supermeres for colorectal cancer biomarker.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1C show Atomic Force Microscopy (AFM) imaging reveals morphological variations among nanoparticle fractions isolated from DiFi cells. FIG. 1A contains representative AFM topographic images of exosomes, non-vesicle, exomeres and supermeres. FIG. 1B is a box plot showing significantly higher mean±S.E.M particle size for exomeres (31.0±0.5 nm) compared to supermeres (23.3±0.34 nm) (P=1.4×10⁻²⁶) (n˜240 particles each). FIG. 1C shows cross sectional height profiles measured along the width of the particles show increased height for exomeres compared to supermeres. While exomeres show bulging topography, supermeres show indented topographies, as outlined by two black arrows marked in FIG. 1C.

FIGS. 2A and 2B show AFM particle size distributions and height variations among nanoparticle fractions isolated from DiFi cells. FIG. 2A contains histograms with Gaussian fits showing corresponding particle size distributions for exosomes, non-vesicle, exomeres and supermeres with means±S.E.M. FIG. 2C compares cross-sectional height profiles (measured along the width of the particles) (blue) of exomeres and supermeres (red). Height profiles for exosomes are shown in black for reference.

FIG. 3 shows extracellular presence of ACE2. FIG. 3 contains an immunoblot of ACE2 expression in DiFi cells, and pellet of small extracellular vesicles (sEV-P), exomeres and supermeres released from DiFi cells. The antibody recognizes an N-terminal (ectodomain) epitope of ACE2.

FIG. 4 shows ACE2 is α2,6-sialylated. Lysates from DiFi cells (WCL), small extracellular vesicle pellet (sEV-P), exomeres and supermeres derived from DiFi cells were incubated with agarose-conjugated Sambucus nigra agglutinin (SNA) lectin. (left) The total α2,6-sialylated proteins were precipitated followed by immunoblotting for the expression of ACE2 using an antibody recognizing an N-terminal (ectodomain) epitope. (right) Blot with lower exposure.

FIGS. 5A to 5H show supermeres are extracellular nanoparticles with distinct uptake in vitro and in vivo. FIG. 5A is a simplified schematic illustration of supermere isolation procedure. NV, non-vesicular fractions; sEV, small extracellular vesicles; sEV-P, small extracellular vesicle pellet. FIG. 5B contains representative fluid-phase AFM topographic images of sEVs, NVs, exomeres and supermeres derived from DiFi cells. Scale bar, 100 nm. FIG. 5C contains box plots of exomere and supermere heights and diameters measured by AFM (mean±s.e.m). n=10 for height and n=134 for diameter, **P<0.001 (two-tailed t-test). For boxplots the center lines mark the median; box limits indicate 25th and 75th percentiles; whiskers extend 1.5 times the interquartile range from 25th and 75th percentiles. FIG. 5D shows imaging of vesicle and particle uptake. MDA-MB-231 cells were incubated with PBS (CTL, control), or Alexa Fluor-647-labeled sEVs, exomeres or supermeres, and imaged every 15 min for 24 h with iSIM. For each field of view, the average fluorescence intensity was measured. Each field of view was averaged and normalized to the starting value. n=100. Scale bar, 10 μm. FIG. 5E shows inhibition of cellular supermere uptake. MDA-MB-231 or Hela cells were pre-incubated with uptake inhibitors for 30 min before addition of Alexa Fluor-647-labeled supermeres. After 24 h incubation, brightfield and fluorescence images were acquired with iSIM. ROIs were manually drawn based on brightfield images and then used to determine the mean fluorescence intensity of each cell. n=30 (MDA-MB-231) and n=27 (Hela), *P<0.01 (one-way ANOVA, Holm-Bonferroni correction) Representative data from 3 independent experiments are shown. Scale bar, 20 μm. FIG. 5F shows supermere co-localization with endo/lysosomal compartments following uptake. MDA-MB-231 cells were incubated with Alexa Fluor-647-labeled supermeres and stained with LysoTracker and images were acquired with iSIM. Scale bar, 5 μm (left) and 2 μm (right). FIG. 5G show whole organ imaging of small EV-Ps, exomeres and supermeres. Male C57BL/6 mice were injected via intraperitoneal injection with PBS (CTL, control), NIR dye-labeled small EVs, exomeres or supermeres derived from DiFi cells. Organs were harvested 24 h after injection and analyzed using an Odyssey FC imaging system. Data are mean±s.e.m. n=3, *P<0.01; **P<0.001 (two-tailed t-test). FIG. 5H shows immunoblot of select proteins in the sEV-P, exomeres and supermeres derived from Calu-3−, LIM1215−, DiFi 1342 cells, and a colorectal cancer (CRC) patient plasma sample. Thirty micrograms of protein from each fraction were analyzed. WCL, whole cell lysate; exom, exomere; super, supermere.

FIG. 6A to 6L shows supermeres exhibit distinct proteomic profiles and the supermere protein TGFBI is a potential CRC biomarker. FIG. 6A is a Venn diagram of unique and common proteins identified in DiFi-derived sEVs, NV, exomeres and supermeres. FIG. 6B shows principal component analysis of normalized DiFi proteomic mass spectral counts. FIG. 6C is a heatmap of top 20 most abundant proteins in all samples from DiFi. FIG. 6D is a heatmap of top 20 most abundant proteins in supermeres derived from DiFi, PANC-1 and MDA-MB-231 cells. FIG. 6E is a Venn diagram of unique and common top 50 most abundant proteins identified in supermeres derived from DiFi, PANC-1 and MDA-MB-231 cells. FIG. 6F is an immunoblot of representative proteins in DiFi, PANC-1 and MDA-MB-231-derived supermeres. Thirty micrograms of protein from each fraction were analyzed. FIG. 6G shows FAVS analysis of TGFBI level in the sEV-P, exomeres and supermeres derived from DiFi cells. FIG. 6H shows immunohistochemical staining of TGFBI expression in normal (NL) colon and colorectal cancer (CRC) tissue samples. Representative images are shown. Scale bar, 100 μm. FIGS. 6I and 6J shows overall survival (FIG. 6I) and progression-free survival (FIG. 6J) analysis of CRC patients comparing TGFBI level (high vs. low) using Kaplan and Meier method with data compared among marker groups using the log-rank test. FIG. 6K shows ELISA analysis of TGFBI levels in supermeres derived from normal or CRC patient plasma. FIG. 6L shows FAVS analysis of TGFBI level in sEV-P, exomeres and supermeres derived from CRC patient plasma.

FIGS. 7A to 7J show supermeres increase lactate release and transfer drug resistance. FIG. 7A is a heatmap of normalized spectral counts for select proteins and enzymes involved in glycolysis in sEVs, NV, exomeres (exom) and supermeres (super) from DiFi cells. FIG. 7B shows GSEA analysis of pathways enriched in metabolic enzymes for supermere vs sEV (top) and supermere vs exomere (bottom) from DiFi cells. FIGS. 7C and 7D shows immunoblot analysis of select metabolic enzymes and proteins involved in glycolysis in cells and extracellular samples derived from DiFi (FIG. 7C), PANC-1, SC, LM2-4175, MDAM-MB-231 and HREC (FIG. 7D) cells. Thirty micrograms of protein from each fraction were analyzed. FIG. 7E shows immunoblot analysis of ENO2 and LDHA in DiFi whole cell lysate as well as high-resolution density gradient-fractionated sEVs, NV, and exomeres and supermeres. Thirty micrograms of protein from each fraction were analyzed. FIG. 7F shows lactate release of CC cells treated with PBS (CTL, control) or 50 μg/ml supermeres derived from CC, SC or CC-CR cells is plotted as mean±s.e.m. n=3. *P<0.01. FIG. 7G shows CC colony growth analysis in 3D collagen treated with 50 μg/ml supermere derived from CC, SC or CC-CR in the presence or absence of cetuximab (CTX) for 14 days. Colony counts plotted as mean±s.e.m. n=9. *P<0.01; **P<0.001. FIG. 7H are representative images of CC colonies from (FIG. 7G). Scale bar, 200 μm. FIG. 7I are representative low (upper) and high (lower) magnification images of CC colonies treated with SC supermere. Scale bar, 200 μm. FIG. 7J shows DiFi colony growth analysis in 3D collagen treated with 50 μg/ml sEV-P, exomeres and supermeress derived from DiFi cells in the presence or absence of CTX for 14 days. Colony counts plotted as mean±s.e.m. n=6. *P<0.01.

FIGS. 8A to 8I show supermeres and exomeres are highly enriched with clinically relevant shed membrane proteins. FIG. 8A is a heatmap of normalized spectral counts of APP and other selected membrane proteins involved in Alzheimer's disease. FIG. 8B shows immunoblot analysis of APP in DiFi cells, the sEV-P, exomeres, and supermeres using N-terminal (N, left) and C-terminal (C, right) APP antibodies. (c), C-terminal fragment; Exomere, exom; supermere, super; (i), immature APP; (m), mature APP; (s), soluble APP. Thirty micrograms of protein from each fraction were analyzed. FIG. 8C shows FAVS analysis of APP in the DiFi sEV-P, exomeres and supermeres. FIG. 8D shows immunoblot analysis of MET in SC cells and corresponding extracellular samples using both N-terminal (N, left) and C-terminal (C, right) MET antibodies. (c), C-terminal MET fragment; (p), pro-form MET; (s), soluble MET. Thirty micrograms of protein from each fraction were analyzed. FIG. 8E shows FAVS analysis of MET in the DiFi sEV-P, exomeres and supermeres using MET antibody directly conjugated to Alexa-647. FIG. 8F shows immunoblot analysis of GPC1 in cells, the sEV-P, exomeres and supermeres derived from PANC-1 cells (left) and human renal epithelial cells (HREC, right) using a rabbit monoclonal antibody. Thirty micrograms of protein from each fraction were analyzed. FIG. 8G shows FAVS analysis of GPC1 in the DiFi sEV-P, exomeres and supermeres. FIG. 8H shows immunoblot analysis of CEA in cells, the sEV-P, exomeres and supermeres derived from DiFi, LS174T, LIM1215 and Calu-3 cells. FIG. 8I shows immunoblot analysis of CEA in the sEV-P, exomeres and supermeres isolated from normal (NL) and colorectal cancer (CRC) patient plasmas. Thirty micrograms of protein from each fraction were analyzed.

FIGS. 9A to 9M shows differential expression of small RNAs in sEVs and extracellular nanoparticles. FIG. 9A shows relative RNA abundance in the DiFi sEV-P, exomeres and supermeres. n=3 biological replicates. *P<0.05; **P<0.01. For boxplots the center lines mark the median; box limits indicate 25th and 75th percentiles; whiskers extend 1.5 times the interquartile range from 25th and 75th percentiles. FIG. 9B shows percentage of small RNA reads mapped small non-coding RNA for DiFi cells, the sEV-P, exomeres and supermeres following RNA-seq. n=3 biological replicates. FIG. 9C shows principal component analysis of normalized miRNA reads for DiFi cells, the sEV-P, exomeres and supermeres following RNA-seq. n=3 biological replicates. FIG. 9D is a heat map of top 25 most abundant miRNAs across DiFi cells and extracellular compartments. FIG. 9E shows qRT-PCR analysis of miR-1246 expression in DiFi cells and extracellular compartments. The mean Ct value for miR-1246 and U6 are displayed in the table. n=3. FIG. 9F shows representative FISH staining of miR-1246 in human normal colonic tissue (NL) and colorectal cancer (CRC) from a tissue microarray (TMA). Scale bar, 100 μm (left panel, low magnification) and 20 μm (right panel, high magnification). FIG. 9G shows percentage of normalized DiFi small RNA reads containing the miR-1246 sequence. Total normalized small RNA reads containing miR-1246 reads are displayed in the table. FIGS. 9H to 9K is an immunoblot of representative RNA binding proteins identified in extracellular compartments derived from DiFi (FIGS. 9H, 9I), PANC-1 (FIG. 9J) and SC cells (FIG. 9K). WCL, whole cell lysate; sEV-P, small extracellular vesicle pellet; sEV, small extracellular vesicle; NV, non-vesicular; exom, exomere; super, supermere. Thirty micrograms of protein from each fraction were analyzed. FIG. 9L shows immunohistochemical staining of AGO2 expression in adjacent normal colon and CRC samples. Representative images are shown. Scale bar, 100 μm. FIG. 9M shows FAVS analysis of AGO2 levels from the plasma of normal controls and CRC patients. Representative results are shown. n=3.

FIGS. 10A to 10K show supermere effects on liver in vivo. FIG. 10A is a schematic of mouse treatment experiments. FIG. 10B shows liver to body weight ratio of mice following PBS (CTL, control), exomere or supermere treatments. *P<0.05 (Kruskal-Wallis, post hoc Dunn's). n=5-6. FIG. 10C shows Oil Red-O staining of mouse livers following three consecutive injections with PBS (CTL, control), exomeres or supermeres derived from DiFi cells. Livers were harvested 24 h post last injection. Oil Red-O staining shows dose-dependent decreased staining with exomeres and supermeres. Scale bar, 20 μm. CV, central vein. FIG. 10D shows level of triglycerides in liver tissue following injection with exomeres or supermeres derived from DiFi cells. Triglyceride concentration shows dose-dependent decrease after injection of exomeres and supermeres. *P<0.05 (Wilcoxon rank sum). n=5-6. For boxplots the center lines mark the median; box limits indicate 25th and 75th percentiles; whiskers extend 1.5 times the interquartile range from 25th and 75th percentiles. FIG. 10E shows periodic acid-Schiff (PAS) staining of formalin-fixed, paraffin-embedded (FFPE) liver tissue following injection with exomeres or supermeres derived from DiFi cells. There were significant differences between experimental groups by pathology scoring of hepatocytes containing darker magenta deposits of polysaccharides (arrowhead, P=0.038, Kruskal-Wallis). The most significant reduction was noted between supermere groups and PBS (CTL, control). *P<0.05 (Wilcoxon rank sum). Representative images are shown. CV, centrilobular vein. Enlarged inset diameter, approximately 90 μm. Scale bar, 100 μm. FIG. 10F shows histological scoring of liver sections stained with PAS. Sections were scored double-blinded (0-3) for intensity and homogeneity by two liver pathologists. Liver sections from mice injected with 300 μg of supermeres showed decreased score compare to other treatment groups. n=5-6. *P<0.05 (Wilcoxon rank sum). FIG. 10G is an immunoblot of select proteins in mouse liver lysates after treatment with PBS (control), 300 μg of exomeres or supermeres. Thirty micrograms of protein from each treatment group were analyzed. FIG. 10H shows quantification of proteins detected by immunoblot. Error bars represent mean±s.e.m. n=3. Statistical significance corrected by Holm-Bonferroni, *P<0.05. FIG. 10I shows select GSEA pathways significantly down- or upregulated in mouse liver cells compared to PBS following treatment with exomeres or supermeres. FIG. 10J is a Venn diagram of unique and common differentially expressed genes, compared to control (PBS), between exomere and supermere treated mice. The criteria for inclusion of a differentially expressed gene was fold-change >1.5 and FDR <1.0. FIG. 10K shows principal component analysis of mouse liver cell gene expression following treatment.

FIGS. 11A to 11L show DPEP1 in exosomes and FASN in exomeres are potential CRC biomarkers, and CD73 is a general marker for exosomes. FIG. 11A is an immunoblot of representative proteins identified in DiFi cells (WCL), sEVs, NV and exomeres (exom). Thirty micrograms of protein from each fraction were analyzed. FIG. 11B is an immunoblot of representative proteins identified in sEVs sorted by FAVS based on expression of EGFR and CD81. The same number of sorted vesicles (1.5×10⁶) for each sample were analyzed. FIG. 11C shows localization of endogenous CD63 and DPEP1 in DiFi cells imaged with 3D SIM. Left: 1.8 μm z-stack projection. Scale bar, 5 μm. Right: enlarged inserts. Scale bar, 500 nm. FIG. 11D shows level of α2,6-sialylated DPEP1 and CD73 detected in DiFi cells, the sEV-P, exomeres and supermeres (super). FIG. 11E shows immunohistochemical staining of DPEP1 expression in normal colon (NL) and colorectal cancer (CRC) tissue samples. Representative images are shown. Scale bar, 100 μm. FIG. 11F shows overall survival analysis of CRC patients comparing DPEP1 staining pattern (diffuse vs. others) using Kaplan and Meier, and data compared among marker groups using log-rank test. FIG. 11G shows FAVS analysis of DPEP1 and CEA levels in the sEV-P from the plasma of normal controls and CRC patients using DPEP1 antibody directly conjugated to phycoerythrin (PE). Blue boxes indicate DPEP1-positive sEVs and red boxes indicate DPEP1- and CEA-double positive sEVs. FIG. 11H shows immunoblot analysis of CD73 expression in cells (WCL), the sEV-P and exomeres (exom) from DKO-1, LS174T, MDA-MB-231, LM2-4175, PANC-1, Gli36vIII, Calu-3 and HREC cells. Thirty micrograms of protein from each fraction were analyzed. FIG. 11I shows immunohistochemical staining of CD73 expression in normal colon and CRC tissue samples. Representative low (left) and high (right) magnification images are shown. Scale bar, 100 μm. FIG. 11J shows immunoblot analysis of CD73 in the sEV-P and exomeres isolated from normal and CRC patient plasma samples. FIG. 11K shows immunohistochemical staining of FASN expression in adjacent normal colon and CRC tissue samples. Representative images are shown. Scale bar, 100 μm. FIG. 11L shows FAVS analysis of FASN levels in the sEV-P and exomeres of plasma from normal controls and CRC patients using FASN antibody directly conjugated to Alexa-647. Representative data are shown.

FIGS. 12A to 12G shows supermeres are extracellular particles with distinct uptake in vitro. FIG. 12A is a schematic of the isolation procedure for the sEV-P, sEVs, NV, exomeres and supermeres. FIG. 12B shows negative stain transmission electron microscopy of DiFi-derived sEVs, NV, exomeres and supermeres. FIG. 12C shows negative stain transmission electron microscopy of SC-derived exomeres and supermeres. FIG. 12D shows representative fluid-phase AFM topographic images of exomeres and supermeres derived from MDA-MB-231 cells. Scale bar, 100 nm (left). Box plots of exomere and supermere diameters measured by AFM. n=108, **P<0.001 (two-tailed t-test). For boxplots the center lines mark the median; box limits indicate 25th and 75th percentiles; whiskers extend 1.5 times the interquartile range from 25th and 75th percentiles (right). FIG. 12E shows cellular imaging of vesicle and particle uptake. MDA-MB-231 cells were incubated with Alexa Fluor-647-labeled sEVs, exomeres or supermeres, and imaged every 15 min for 24 h with iSIM. For each field of view, the average fluorescence intensity was measured. Each field of view was averaged and normalized to the starting value. n=100. Scalebar, 10 μm. FIG. 12F shows inhibition of cellular supermere uptake. MDA-MB-231 or Hela cells were pre-incubated with indicated uptake inhibitors for 30 min before addition of Alexa Fluor-647-labeled supermeres. After 24 h incubation, brightfield and fluorescence images were acquired with iSIM. Scalebar, 20 μm. FIG. 12G shows immunoblot of selected proteins in Calu-3-, LIM1215-, and PANC-1-derived sEV-Ps, exomeres (exom) and supermeres (super). Thirty micrograms of protein from each fraction were analyzed. WCL, whole cell lysate. I.e, lower exposure; g.e, greater exposure.

FIGS. 13A to 13H show supermeres exhibit distinct proteomic profiles. FIG. 13A is a heatmap of top 25 differentially expressed proteins in sEVs, NV, exomeres and supermeres from DiFi cells, based on normalized spectral counts. FIGS. 13B and 13C are heatmaps of the relative abundance of select conventional sEV markers (FIG. 13B) and vacuolar protein sorting proteins (VPS) in sEVs, NV, exomeres and supermeres from DiFi cells (FIG. 13C). FIG. 13D shows immunoblot analysis of VPS35 in DiFi cells (WCL), the sEV-P, exomeres (exom) and supermeres (super). FIG. 13E shows protein concentrations and ratios of the sEV-P, exomeres and supermeres produced from cell lines in equal volumes. Note that the size of the sample preparations (number of cell culture plates) is not equal between different cell lines. FIG. 13F shows immunoblot analysis of SC and HREC cells (WCL), the sEV-P, exomeres and supermeres. FIG. 13G shows ELISA analysis of TGFBI levels in DiFi, PANC-1 and MDA-MB-231 cells, the sEV-P, exomeres and supermere. Data are mean±s.e.m. n=8 for DiFi, and n=3 for PANC-1 and MDA-MB-231. FIG. 13H shows immunohistochemical staining of TGFBI expression in normal colon (NL) and colorectal cancer (CRC) tissue samples. Representative images are shown. Scale bar, 100 μm.

FIGS. 14A to 14H show supermeres increase lactate release and transfer drug resistance. FIG. 14A shows lactate release from CC cells treated with PBS (CTL), or 5 or 50 μg/ml of the sEV-P or exomeres derived from CC, SC or CC-CR cells is plotted. n=3. FIG. 14B show GSEA analysis of pathways enriched in metabolic enzymes for supermeres vs sEVs (top) and supermeres vs exomeres (bottom) from DiFi cells. FIG. 14C shows CC colony growth analysis in 3D collagen treated with 50 μg/ml of CC or CC-CR-derived sEV-P or exomeres in the presence or absence of cetuximab (CTX) for 14 days. Colony counts are plotted (mean±s.e.m). n=9. FIG. 14D show CC colony growth analysis in 3D collagen treated with 5 or 50 μg/ml of the sEV-P or exomeres derived from CC, SC, or CC-CR cells in the presence or absence of CTX for 14 days. Colony counts are plotted (mean±s.e.m). n=3. FIG. 14E shows DiFi colony growth analysis in 3D collagen treated with 25 μg/ml of supermeres derived from SC cells in the presence or absence of CTX for 14 days. Colony counts are plotted (mean±s.e.m). n=3. FIG. 14F contains representative images of DiFi colonies from FIG. 14E. Scale bar, 200 μm. FIG. 14G shows CC colony growth analysis in 3D collagen treated with 25 μg/ml of supermeres derived from DiFi cells in the presence or absence of CTX for 14 days. Colony counts are plotted (mean±s.e.m). n=9. *P<0.01. FIG. 14H shows representative images of DiFi colonies from FIG. 7J. Scale bar, 200 μm.

FIGS. 15A to 151 show supermeres and exomeres are highly enriched with clinically relevant shed membrane proteins. FIG. 15A shows immunoblot analysis of APP in SC cells, the sEV-P, exomeres (exom) and supermeres (super) using N-terminal (N, left) and C-terminal (C, right) APP antibodies. (i), immature APP; (m), mature APP; (s), soluble APP. FIG. 15B shows immunoblot analysis of MET in DiFi cells, the sEV-P, exomeres and supermeres, using N-terminal (N, left) and C-terminal (C, right) MET antibodies. (c), C-terminal fragment MET; (p), pro-form MET; (s), soluble MET. FIG. 15C shows immunoblot analysis of EGFR in DiFi cells, the sEV-P, exomeres and supermeres, using N-terminal (N) and C-terminal (C) EGFR antibodies. m, membrane; s, soluble. FIG. 15D shows immunoblot analysis of AREG in MDA-NB-231 cells and the sEV-P, exomeres and supermeres (left), and in CC cells and the sEV-P, exomeres and supermeres with short (left) and long exposure (right), using an N-terminal AREG antibody. g.e, greater exposure; I.e, lower exposure. FIG. 15E shows immunoblot analysis of GPC1 in Calu-3 cells, the sEV-P, exomeres and supermeres using a rabbit monoclonal GPC1 antibody. FIG. 15F shows immunoblot analysis of GPC1 in DiFi, SC, MDA-MB-231 and PANC-1 cells, and the sEV-P, exomere and supermere fractions, using a rabbit monoclonal GPC1 antibody. The PANC-1 immunoblot is a longer exposure of the corresponding membrane in FIG. 8F. FIG. 15G shows immunoblot analysis of GPC1 in PANC-1 cells, and the sEV-P, exomeres and supermeres, using a rabbit polyclonal GPC1 antibody with short (upper) and long exposure (lower) of the immunoblot. FIGS. 15H and 151 show MET sequence in DiFi-derived sEV and supermere identified by mass spectrometry.

FIGS. 16A to 16M show characterization of small RNAs associated with different fractions. FIG. 16A show bioanalyzer size profile of RNAs isolated from DiFi cells, the sEV-P, exomeres and supermeres. FIG. 16B shows principal component analysis of tRNAs in DiFi cells, the sEV-P, exomeres and supermeres. FIG. 16C is a heatmap of tRNA analysis in DiFi cells, the sEV-P, exomeres and supermeres. FIG. 16D is a heatmap of miRNAs analysis in DiFi cells, the sEV-P, exomeres and supermeres. FIG. 16E is a Venn diagram of miRNAs identified in DiFi cells, the sEV-P, exomeres and supermeres. n=3 biological replicates. FIG. 16F is a heatmap of top 10 differentially expressed miRNAs. Scale bar indicates intensity. DESeq2 was used to detect differential expression among samples. FIG. 16G is a heatmap of top 5 differentially expressed miRNAs in DiFi exomeres and supermeres. Scale bar indicates intensity.

FIG. 16H shows qRT-PCR analysis of miR-675-5p expression in DiFi cells, the sEV-P, exomeres and supermeres relative to U6. The mean Ct value for miR-675-5p and U6 are displayed in the table. n=3. FIG. 16I shows representative FISH staining of positive control of U6 (green) and negative control (CTL) of scrambled miRNA (green) in human normal tissue (NL) and colorectal cancer (CRC) tumors on a tissue microarray with DAPI (blue). Scale bars, 100 μm (left) and 20 μm (right). FIG. 16J shows percentage of normalized small RNA reads containing the miR-1246 sequence in cells, sEVs and NV fraction derived from DKO-1 and Gli36 vIII cells (data set from Jeppesen et al. 2019, https://www.cell.com/cell/article/S0092-8674(19)30212-0/fulltext). FIG. 16K is an immunoblot analysis of AGO1 and AGO2 expression in DiFi cells, sEVs, NV and exomeres. WCL, whole cell lysate; sEV, small extracellular vesicle; NV, non-vesicular; exom, exomere. FIG. 16L shows FAVS analysis of AGO2 expression in the DiFi sEV-P, exomeres and supermeres. FIG. 16M shows immunoblot analysis of AGO2 expression in LS174T cells, sEVs, NV and exomeres.

FIGS. 17A to 17D show organ biodistribution of supermeres and effects on liver in vivo. FIG. 17A shows histological scoring of liver sections stained with Red Oil O. Significance assessed by Wilcoxon rank sum. n=5-6. For boxplots the center lines mark the median; box limits indicate 25th and 75th percentiles; whiskers extend 1.5 times the interquartile range from 25th and 75th percentiles. CTL, control. FIG. 17B shows Periodic acid-Schiff (PAS) staining of formalin-fixed, paraffin-embedded (FFPE) liver tissue with or without glycogen digestion by diastase. CV, centrilobular vein. Scale bar, 100 μm. FIG. 17C shows hematoxylin and eosin (H&E) staining of FFPE liver tissue following injection with exomeres or supermeres derived from DiFi cells. PBS-injected control mice showed larger areas of enlarged hepatocytes with vacuolated cytoplasm, which extended to the edge of the CV. Exomere and supermere injected mice had a reduction of these large hepatocytes in the centrilobular area as delimited by the hyphenated line, when compared to the PBS control groups (Kruskal-Wallis, P=0.01). Enlarged inset diameter, approximately 68 μm. Scale bar, 75 μm. FIG. 17D shows histological scoring of liver H&E sections for the percentage of enlarged hepatocytes. n=5-6. The most significant reduction was between the supermere 300 μg group and the PBS controls. *P<0.05 (Wilcoxon rank sum).

FIGS. 18A to 18J show DPEP1 in exosomes and FASN in exomeres are potential CRC biomarkers. FIGS. 18A and 18B show immunoblot of DPEP1 expression in DiFi (FIG. 18A) and LS174T (FIG. 18A) cells (WCL), the sEV-P and exomeres (exom). FIG. 18C shows localization of endogenous CD63 and DPEP1 in DiFi cells imaged with confocal microscopy. Left panels: primary+secondary antibodies. Right panels: secondary antibodies only control. Scale bar, 10 μm. FIG. 18D shows DPEP1 expression from microarray platform U133 plus 2.0. FIG. 18E shows DPEP1 expression from TCGA RNAseq. FIG. 18F shows progression free survival analysis of CRC patients comparing DPEP1 staining pattern (diffuse vs. others) using Kaplan and Meier, and data compared among marker groups using log-rank test. FIG. 18G shows immunoblot analysis of FASN expression in cells, the sEV-P, exomeres and supermeres (super) from DiFi, SC, PANC-1, MDA-MB-231, LM2-4175 and HREC cells. FIG. 18H shows immunohistochemical staining of FASN expression in adjacent normal (NL) and cancer tissue samples of breast and prostate. Scale bar, 100 μm. FIG. 18I shows FAVS analysis of FASN level in the DiFi-derived sEV-P and exomeres using an FASN antibody directly conjugated to Alexa 647. FIG. 18J shows immunoblot analysis of ACLY expression in cells, the sEV-P and exomeres from LM2-4175 (top) and PANC-1 (bottom) cells.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Supermeres

The small (<50 nm), non-membranous nanoparticle, termed exomere, was recently identified by asymmetric flow field-flow fractionation (AF4) (Zhang et al., Nat. Cell Biol. 2018 20:332-343), which is incorporated by reference for the description of these isolation methods. An alternative method for isolating exomeres was described (Zhang et al. 2019. Cell Reports 27:940-954), which is incorporated by reference for the description of these isolation methods. Exomeres are highly enriched in metabolic enzymes and signature proteins involved in glycolysis and mTORC1 signaling (Zhang et al., 2018a). In addition to proteins, nucleic acids and lipids are also selectively secreted in exomeres.

Disclosed herein are a newly identified secreted, non-membranous nanoparticle, referred to herein as a “supermere,” which can be isolated from the supernatant used to produce exomeres.

Serum-free conditioned medium is removed and centrifuged for 15 minutes at 300×g to remove cellular debris. The resulting supernatant is then filtered through a 0.22-mm polyethersulfone filter (Nalgene, Rochester, NY) to reduce microparticle contamination. The filtrate is concentrated with a 100,000 molecular-weight cutoff centrifugal concentrator (Millipore). The concentrate is subjected to high-speed centrifugation at 167,000×g for 4 hours, and the resulting small EV (sEV)-enriched pellet is resuspended in PBS (e.g. containing 25 mM HEPES (pH 7.2)-PBSH) and washed. The remaining supernatant is subjected to centrifugation again at 167,000×g for at least 16 hours and not more than 18 hours; the resulting pellet is washed with fresh PBSH. This washed pellet contains exomeres. To isolate supermeres, the supernatant collected from the previous 16 to 18 hour ultracentrifugation step is ultracentrifuged at 368,000×g for at least 16 hours but not more than 18 hours. The resulting pellet is resuspended in PBS (e.g. containing 25 mM HEPES [pH 7.2]) and is designated supermeres.

Supermeres can therefore be isolated by first sequentially isolating EV-enriched material, next exomeres from cell-conditioned medium as previously described, or alternatively from plasma or other human or animal body fluids. The supernatant left over from the pelleting of exomeres can then be collected and subjected to ultracentrifugation at 368,000×g (rmax, 55000 RPM), e.g. using a Beckman Coulter SW55 Ti rotor for 16-18 hours. The resulting pellet represents supermeres. The supermeres can then be resuspended in a suitable liquid, such as PBS, and stored at 4° C.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Materials and Methods

Isolation of Supermeres: Exomeres were isolated from DiFi conditioned medium as previously described (Zhang et al. 2019. Cell Reports). The supernatant left over from the pelleting of exomeres was collected and subjected to ultracentrifugation at 368,000×g (rmax, 55000 RPM) using a Beckman Coulter SW55 Ti rotor for 18-24 hours. The resulting pellet contained the isolated supermeres. The supermeres were resuspended in PBS and stored 4° C. for downstream analysis and functional assays, or for some analyses, immediately resuspended in lysis buffers.

AFM imaging and analysis: Five micro-liters of freshly isolated EV samples were incubated on freshly cleaved mica substrates (TedPella Inc, CA) for 5 min, washed with molecular grade de-ionized water to remove any unbound EV and air-dried overnight. Samples were imaged by Dimension Icon (Bruker Instruments, CA, USA) using TESP probes (Bruker Instruments, CA, USA) and images were recorded in amplitude mode-AFM at 1024 samples per line at 1 Hz as described previously (ACS Nano. 2010 Apr. 27; 4(4): 1921-1926; ACS Nano. 2019, 13, 9, 10499-10511). Over six different scan regions were randomly selected for imaging of particles. Images were processed for zero order plane flattening using SPIP™ software (Denmark). The surface height was determined using histogram function and was subtracted from the measured height of particles. The particles were then individually detected and sizes were quantified using grain analysis function. All samples were stored at 4° C. and imaged within one week of isolation.

Statistical methods All statistical analyses were performed using commercially available OriginLab 9.1 or R software. All values are expressed as the mean±SEM. Differences in means between two groups were analyzed using unpaired two-sided heteroscedastic t-tests with Welch's correction. The level of significance was taken as p <0.05.

### Results

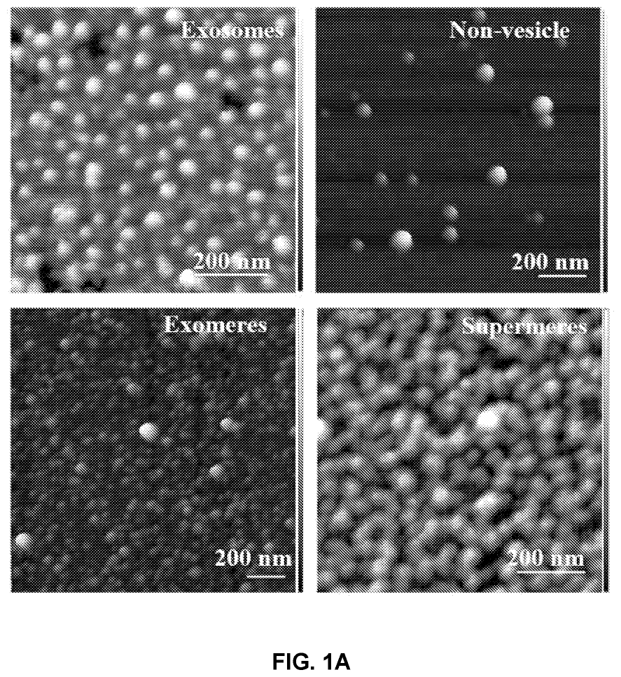

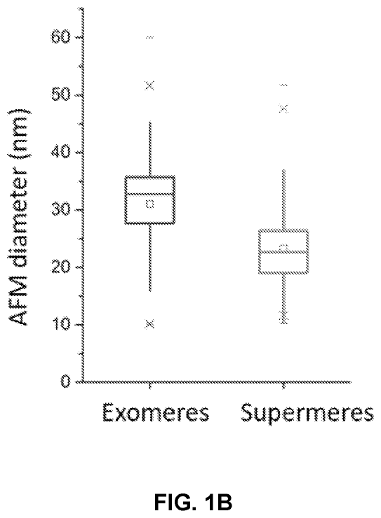

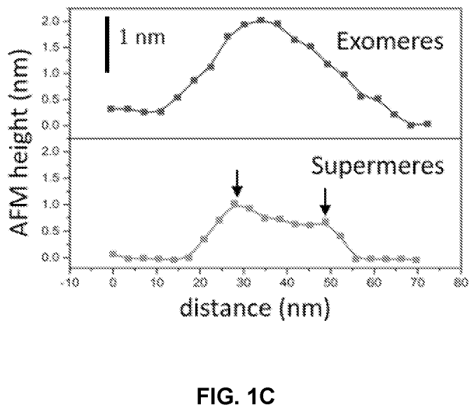

FIGS. 1A to 1C show AFM imaging reveals structural variations among nanoparticle fractions isolated from DiFi cells. FIG. 1A contains representative AFM topographic images of exosomes, non-vesicle fractions, exomeres and supermeres. FIG. 1B is a box plot showing significantly higher mean±S.E.M particle size for exomeres (31.0±0.5 nm) compared to supermeres (23.3±0.34 nm) (P=1.4×10⁻²⁶) (n˜240 particles each). FIG. 1C shows cross sectional height profiles measured along the width of the particles show increased height for exomeres compared to supermeres. While exomeres show bulging topography, supermeres show indented topographies, as outlined by two black arrows marked in FIG. 1C.

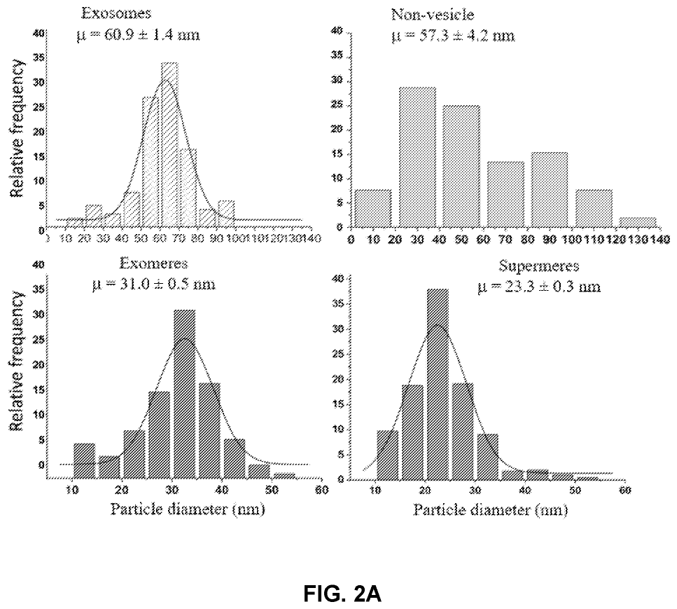

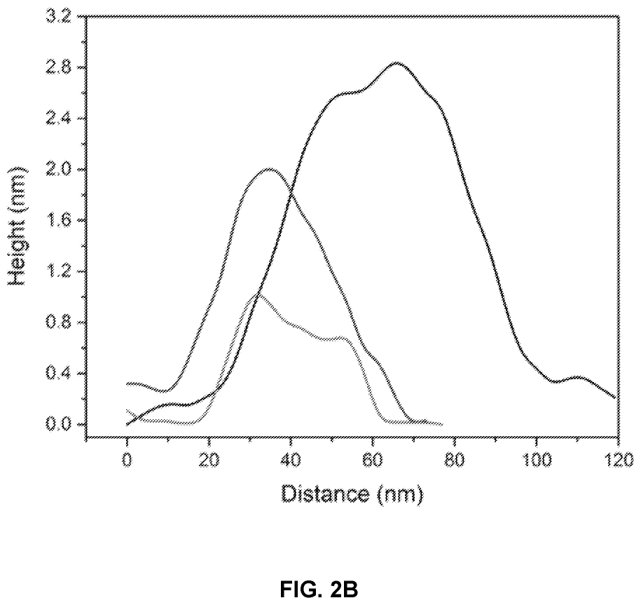

FIGS. 2A and 2B show AFM particle size distributions and height variations among nanoparticle fractions isolated from DiFi cells. FIG. 2A contains histograms with Gaussian fits showing corresponding particle size distributions for exosomes, non-vesicle, exomeres and supermeres with means±S.E.M. FIG. 2C shows cross sectional height profiles measured along the width of the particles show increased height for exomeres (blue) compared to supermeres (red). Height profiles for exosomes are shown in black for reference.

### Example 2

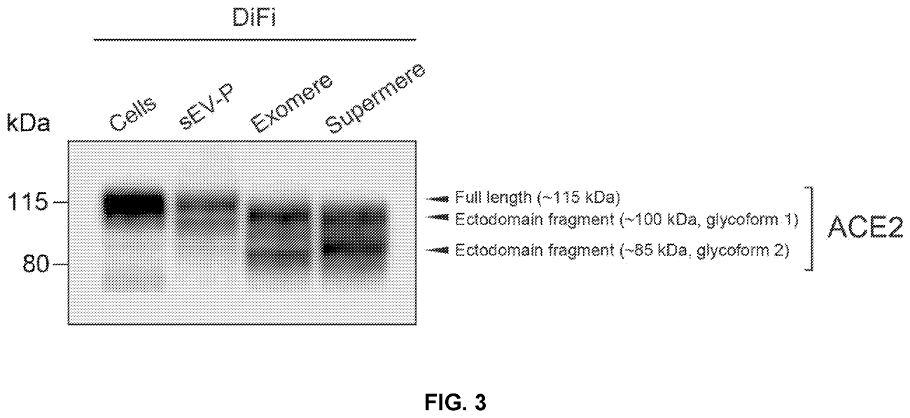

FIG. 3 shows extracellular presence of ACE2. FIG. 3 contains an immunoblot of ACE2 expression in DiFi cells, and pellet of small extracellular vesicles (sEV-P), exomeres and supermeres released from DiFi cells. The antibody recognizes an N-terminal (ectodomain) epitope of ACE2.

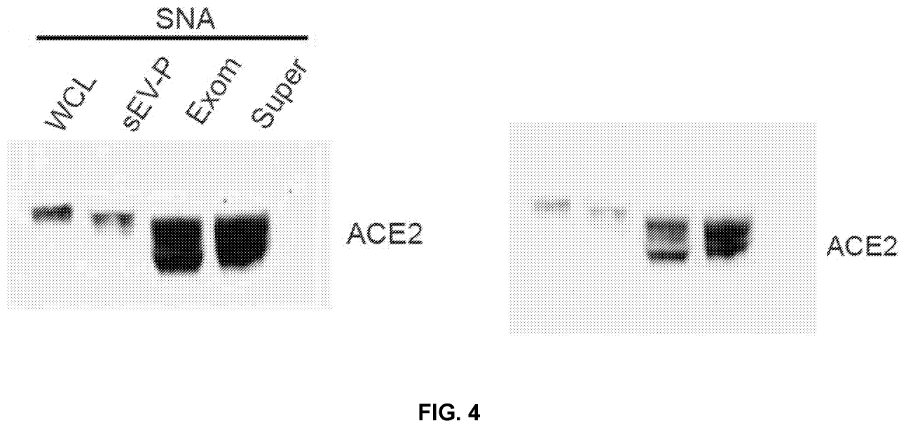

FIG. 4 shows ACE2 is α2,6-sialylated. Lysates from DiFi cells (WCL), small extracellular vesicle pellet (sEV-P), exomeres and supermeres derived from DiFi cells were incubated with agarose-conjugated Sambucus nigra agglutinin (SNA) lectin. (left) The total α2,6-sialylated proteins were precipitated followed by immunoblotting for the expression of ACE2 using an antibody recognizing an N-terminal (ectodomain) epitope. (right) Blot with lower exposure.

### Example 3

### Introduction

The present studies were initially designed to provide a comprehensive proteomic and RNA analysis of clinically relevant cargo unique to exosomes and exomeres in a human colorectal cancer (CRC) cell line, DiFi, using an optimized strategy to purify exosomes (Jeppesen, D. K., et al. Cell 2019 177:428-445 e418) and a simplified method to isolate exomeres (Zhang, Q., et al. Cell Rep 2019 27:940-954 e946). High-speed ultracentrifugation of the sEV supernatant resulted in isolation of amembranous nanoparticles identical in morphology and content to that reported in the original characterization of exomeres using asymmetric flow field-flow fractionation (AF4) (Zhang, H., et al. Nat Cell Biol 2018 20:332-343). Early on in the study, it was speculated that an additional population of nanoparticles might be identified in the supernatant after exomere purification by high-speed ultracentrifugation. In fact, a morphologically distinct new nanoparticle was discovered that is termed the supermere (supernatant of exomeres).

Supermeres are morphologically and structurally distinct from exomeres and display different cellular uptake kinetics than sEVs and exomeres in vitro. There is a greater uptake of supermeres in most tissues in vivo compared to sEVs and exomeres, especially in the brain compared to the other fractions. Supermeres contain the majority of extracellular small RNA. Many of the clinically relevant proteins and extracellular RNA (exRNA), previously reported to be in exosomes (amyloid precursor protein [APP], MET, glypican-1 [GPC1], argonaute-2 [AGO2)], miR-1246, TGFβ-induced [TGFBI] and numerous glycolytic enzymes) are highly enriched in supermeres. Three functional properties of supermeres were identified: increased lactate secretion in recipient cells (a hallmark of the Warburg effect), transfer of cetuximab resistance, and altered liver metabolism following systemic injection. Supermeres are detectable by optimized flow cytometry, opening up another potential avenue of investigation in liquid biopsies alongside circulating tumor cells, DNA and EVs.

The most abundant protein in highly purified DiFi exosomes was the glycophosphatidylinositol (GPI)-linked dipeptidase DPEP1 that has been linked to CRC metastasis (Park, S. Y., et al. Oncotarget 2016 7:9501-9512). Diffuse overexpression of DPEP1 in a clinically well-annotated CRC tissue microarray portends a worse outcome, and DPEP1 is increased in sEVs isolated from the plasma of CRC patients compared to normal individuals. Moreover, a subpopulation of exosomes released from DiFi cells is highly enriched in DPEP1, two circulating biomarkers for CRC (CEA and EPCAM), and the ectonucleotidase CD73 that is involved in immunosuppression and is overexpressed in CRC (Goswami, S., et al. Nat Med 2020 26:39-46; Hammami, A., et al. Semin Immunol 2019 42:101304).

Taken together, this work identifies a new functional extracellular nanoparticle that is morphologically and molecularly distinct from exosomes and is replete with potential biomarkers and targets for drug discovery. Moreover, the ability to isolate and inventory the contents of distinct populations of sEVs and nanoparticles is demonstrated so as to assign cargo to their correct carriers. These findings have potential important implications for cancer, Alzheimer's disease, heart disease, and COVID-19 infection.

Supermeres are Novel Extracellular Nanoparticles with Distinct Uptake In Vitro and In Vivo

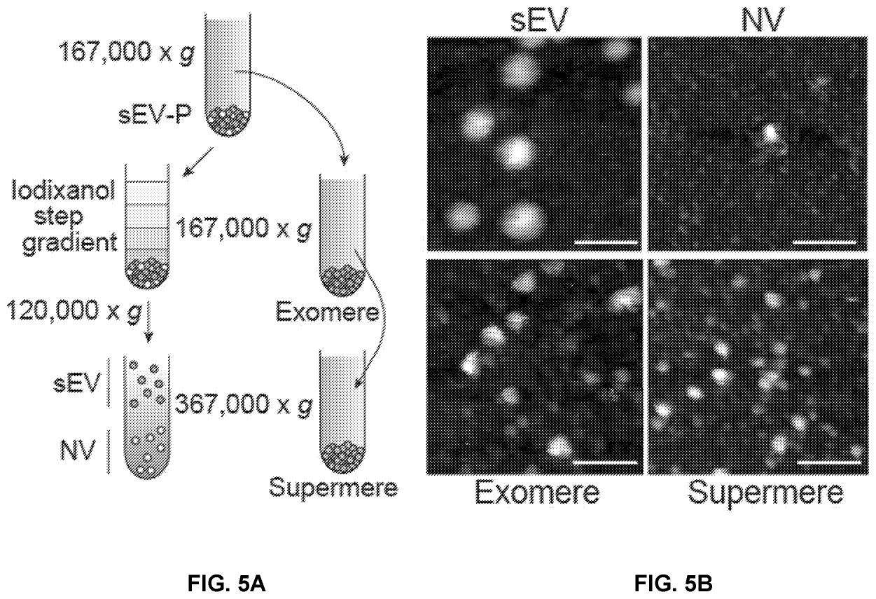

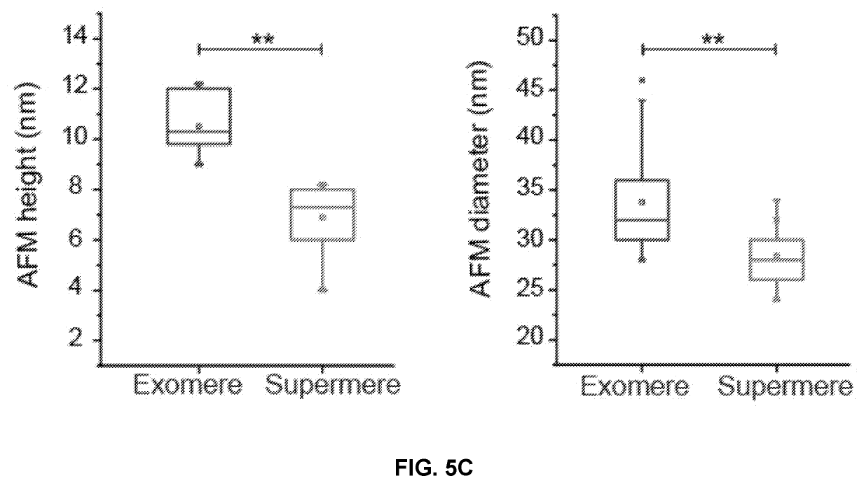

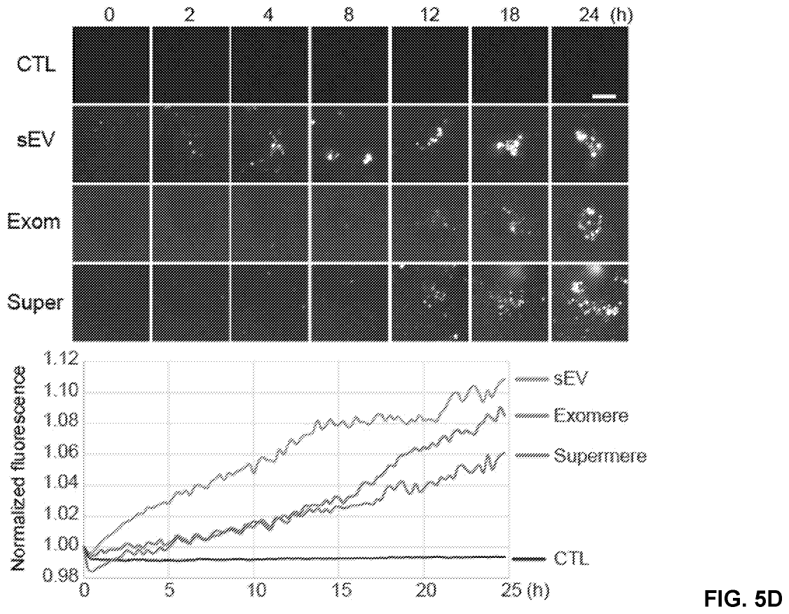

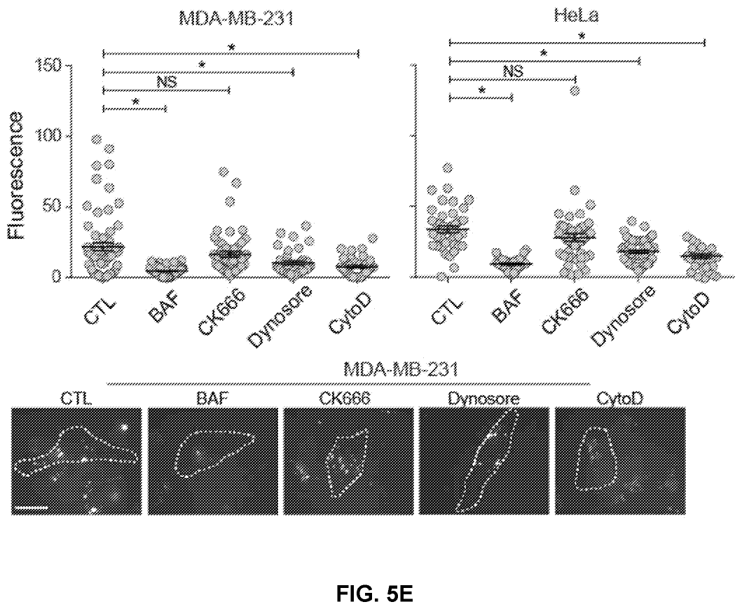

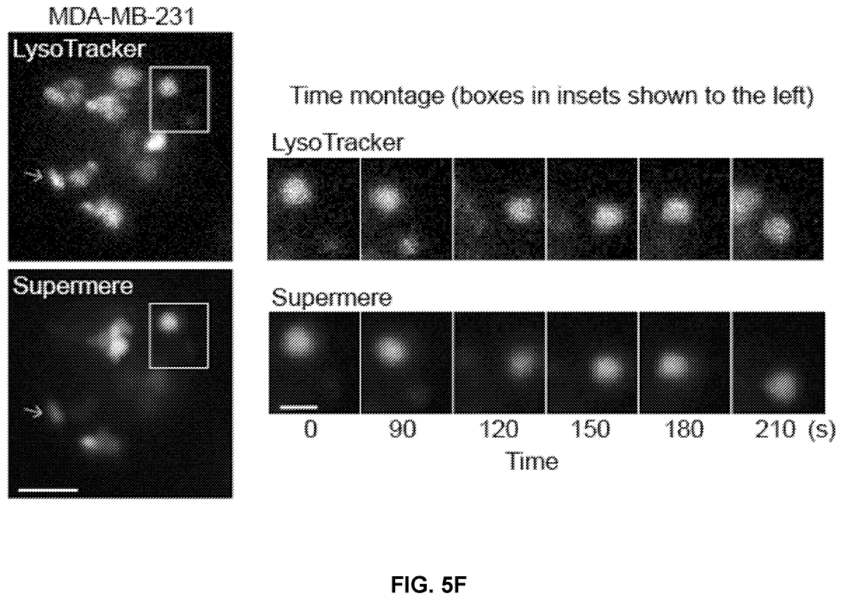

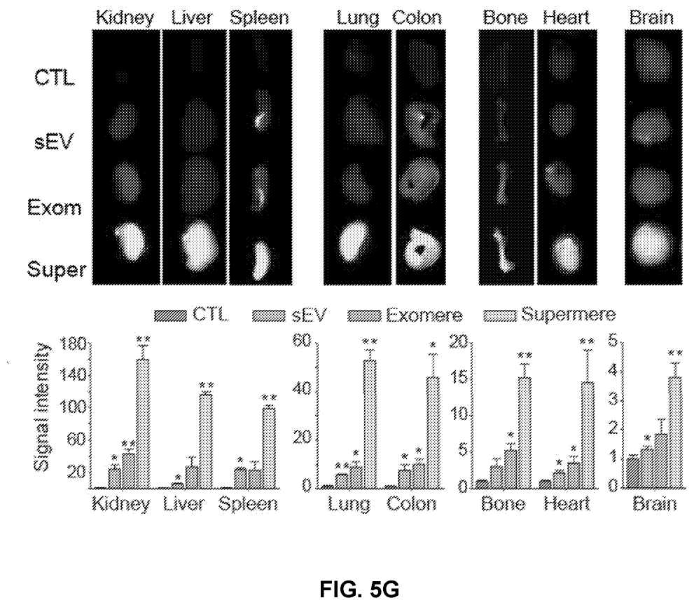

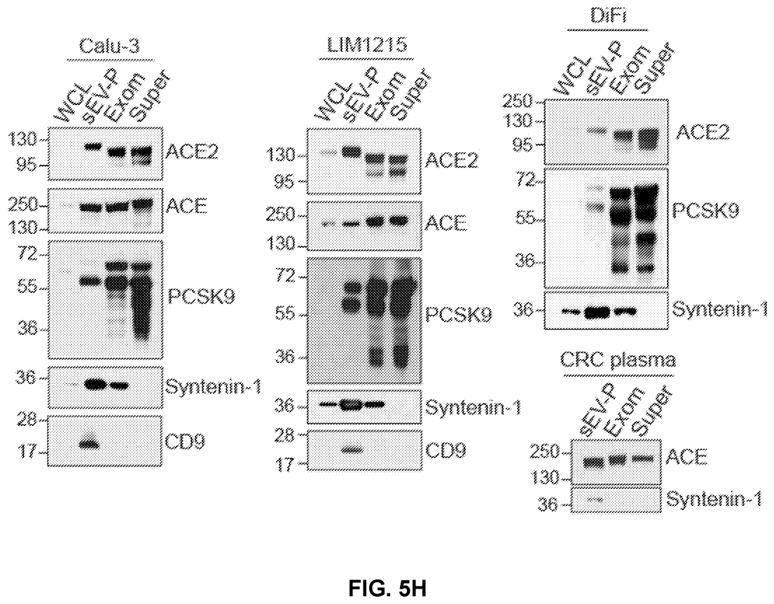

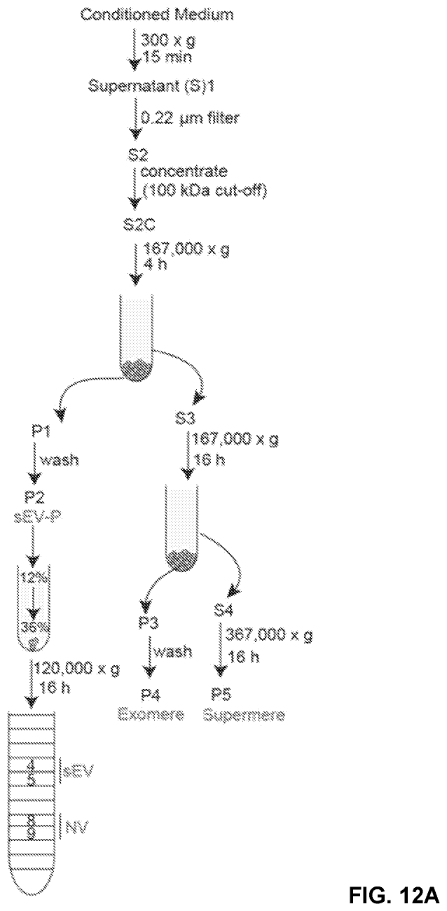

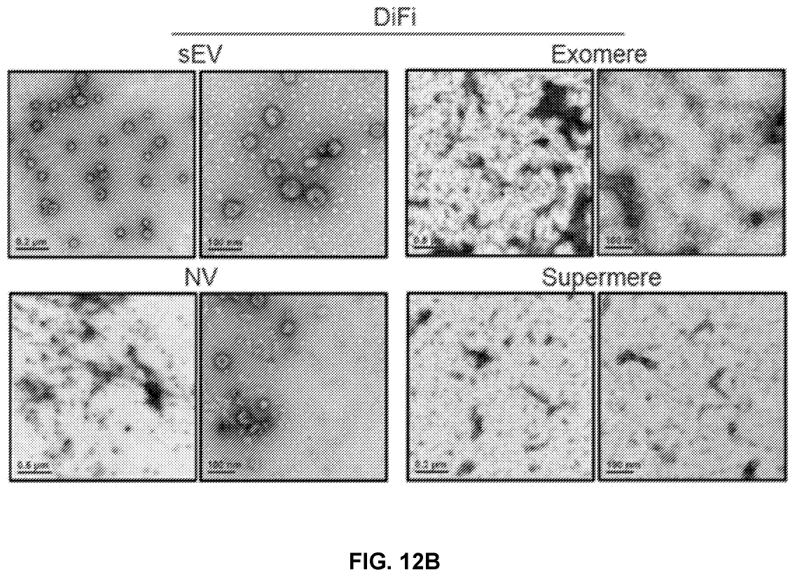

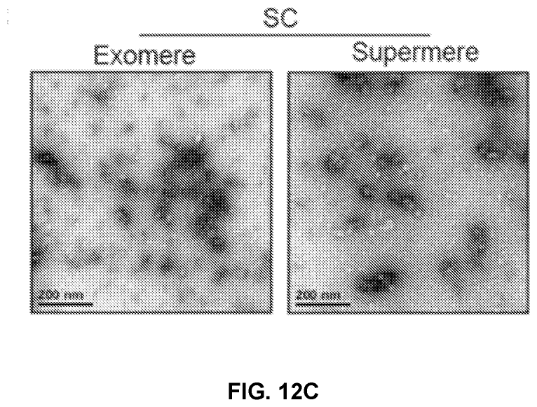

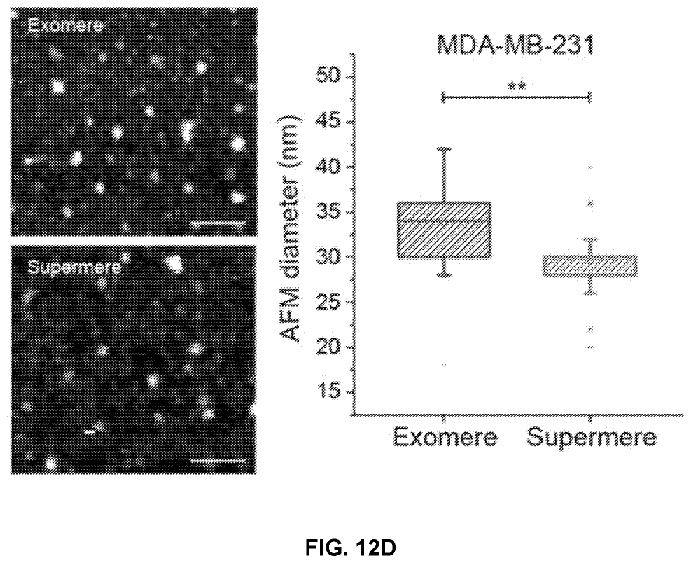

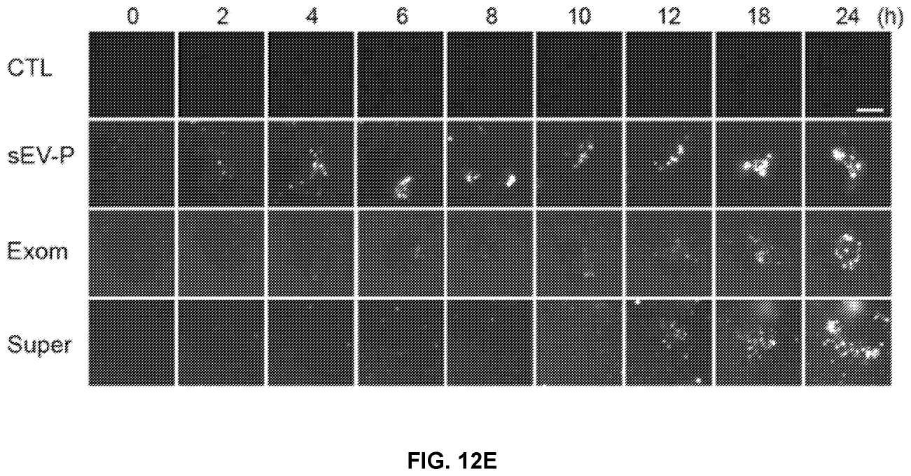

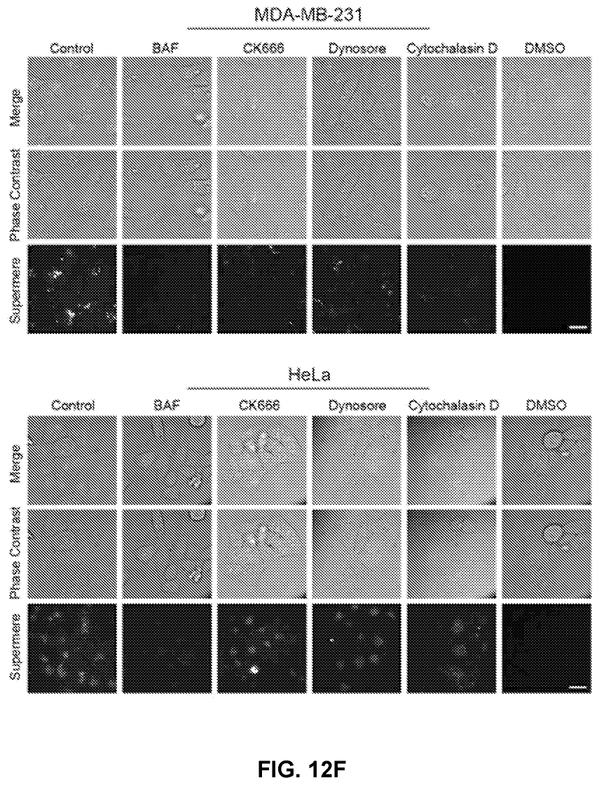

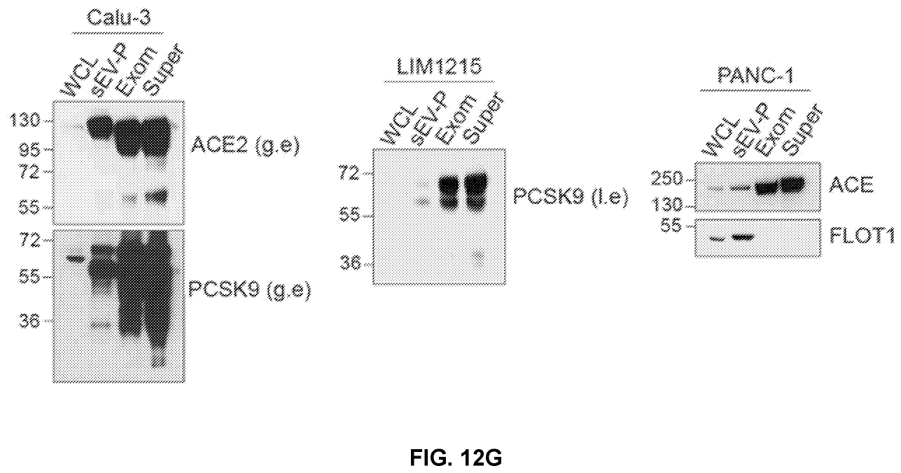

To determine if additional type(s) of nanoparticles could remain after exomere depletion, a sequential high-speed ultracentrifugation protocol (FIG. 5A) was modified. Crude pellets of small extracellular vesicles (sEV-P) were prepared by ultracentrifugation, and for some experiments, the sEV-P samples were further fractionated on high-resolution density gradients to separate highly pure vesicles (sEV) from non-vesicular components (NV), as previously described (Jeppesen, D. K., et al. Cell 2019 177:428-445 e418). Next, exomeres were pelleted, and the resulting supernatants were subjected to ultracentrifugation at 367,000×g (rₘₐₓ, 55,000 RPM) to obtain a pellet of material termed supermeres (FIG. 5A, 12A). Fluid-phase atomic force microscopy (AFM) and transmission electron microscopy (TEM) imaging revealed that the morphological structure of supermeres was distinct from sEVs, NV and exomeres derived from two human CRC cell lines, DiFi and SC (FIG. 5B, 12B, 12C), and from the human breast cancer cell line MDA-MB-231 (FIG. 12D). Under identical force and imaging conditions, supermeres exhibit smaller heights and diameters than other fractions (FIG. 5C,12D).

To investigate uptake dynamics in vitro, sEVs, exomeres and supermeres derived from DiFi cells were fluorescently labeled and then used to treat MDA-MB-231 cells for 24 h. Supermeres and exomeres displayed significantly slower cellular uptake compared to sEVs (FIG. 5D,12E). To examine potential mechanisms of supermere uptake, MDA-MB-231 and Hela cells were pre-treated with inhibitors that block different steps in cellular uptake before adding fluorescently-labeled supermeres. In both MDA-MB-231 and Hela cells, bafilomycin A treatment caused the greatest inhibition of supermere internalization, suggesting that macropinocytosis is likely the main uptake mechanism, although inhibitors targeting endocytosis also significantly reduced internalization (FIG. 5E, 12F). Previous studies have shown that sEVs can enter endo-lysomal compartments, which influence release of vesicle content (Bonsergent, E., et al. Nat Commun 2021 12:1864; Mulcahy, L. A., et al. J Extracell Vesicles 2014 3) or degradation (Choi, D., et al. J Extracell Vesicles 2021 10:e12091). Following internalization, some supermeres also enter endo-lysosomal compartments (FIG. 5F). After completing these in vitro studies, the biodistribution and organ uptake of these different fractions in vivo was next investigated. sEVs, exomeres and supermeres were labeled with a near-infrared dye and injected separately into the peritoneum of C57BL/6 mice. Signal intensity of supermere-treated mice was greatest in the kidney, liver and spleen, indicating that these are the main organs for supermere biodistribution; however, uptake was also high in the lung, colon, bone and heart (FIG. 5G). Consistently, the organ uptake of supermeres was greater than that of either sEVs or exomeres. Strikingly, while there was little uptake of sEVs and exomeres in the brain, as previously reported (Zhang, H., et al. Nat Cell Biol 2018 20:332-343), the uptake of supermeres in the brain was significant (FIG. 5G). Supermeres that cross the blood brain barrier are taken up by oligodendrocytes specifically and may be taken up by other cells as well.

ACE2, the critical entry receptor for the SARS-CoV-2 virus that causes COVID-19, was recently identified in sEVs and exomeres (Zhang, Q., et al. Gastroenterology 2021 160:958-961 e953). Here, it was found that supermeres derived from lung cancer (Calu-3) and CRC (LIM1215 and DiFi) cell lines contain similar levels of ACE2 as exomeres (FIG. 5H,12G). ACE2 is also a biomarker for cardiovascular disease (Narula, S., et al. Lancet 2020 396:968-976) and this led consideration of other circulating cardiovascular biomarkers that might be present in supermeres. The peptidase ACE is a central component of the renin-angiotensin system that controls blood pressure but also functions in innate and adaptive immunity (Bernstein, K. E., et al. Nat Rev Nephrol 2018 14:325-336), while PCSK9 is a circulating serine protease that degrades low-density lipoprotein (LDL) receptors on the surface of cells, thereby playing a pivotal role in regulating the circulating levels of LDL (Stoekenbroek, R. M., et al. Nat Rev Endocrinol 2018 15:52-62). Both ACE and PCSK9 were detected in supermeres derived from these cancer cell lines, and circulating ACE was confirmed in supermeres isolated from human plasma (FIG. 5H,12G). Together, these findings demonstrate that supermeres are secreted nanoparticles with a morphology distinct from that of both exomeres and sEVs, that supermeres circulate in vivo and are efficiently taken up in multiple organs, including the heart and brain, and that they contain cargo relevant to cardiovascular disease.

### Supermeres Exhibit Distinct Proteomic Profiles and the Supermere Protein TGFBI is a Potential CRC Biomarker

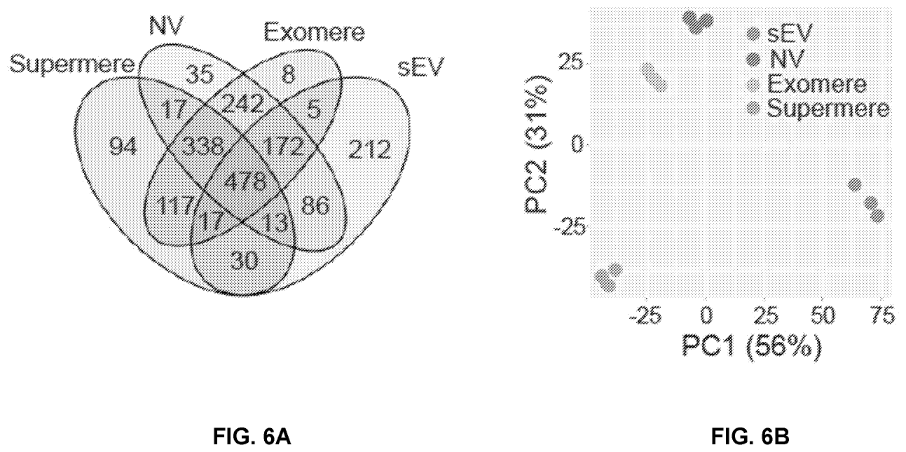

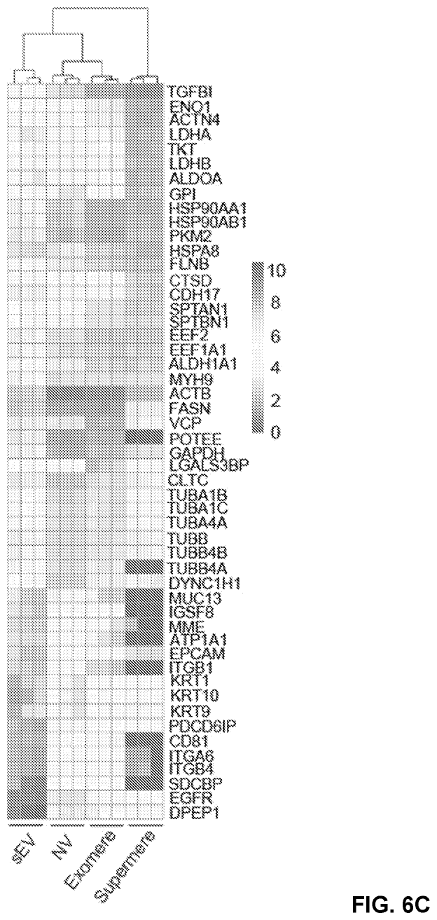

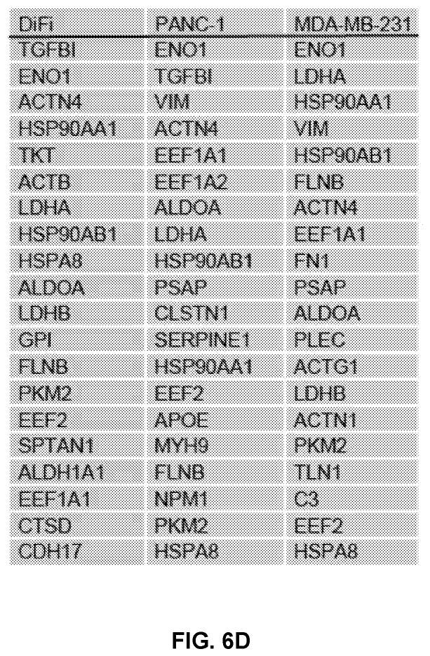

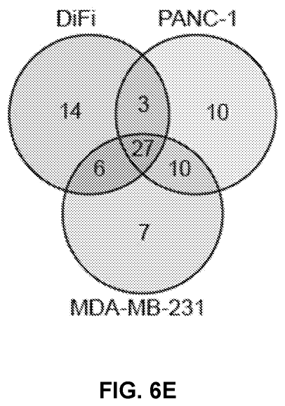

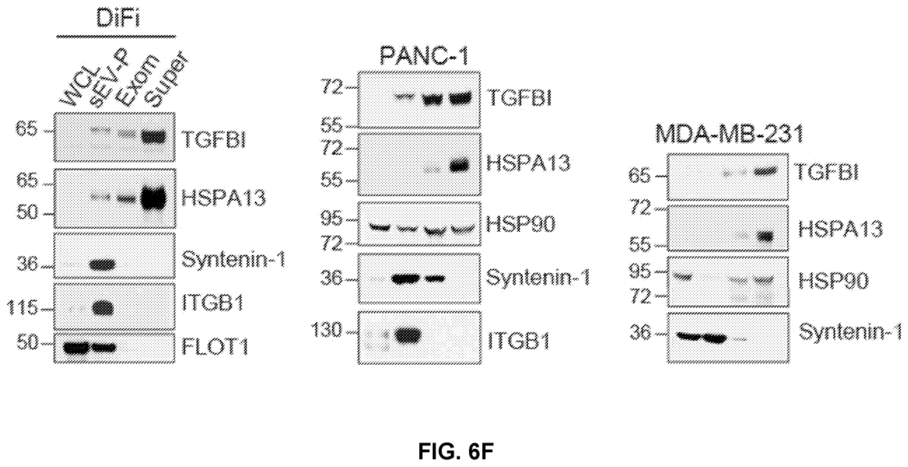

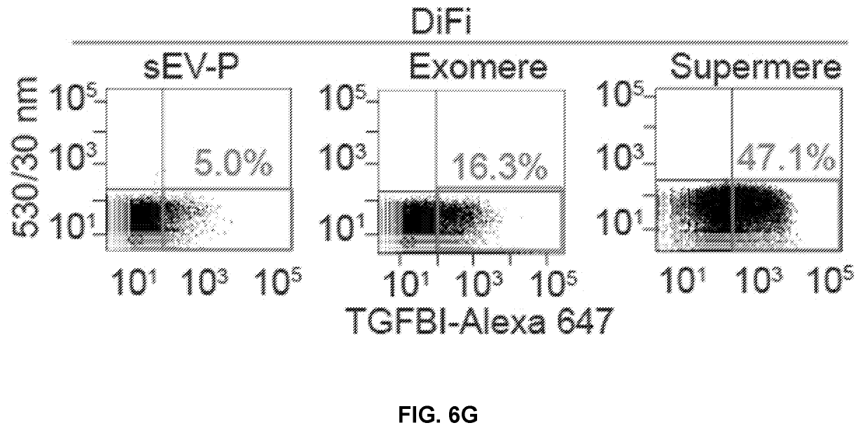

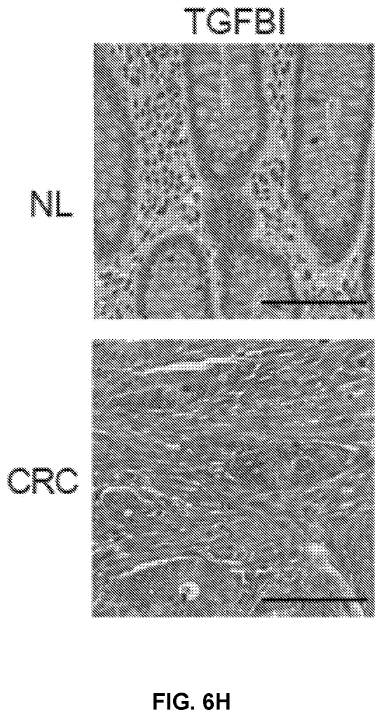

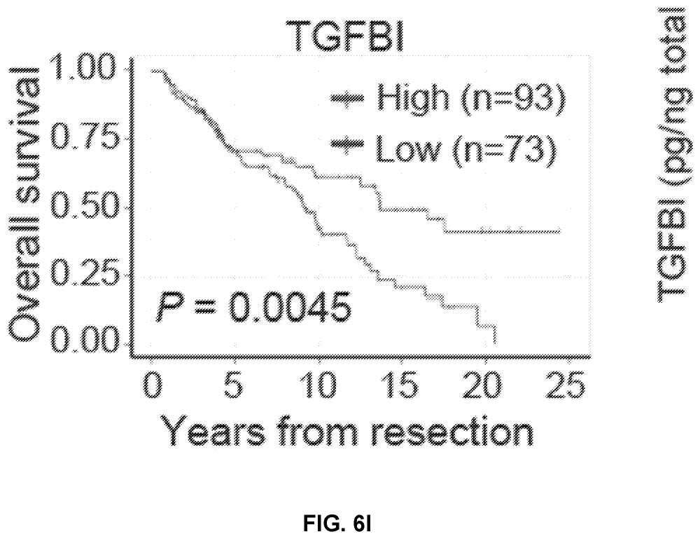

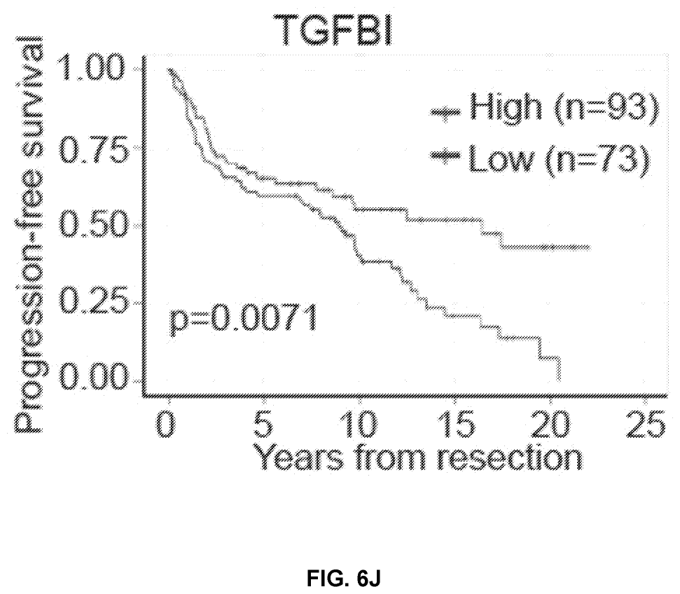

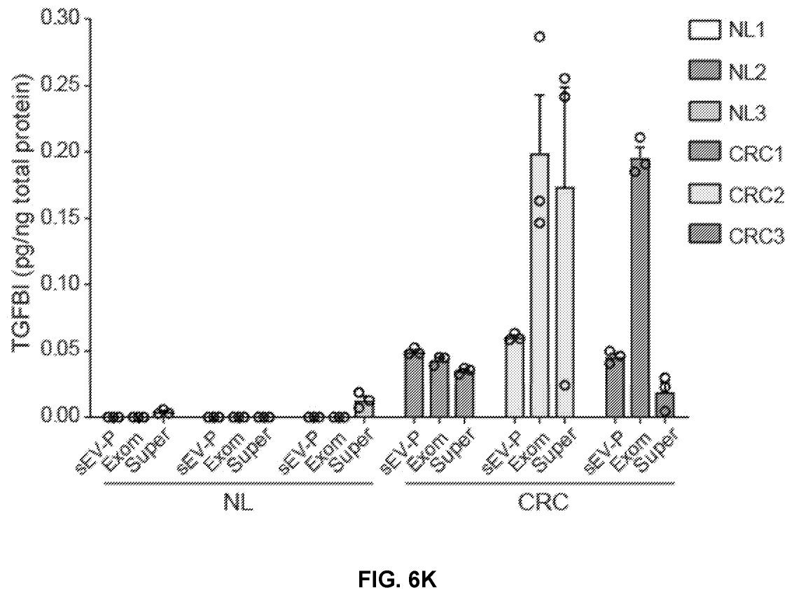

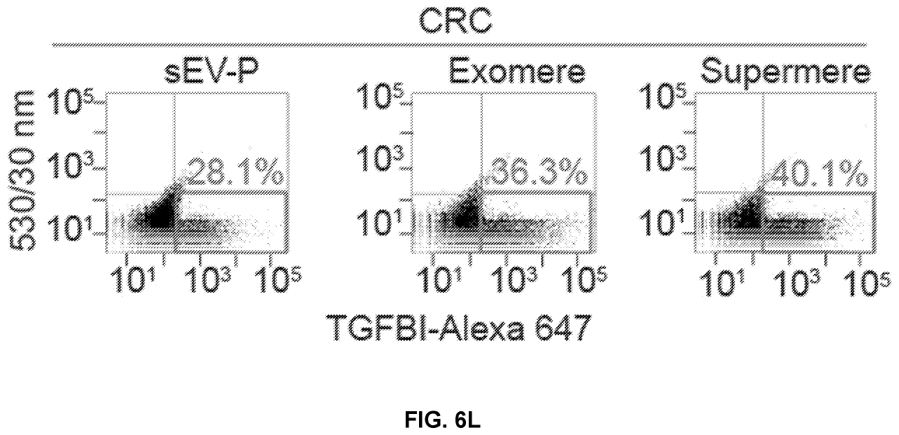

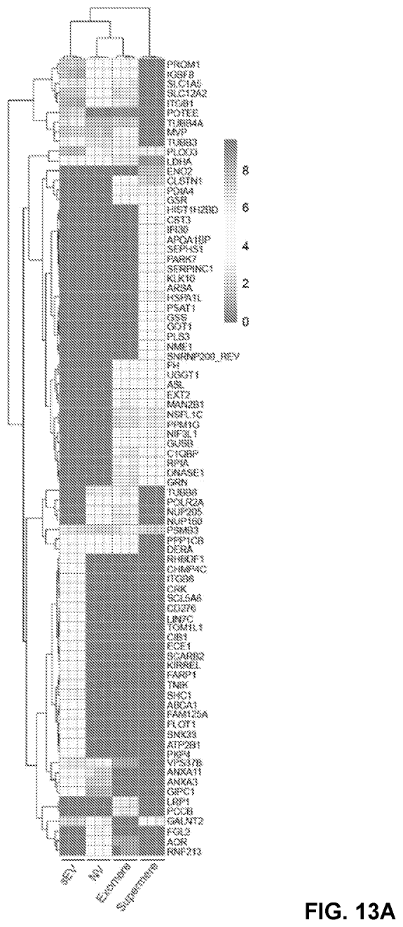

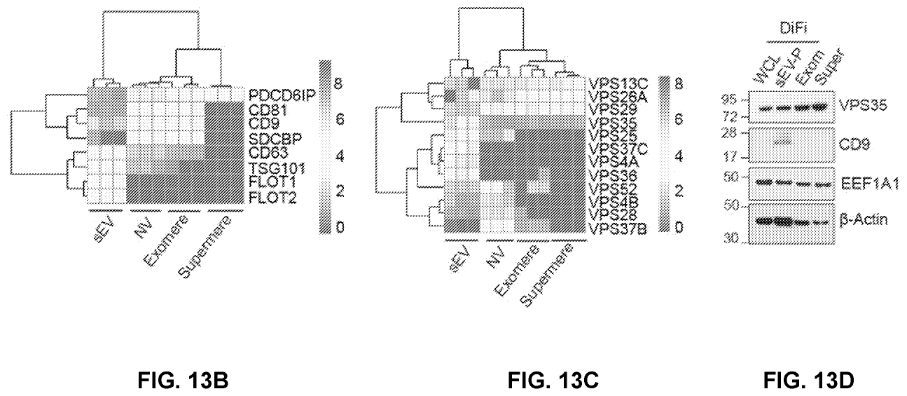

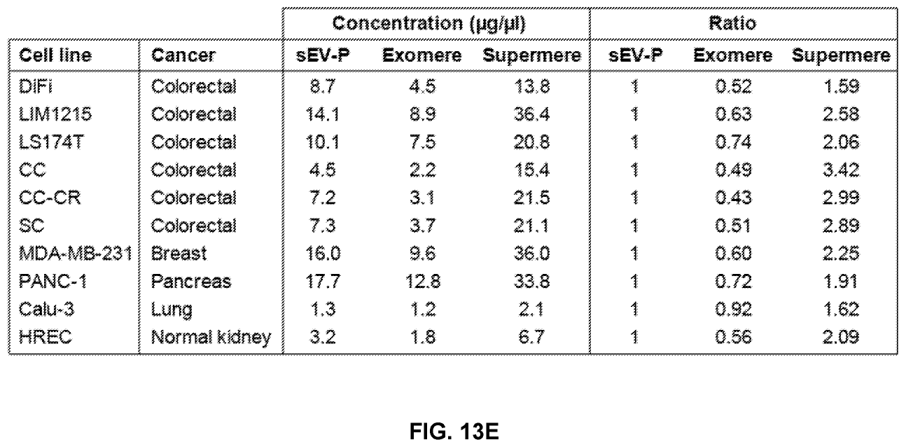

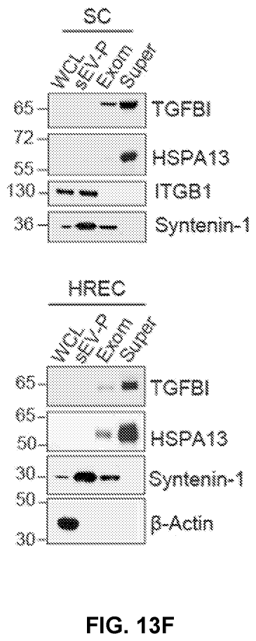

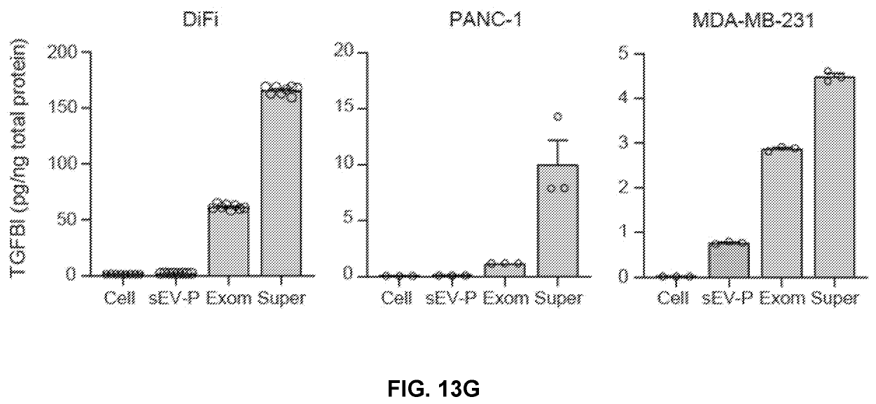

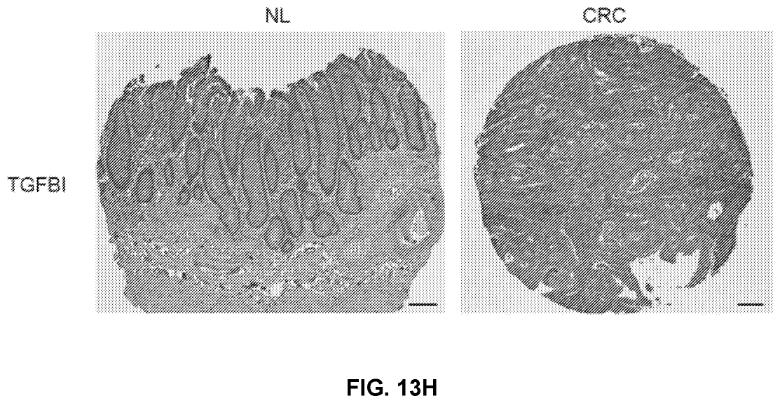

LC/MS-MS proteomics was next performed on gradient-purified sEVs, NVs, exomeres and supermeres. The proteomic profile of supermeres is clearly distinct from that of sEVs, NV and exomeres, while NV and exomeres are more similar (FIG. 6A,6B). Examination of the top 25 most differentially expressed proteins (FIG. 13A) revealed that supermeres were highly enriched in proteins involved in metabolism, including LDHA and ENO2, whereas classical exosomal markers were enriched in sEVs (FIG. 13A, 13B). NV, exomeres and supermeres have a marked enrichment of retromer complex components, VPS35, VPS29 and VPS26A, which mediate retrograde transport of cargo proteins (FIG. 13C,13D). Consistently, across different cancer cell types and primary cultures of human renal epithelial cells (HREC), the yield of supermeres was greater than sEVs while the exomere yield was the lowest (FIG. 13E). TGFBI was the most abundant protein identified in DiFi supermeres and the second most abundant protein in PANC-1 supermeres while the glycolytic enzyme ENO1 was the most abundant protein in PANC-1 and MDA-MB-231 supermeres (FIG. 6C,6D). The presence of TGFBI in supermeres was confirmed by immunoblot (FIG. 6F, 13F), ELISA (FIG. 13G) and FAVS analysis (FIG. 6G). The heat shock protein HSPA13 was highly enriched in supermeres from DiFi, PANC-1, MDA-MB-231, SC and HREC cells, suggesting that HSPA13 may be a useful marker protein for supermeres (FIG. 6F,13F). The heat shock protein HSP90 is highly abundant in supermeres but is less specific for supermeres than HSPA13 (FIG. 6C, 6D, 6F). TGFBI immunohistochemical staining was next examined in a clinically well-annotated CRC tissue microarray. Compared to normal colonic tissue, TGFBI immunoreactivity was greatly increased in CRC, predominantly in the stroma (FIG. 6H, 13H). CRC patients whose tumors had high TGFBI staining had a worse overall survival (FIG. 6I) and progression-free survival (FIG. 6J) by Kaplan-Meier survival analysis. The level of TGFBI measured by ELISA in plasma from three CRC patients was higher than that in three normal individuals (FIG. 6K), and FAVS analysis could detect TGFBI in supermeres isolated from the plasma of an individual with CRC (FIG. 6L). In summary, supermeres display distinct proteomic profiles and TGFBI may be a potential biomarker for CRC. TGFBI is a secreted protein that interacts with matrix components. As such, it has the ability to regulate metastasis and immune cell function. (Corona A, et al. Cell Signal. 2021 84:110028). By manipulating the functionality of supermere-associated TGFBI, it is possible to affect the metastatic potential of tumors and impair the immune surveillance of tumors.

### Supermeres are Enriched in Metabolic Enzymes, Increase Lactate Release and Transfer Cetuximab Resistance

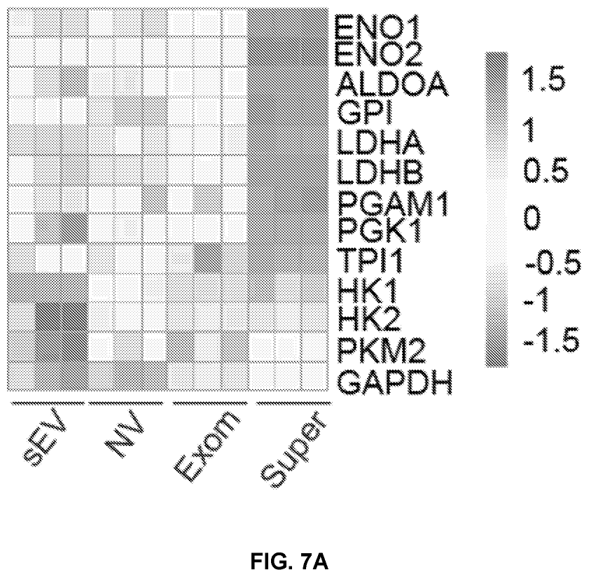

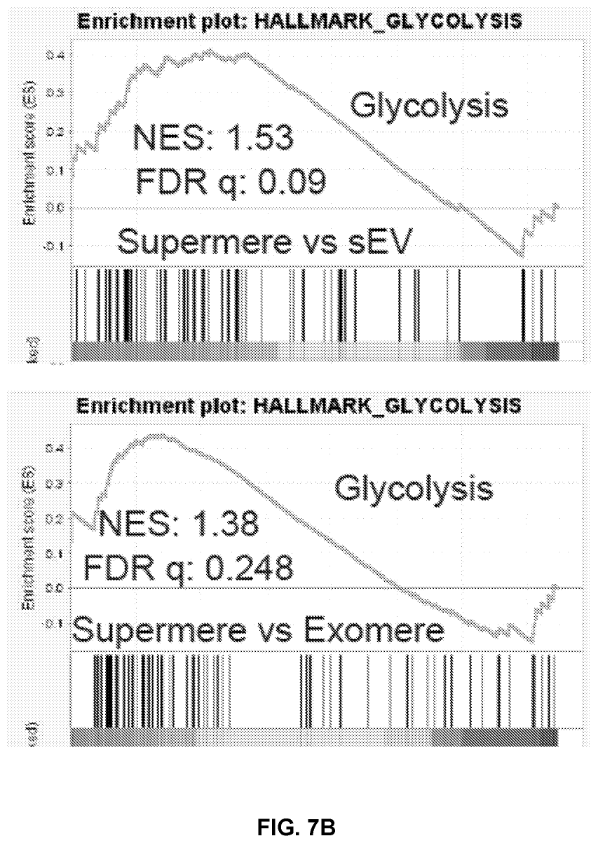

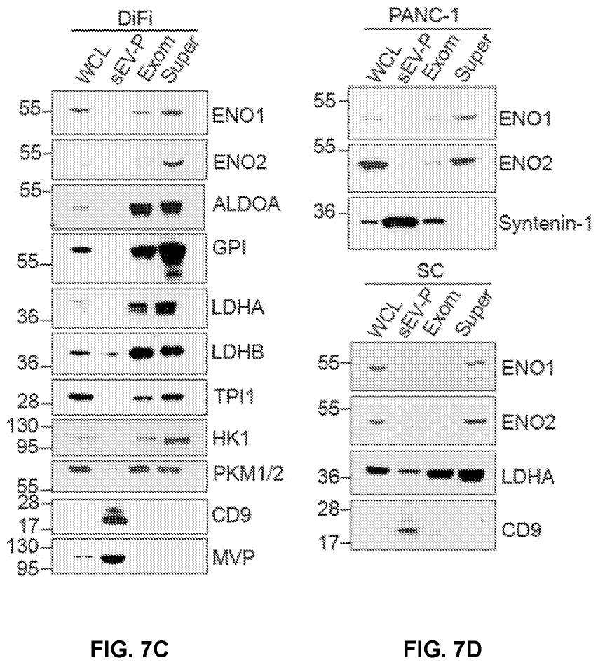

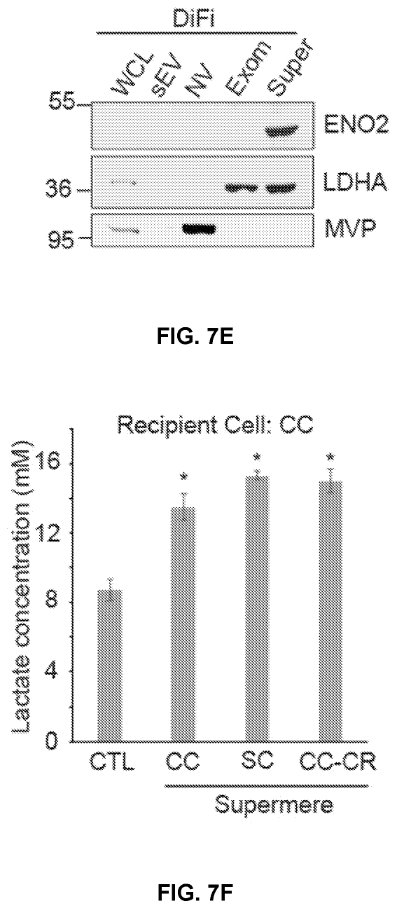

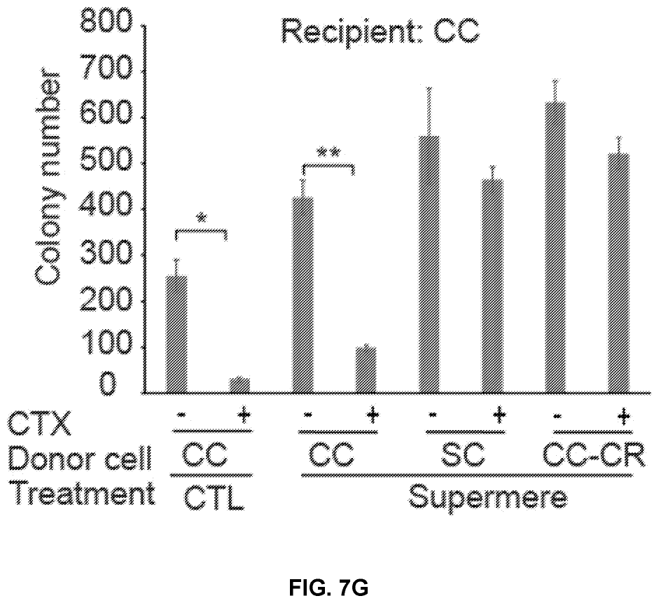

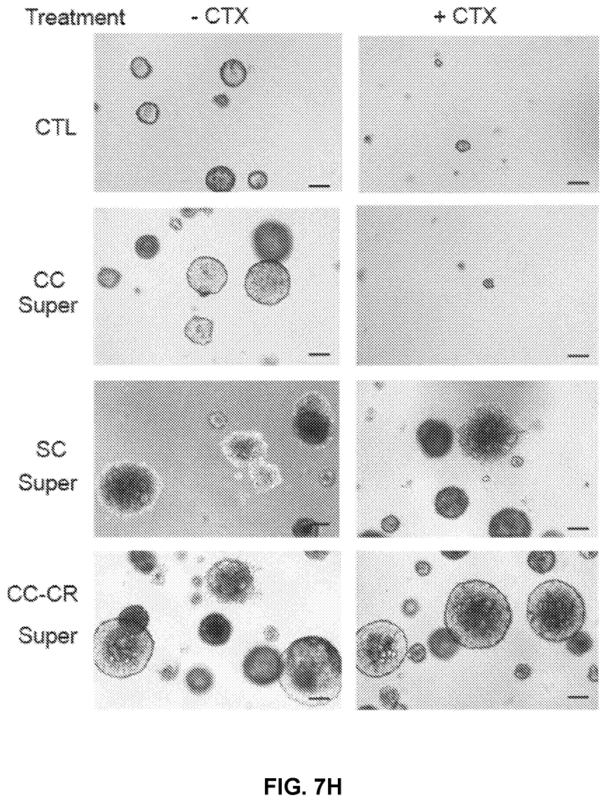

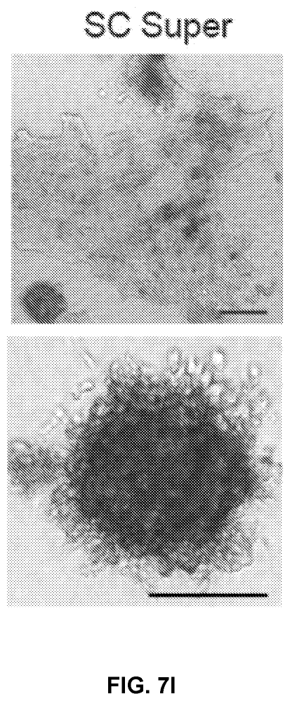

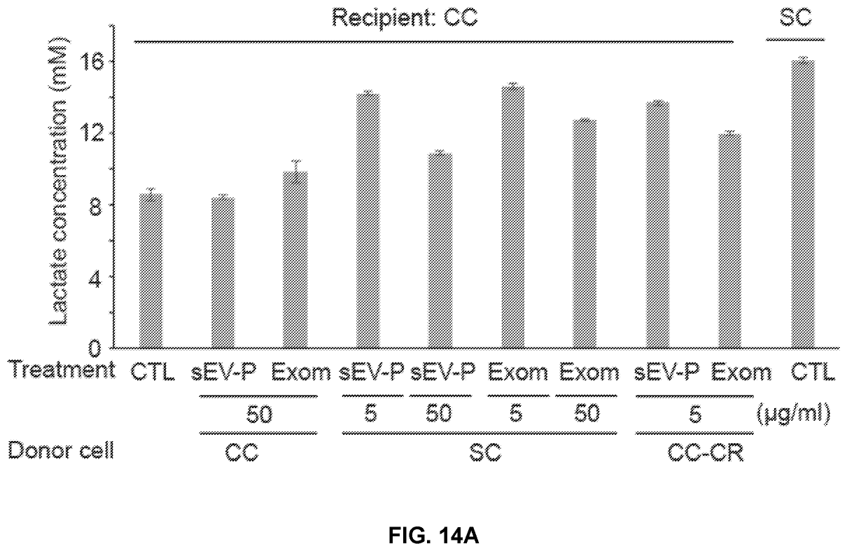

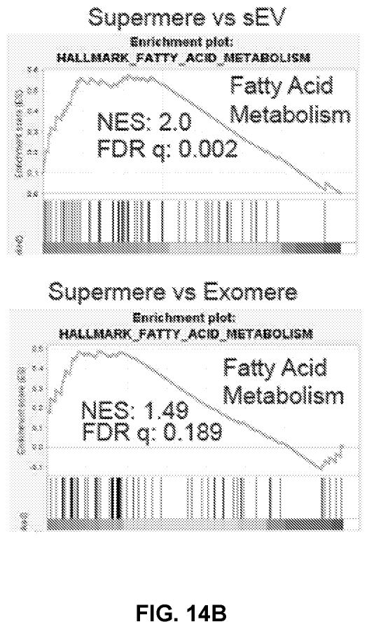

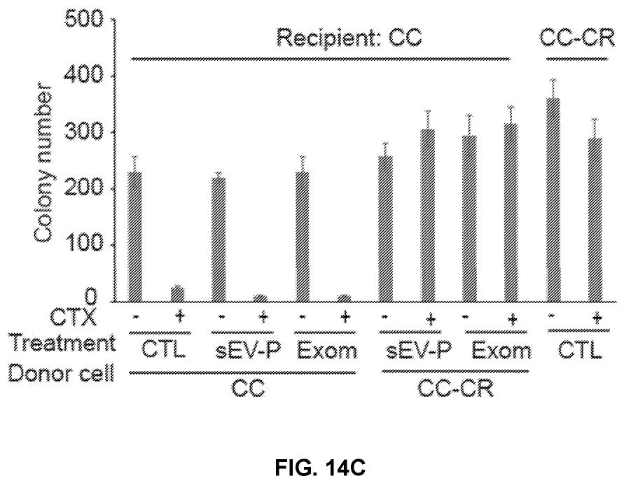

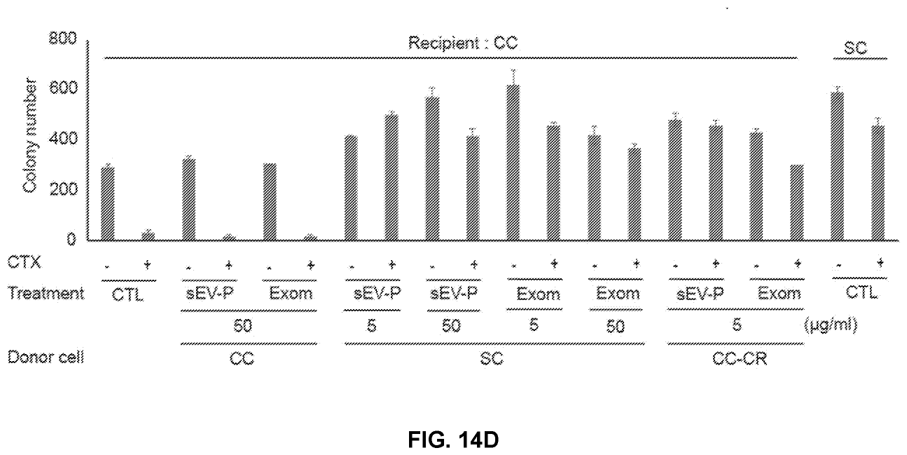

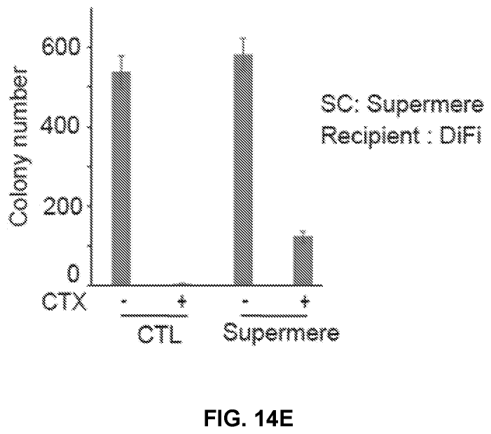

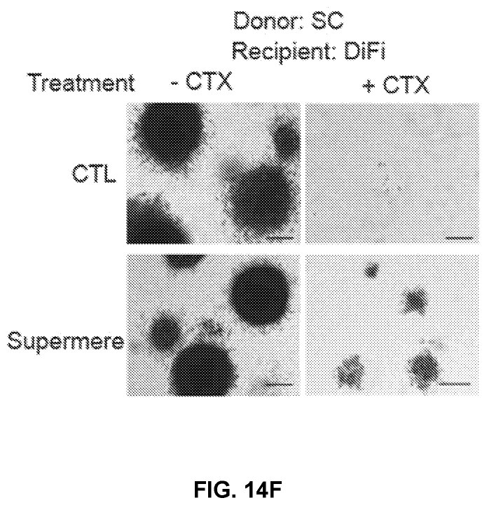

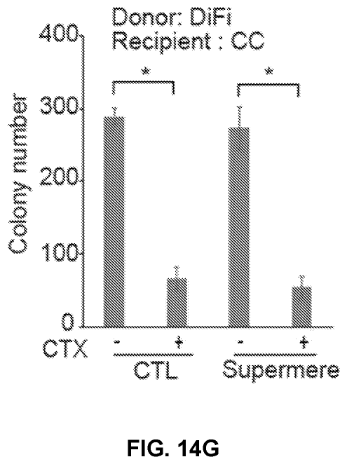

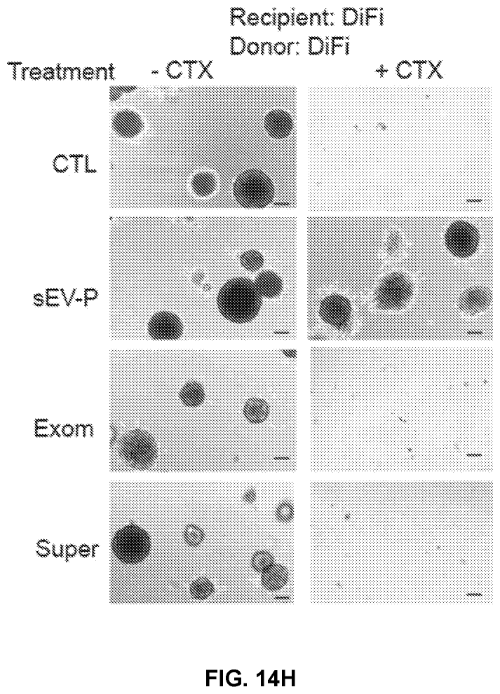

Mutant KRAS exosomes derived from CRC cells can alter the metabolic state of the tumor microenvironment in a non-cell-autonomous fashion that is dependent, at least in part, on the transmembrane glucose transporter GLUT-1 (Zhang, Q., et al. Cell Mol Gastroenterol Hepatol 2018 5:627-629 e626). Since glycolytic enzymes were markedly enriched in supermeres (FIG. 6C,6D), metabolic machinery associated with supermeres was further examined. Enrichment analysis of differentially expressed proteins revealed that many enzymes involved in glycolysis are highly enriched in supermeres compared with sEVs and exomeres (FIG. 7A,7B), as are enzymes involved in fatty acid metabolism (FIG. 14B). The differential expression of glycolytic enzymes was confirmed and ENO2, in particular, was highly associated with supermeres (FIG. 7C-7E). The marked enrichment of glycolytic enzymes prompted us to examine whether supermeres could alter the metabolism of recipient cells by increasing lactate release, a hallmark of the Warburg effect (Brooks, G. A. Cell Metab 2018 27:757-785). Treatment with supermeres derived from CC, cetuximab-resistant CC (CC-CR) (Lu, Y., et al. Nat Med 2017 23:1331-1341), or SC cells greatly increased lactate secretion in CC cells (FIG. 7F), suggesting that release of supermeres may affect the metabolic state. Furthermore, the SC and CC-CR cell-derived sEV-P and exomeres also increased lactate release in CC cells (FIG. 14A), indicating that the release of both EVs and nanoparticles can influence the tumor microenvironment.

Increased lactate secretion has been linked to EGFR and MET drug resistance (Apicella, M., et al. Cell Metab 2018 28:848-865 e846). Initially, the ability of supermeres from cetuximab-resistant cells (SC and CC-CR) to transfer resistance to cetuximab-sensitive cells (CC) cultured in a 3D environment of type-1 collagen was tested (Lu, Y., et al. Nat Med 2017 23:1331-1341; Li, C., et al. Proc Natl Acad Sci USA 2017 114:E2852-E2861). After exposure to CC cell-derived supermeres, CC cells in 3D culture remained sensitive to the growth inhibitory effects of cetuximab (FIG. 7G). In marked contrast, after exposure to SC or CC-CR supermeres, CC cells were no longer growth inhibited by cetuximab (FIG. 7G). Transfer of cetuximab resistance was also observed by treatment of CC cells with the sEV-P and exomeres from SC and CC-CR cells (FIG. 14C, 14D). Supermeres from CC cells stimulated the growth of CC colonies, whereas SC and CC-CR cell-derived supermeres stimulated CC colony growth regardless of the presence of cetuximab. Also, addition of supermeres from SC and CC-CR cells led CC colonies to morphologically resemble that of the donor cell (FIG. 7H). Some of the CC colonies treated with SC cell-derived supermeres displayed a spreading or migratory phenotype, whereas others exhibited multiple protrusions (FIG. 7I). Furthermore, SC supermeres can also transfer cetuximab resistance to highly cetuximab-sensitive DiFi cells, although to a lesser extent compared to CC cells (FIG. 14E,14F). However, DiFi cell-derived supermeres failed to confer cetuximab resistance in CC and DiFi cells, suggesting that transfer of cetuximab resistance by supermeres is dependent on the resistance status of the donor cell (FIG. 7J, 14G, 14H).

In summary, supermeres are functional extracellular nanoparticles. Supermeres are enriched in glycolytic enzymes and can increase lactate release in recipient cells, and supermeres derived from cetuximab-resistant cells are able to transfer resistance to cetuximab-sensitive recipient cells in a 3D cell culture system.

### Supermeres and Exomeres are Highly Enriched in Clinically Relevant Shed Membrane Proteins

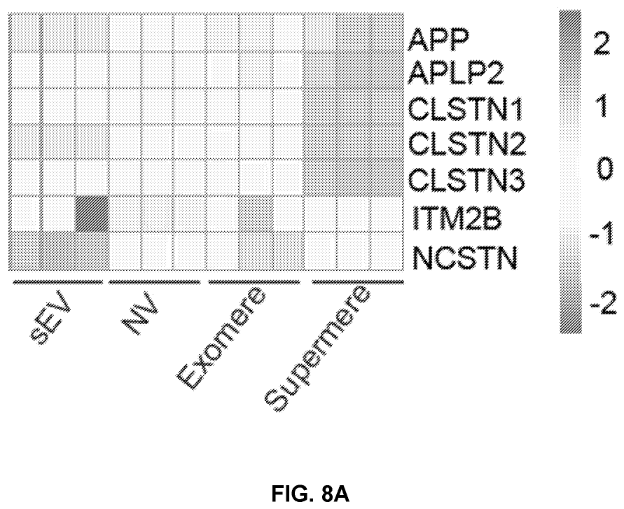

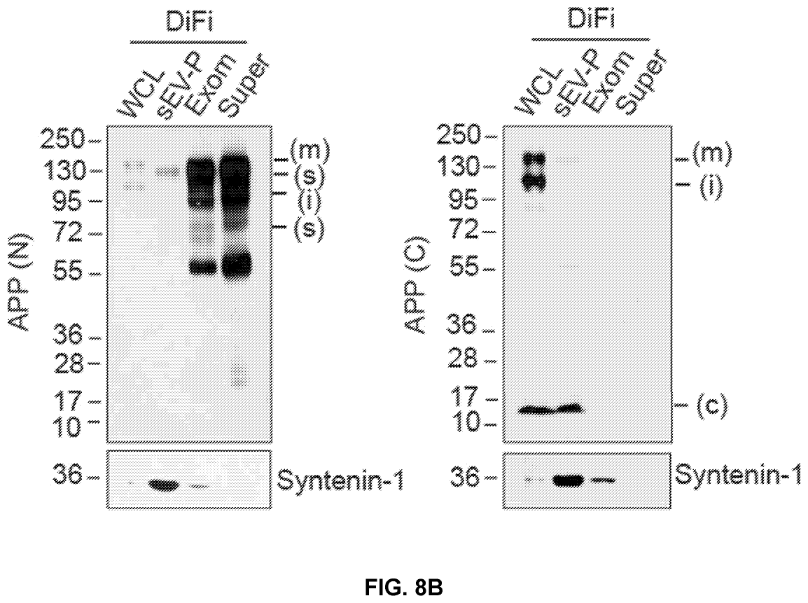

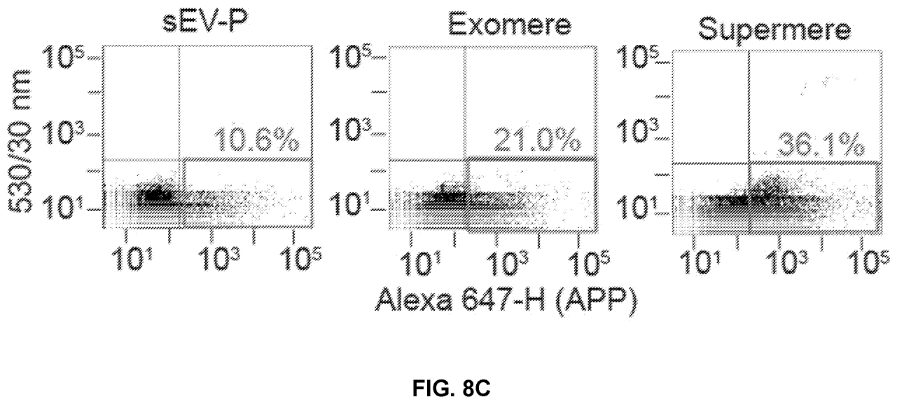

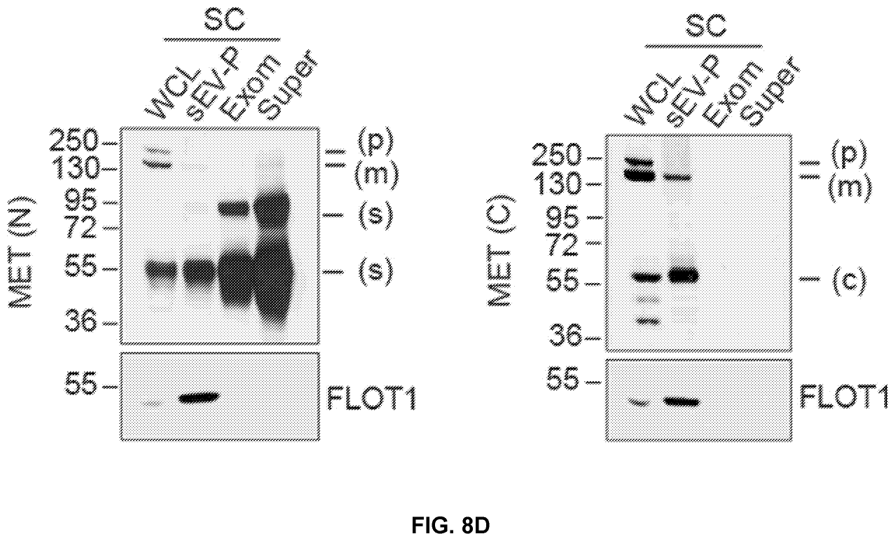

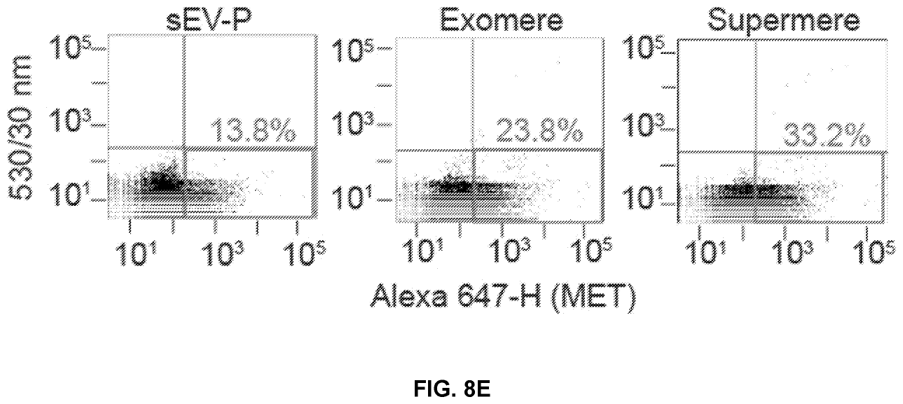

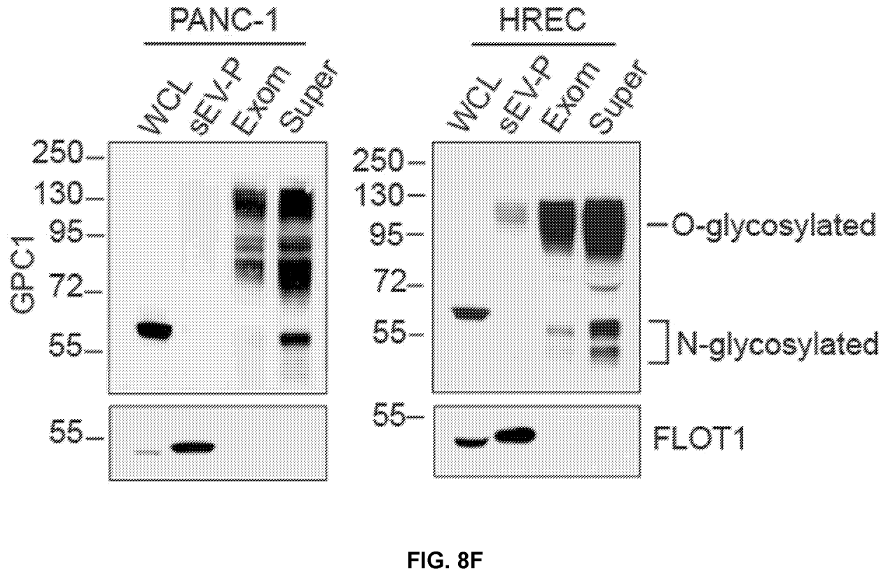

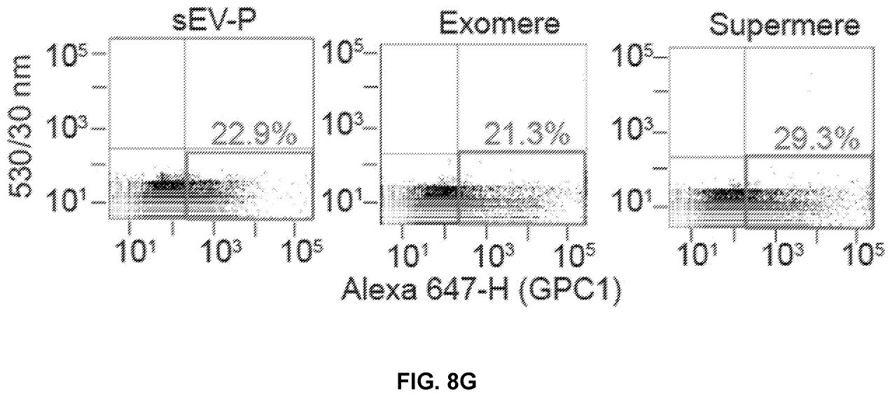

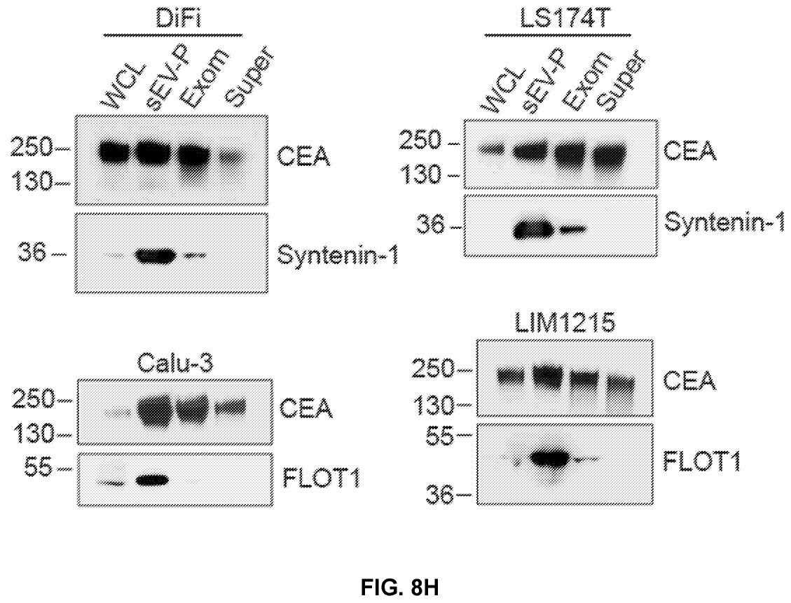

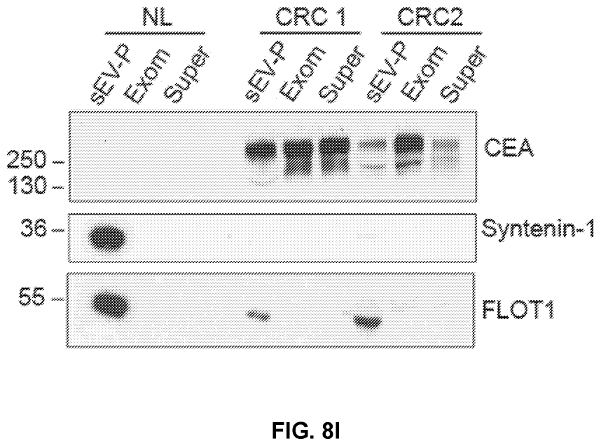

As there was a greater brain uptake of supermeres compared to both sEVs and exomeres (FIG. 5G), it was decided to examine APP as C-terminal fragments of this protein have previously been observed in exosomes (Miranda, A. M., et al. Nat Commun 2018 9:291). APP, a cell surface transmembrane precursor protein, is sequentially cleaved by α-, β- and γ-secretases to generate soluble APPs, C-terminal fragments and amyloid beta, which are essential to the pathogenesis of Alzheimer's disease (Barao, S., et al. Trends Neurosci 2016 39:158-169). APP and other membrane proteins associated with APP or Alzheimer's disease, including APLP2, underwent ectodomain shedding and were highly enriched in supermeres (FIG. 8A). The enrichment of ectodomain APP in exomeres and supermeres derived from both DiFi and SC cells, and the confinement of full-length APP to cells and sEVs, was confirmed by immunoblotting with antibodies specific to ectodomain (N-terminal) or cytoplasmic (C-terminal) epitopes (FIG. 8B, 15A). Enrichment of APP in supermeres was further confirmed by FAVS analysis (FIG. 8C).

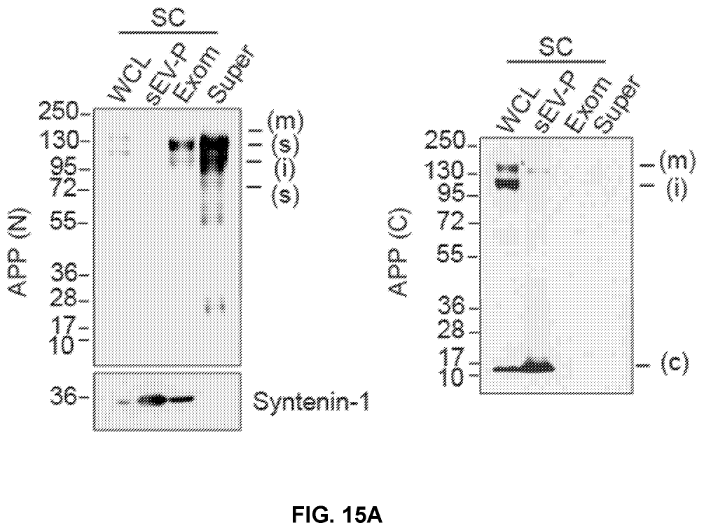

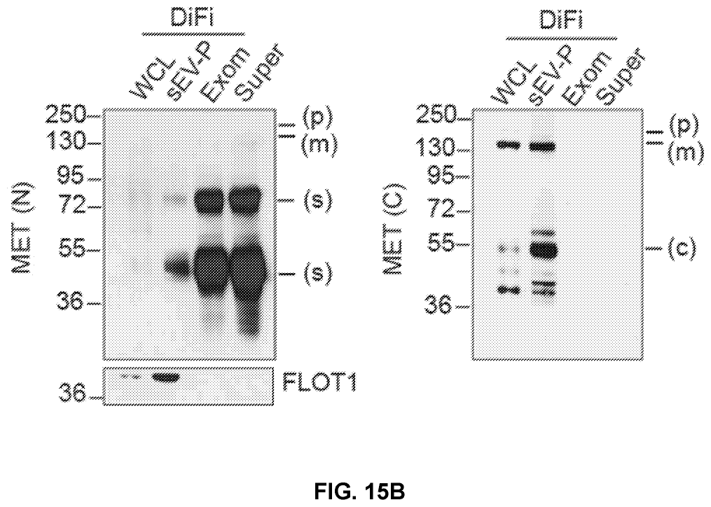

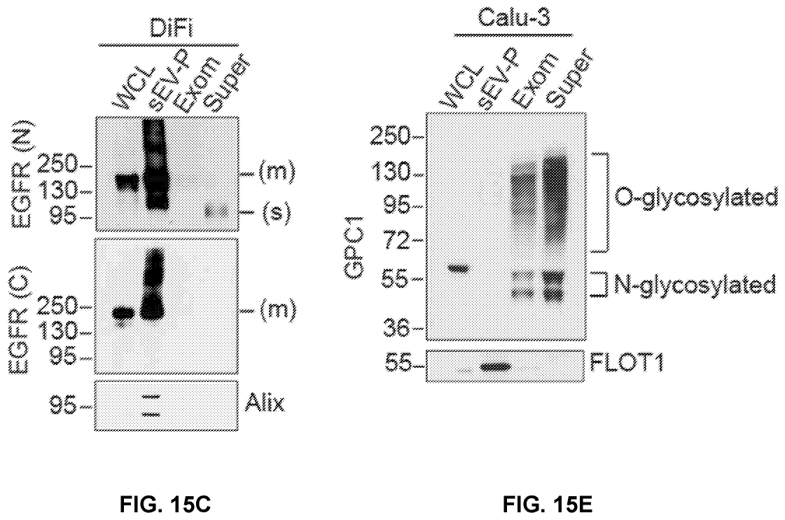

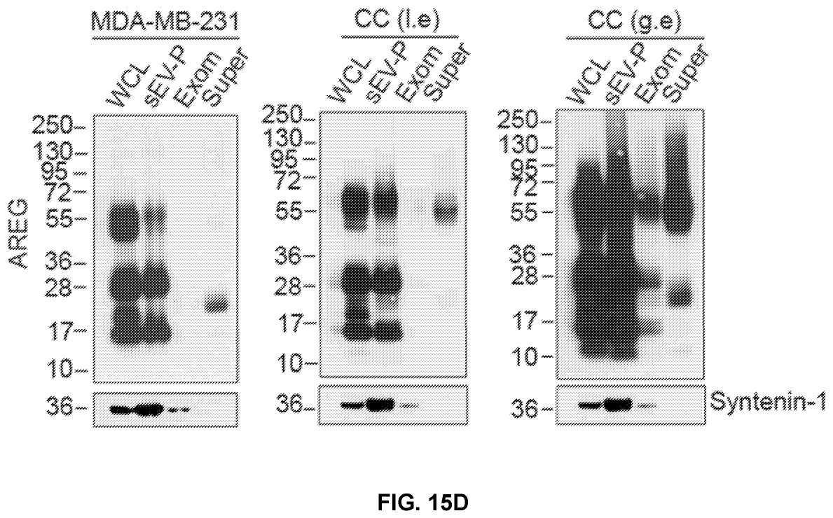

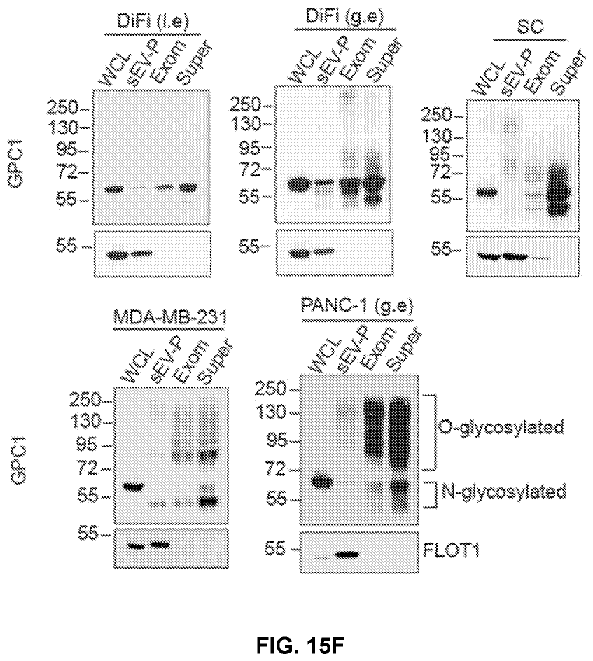

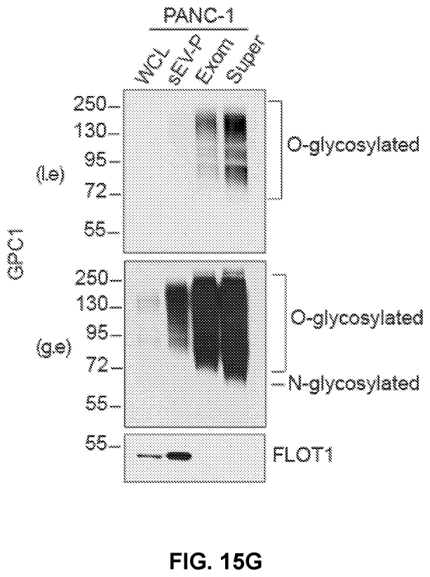

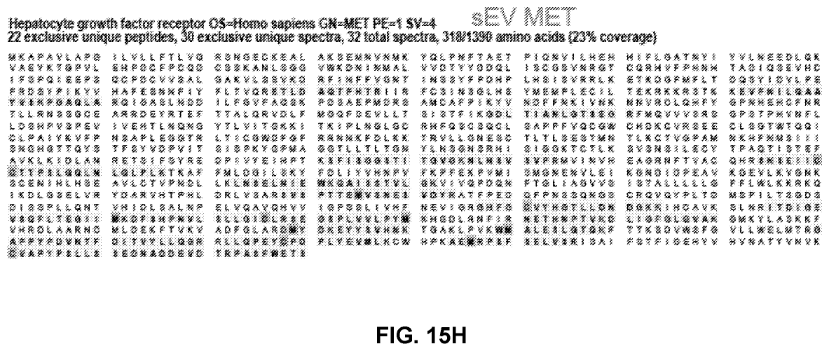

MET, a receptor tyrosine kinase, is deregulated in many types of human cancers and mediates diverse biological responses, including metastasis (Comoglio, P. M., et al. Nat Rev Cancer 2018 18:341-358); MET transfer by exosomes has been proposed to increase the metastatic behavior of primary tumors (Peinado, H., et al. Nat Med 2012 18:883-891). Proteomics data indicated that full-length MET was present in sEVs, while only peptides covering the ectodomain were present for supermeres and exomeres (FIG. 15H). Immunoblotting with antibodies specific to ectodomain (N-terminal) or cytoplasmic (C-terminal) epitopes of MET revealed that shed ectodomain MET was highly enriched in supermeres released from SC (FIG. 8D) and DiFi cells (FIG. 15B), while the membrane-embedded, full-length MET was only detected in cells and sEVs. These results were confirmed with FAVS analysis (FIG. 8E). Shed ectodomains of EGFR were observed in supermeres released from DiFi cells (FIG. 15C), and the ectodomain of the EGFR ligand amphiregulin (AREG) was observed in supermeres from MDA-MB-231 and CC cells (FIG. 14D).

GPC1 is a GPI-anchored cell surface heparan sulfate proteoglycan that is overexpressed in several cancers, including pancreatic cancer and CRC (Xu, R., et al. Nat Rev Clin Oncol 2018 15:617-638). GPC1 in plasma exosomes is reportedly a useful biomarker for the early detection of pancreatic cancer (Melo, S. A., et al. Nature 2015 523:177-182). However, different forms of GPC1 were far more associated with the non-vesicular exomere and supermere nanoparticles released from pancreatic cancer PANC1 cells and normal human renal epithelial cells (HREC) (FIG. 8F, 15F, 15G). Similar results were observed for the Calu-3, DiFi, SC and MDA-MB-231 cell lines (FIG. 15E, 15F). Once again, validation was obtained by FAVS analysis (FIG. 8G). CEA, another GPI-anchored protein, is clinically used as a biomarker to monitor tumor recurrence in CRC patients (Duffy, M. J., et al. Int J Cancer 2014 134:2513-2522). CEA was present in sEVs, exomeres and supermeres derived from DiFi, LS174T, LIM1215 and Calu-3 cells (FIG. 8H). Furthermore, CEA was highly enriched in plasma sEVs, exomeres and supermeres from CRC patients but was not detected in plasma from normal individuals (FIG. 8I).

In summary, exomere and supermere nanoparticles are enriched in many shed, clinically relevant, membrane proteins, including APP, MET, GPC1, CEA, EGFR, AREG, ACE and ACE2, and can be detected by optimized flow cytometry.

Differential Expression of Small exRNAs Among sEVs, Exomeres and Supermeres

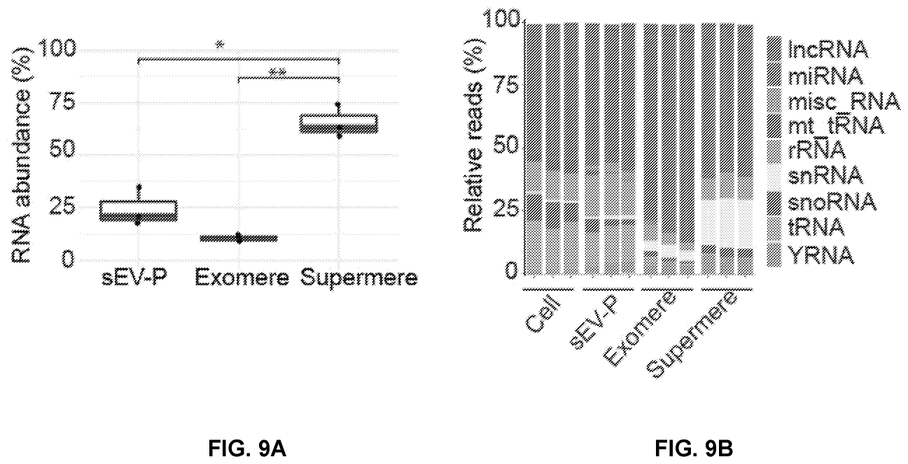

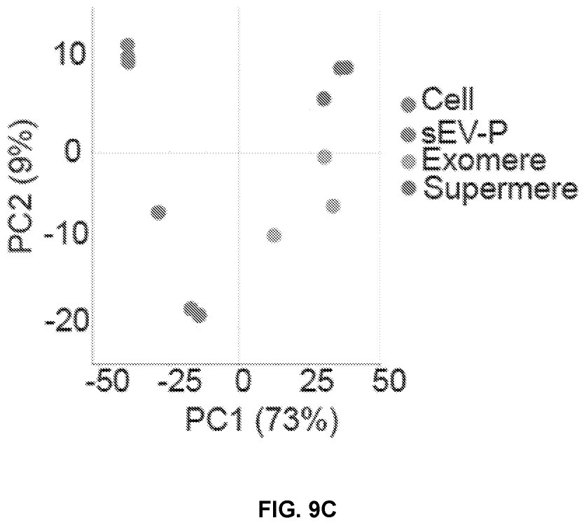

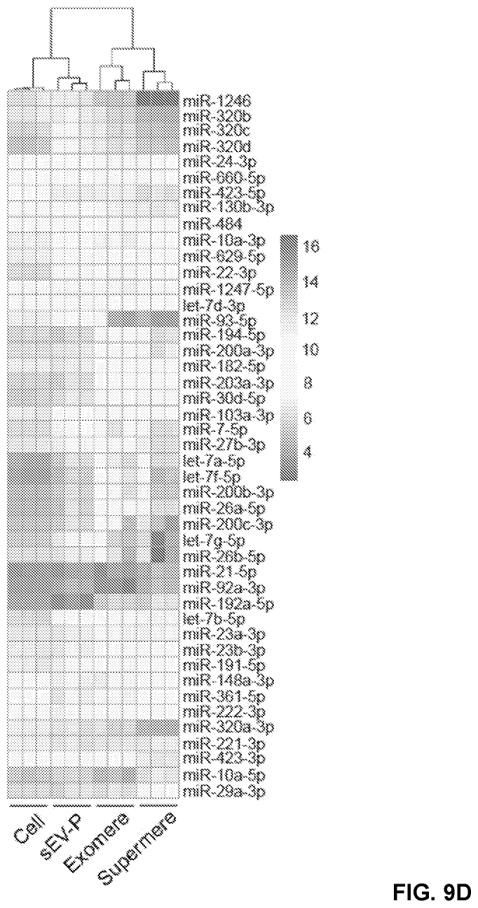

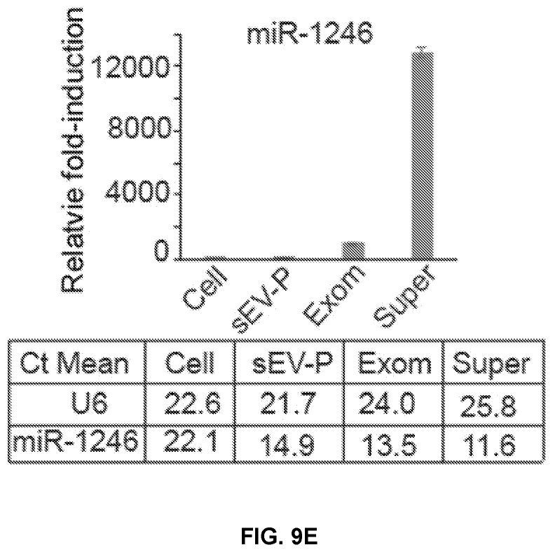

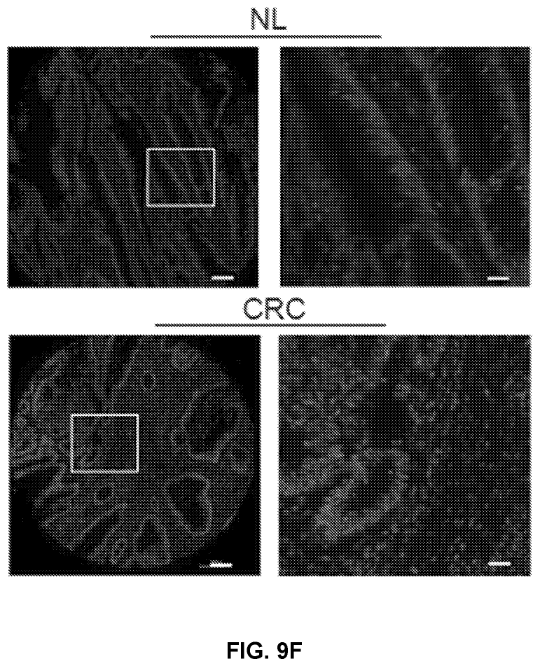

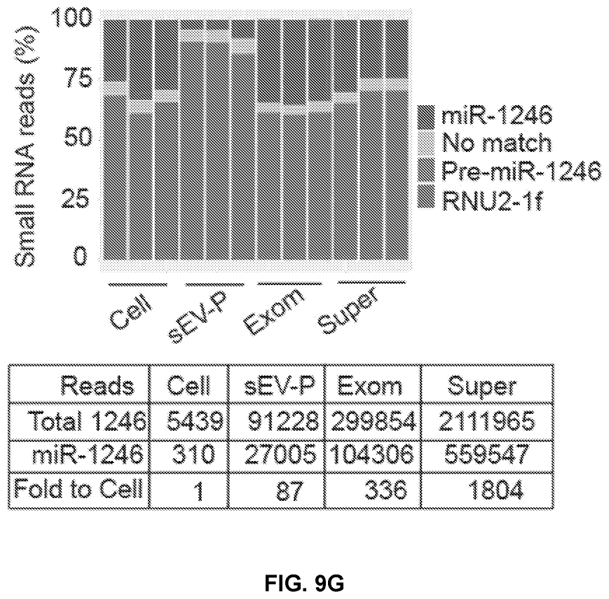

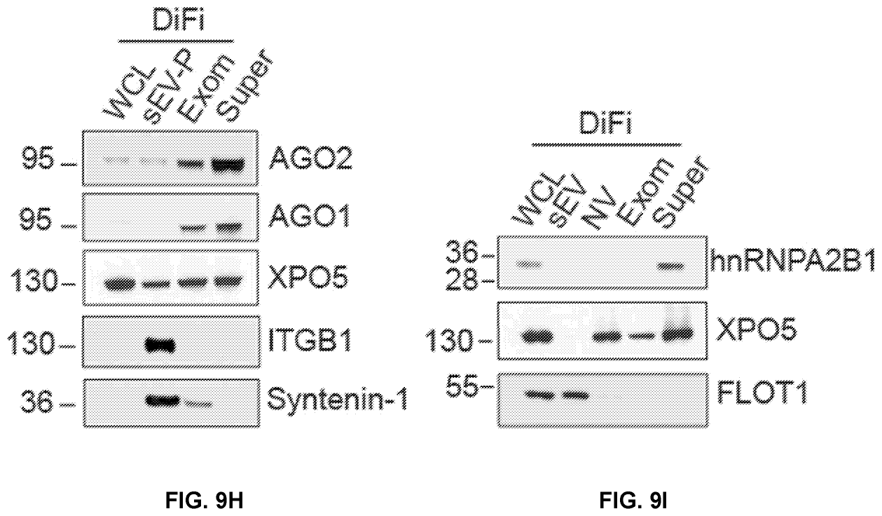

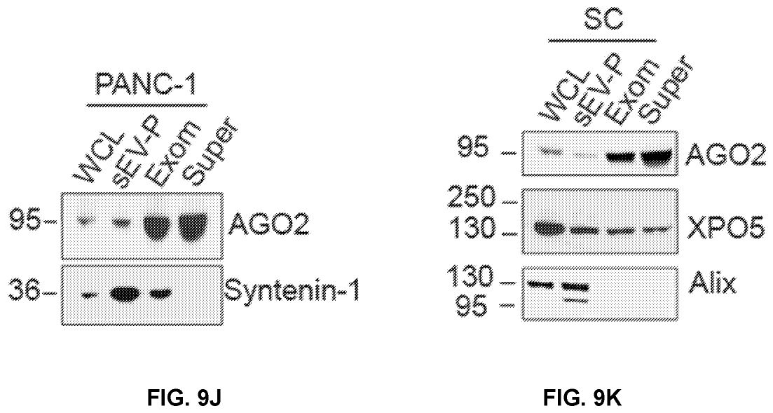

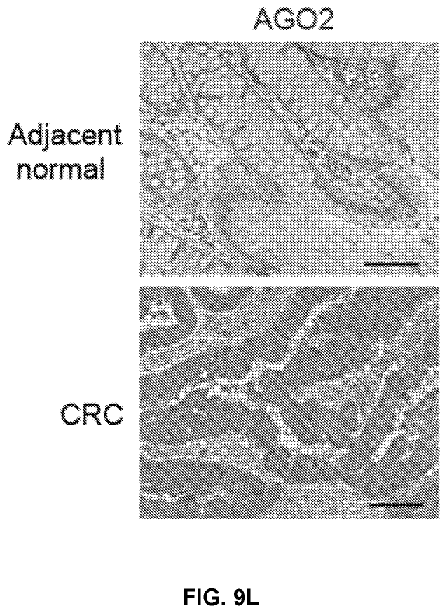

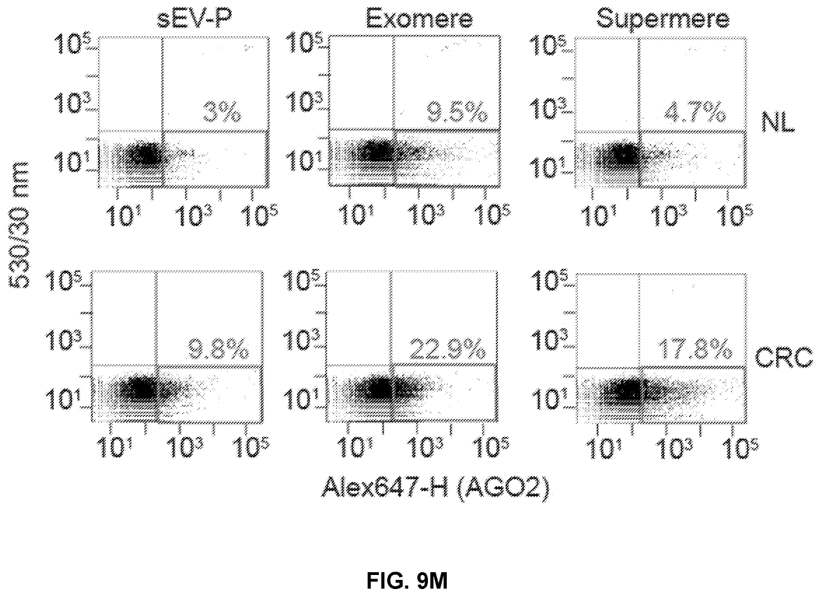

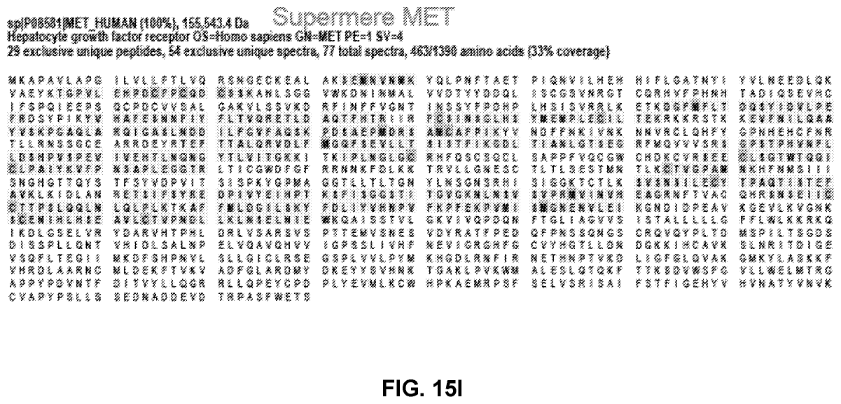

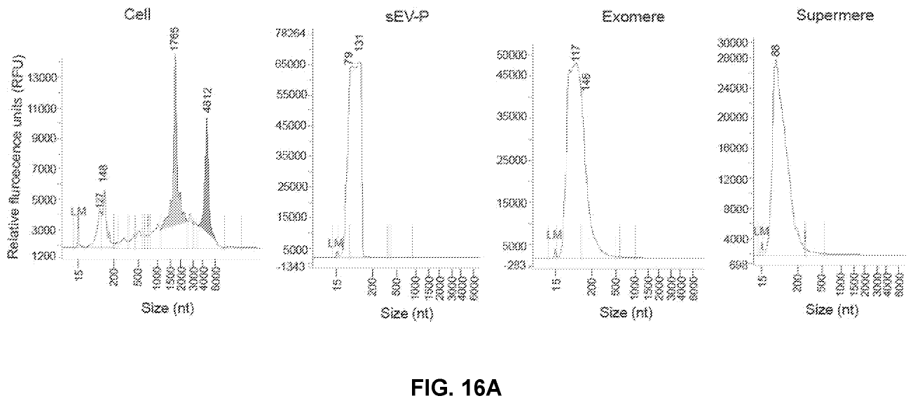

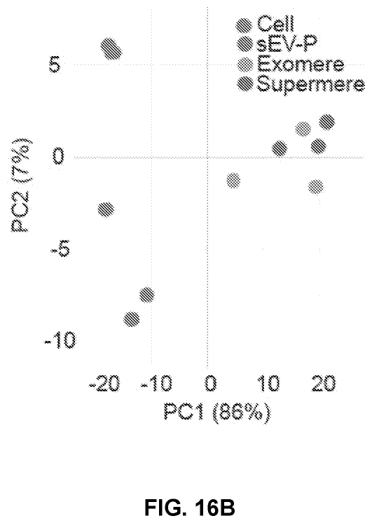

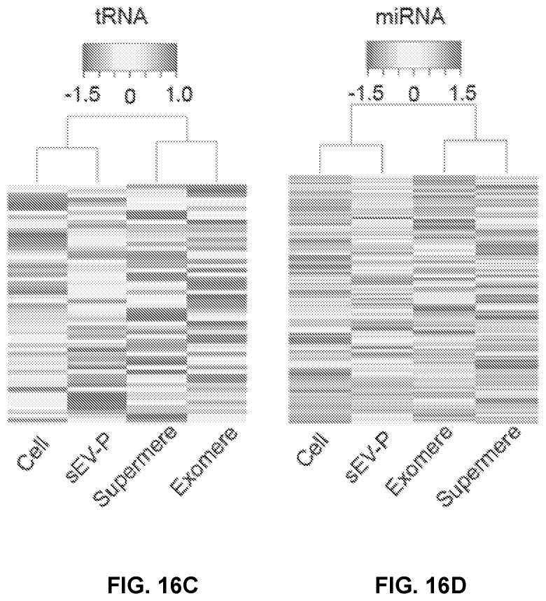

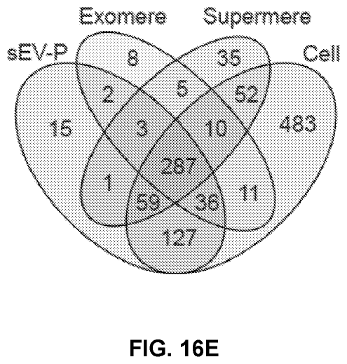

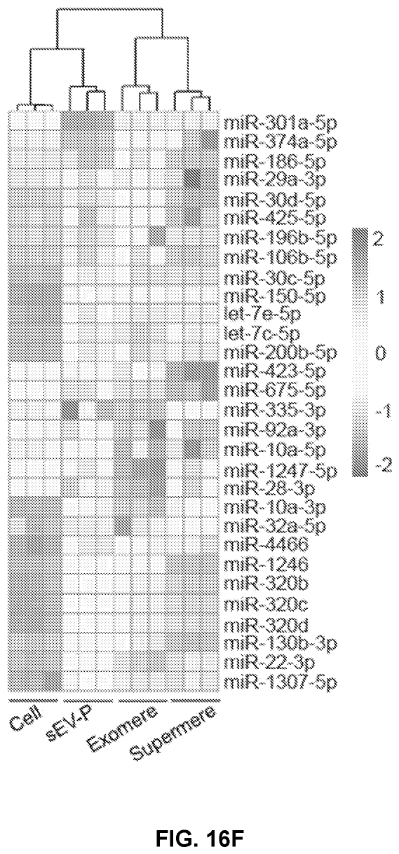

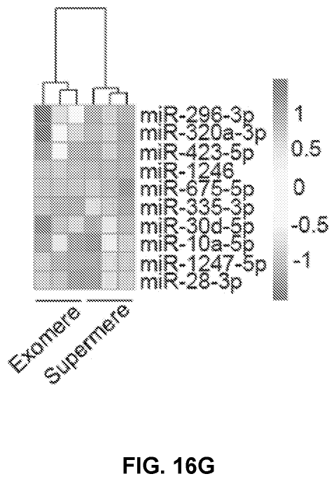

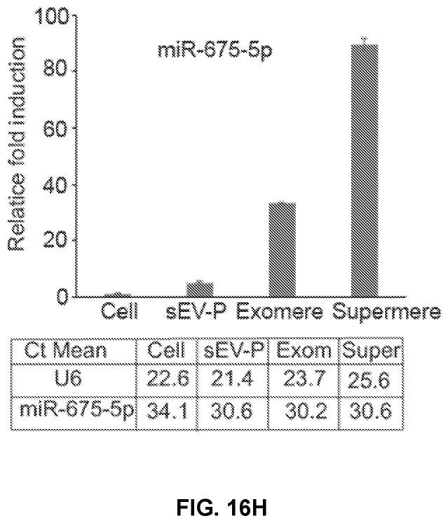

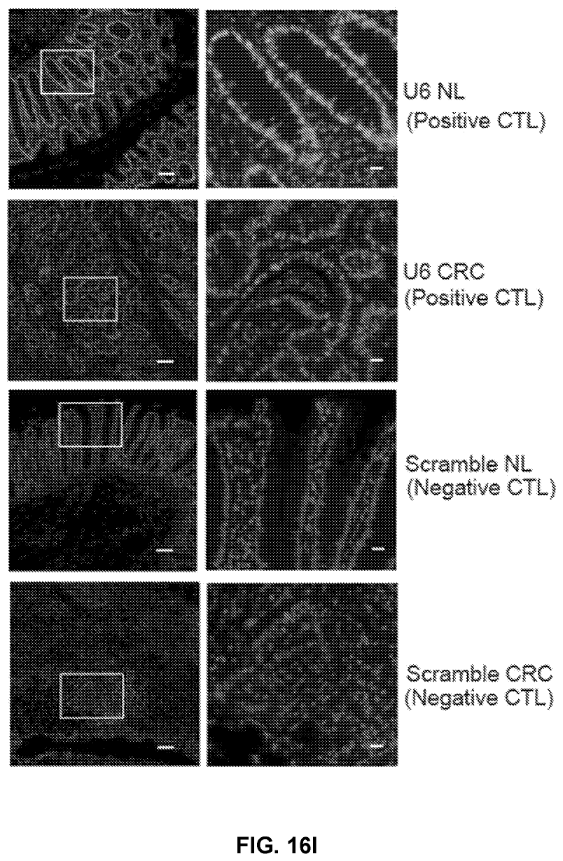

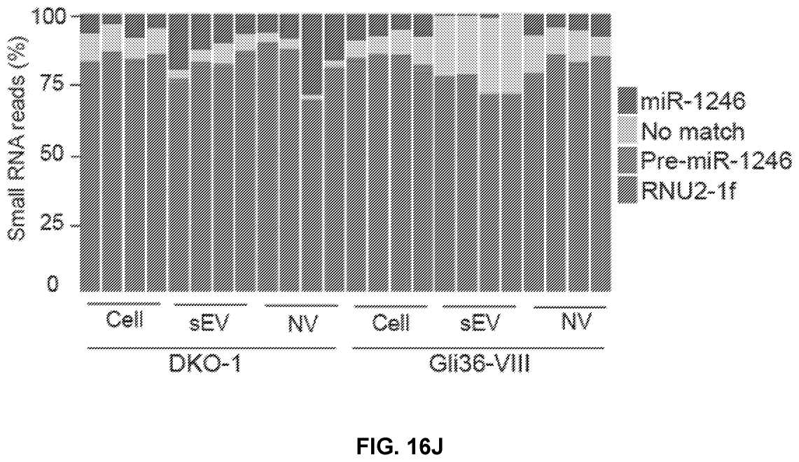

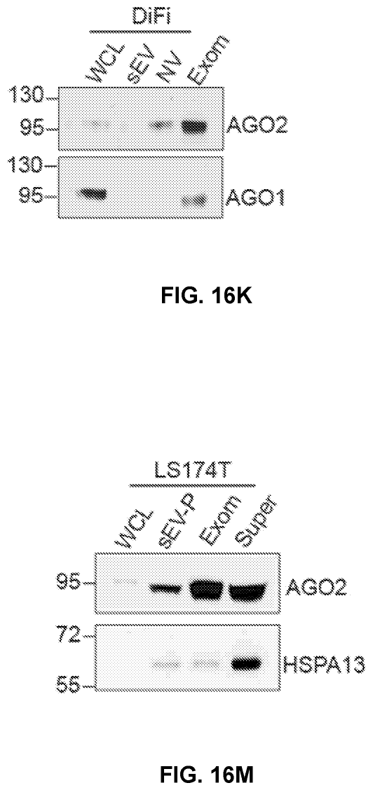

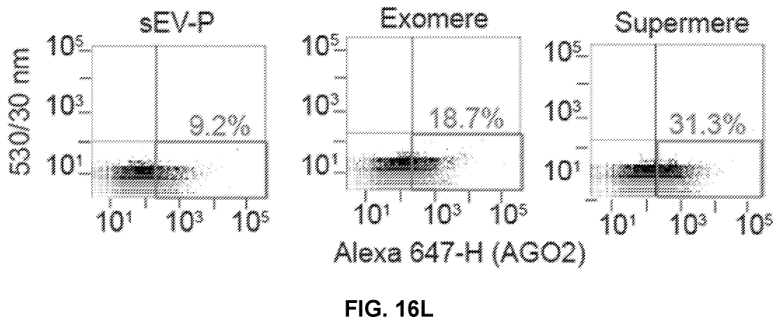

The RNA content in cells and extracellular carriers was next examined. EV-associated exRNAs, especially miRNAs, have attracted much attention due to their diverse biological functions and potential as cancer biomarkers (Jeppesen, D. K., et al. Cell 2019 177:428-445 e418; Srinivasan, S., et al. Cell 2019 177:446-462 e416; Murillo, O. D., et al. Cell 2019 177:463-477 e415; Slack, F. J. & Chinnaiyan, A. M. Cell 2019 179:1033-1055). The relative abundance of small exRNAs in supermeres was significantly higher than in exomeres and the sEV-P (FIG. 9A). The small exRNAs associated with DiFi cells and their extracellular compartments displayed distinct patterns of distribution (FIG. 16A, 16B). Amongst RNA populations, miRNAs were the dominant RNA species (FIG. 9B) with exomeres containing the highest relative level of miRNAs (79%). A high percentage of transfer RNA (IRNA) was a feature of cells and SEV-Ps (FIG. 9B,16C). Supermeres displayed a distinctive small exRNA repertoire with a relatively high percentage of snRNAs compared to exomeres, sEV-P and cells. Supermeres exhibited distinct miRNA profiles (FIG. 16D) and some miRNAs were detected solely in a specific extracellular compartment (FIG. 16E). The miRNA expression patterns of exomeres and supermeres were more closely related and distinct from cells and the sEV-P (FIG. 9C). Examination of the top 10 differently expressed miRNAs revealed that some miRNAs are mostly present in cells with limited secretion (FIG. 16F). The most highly abundant and enriched miRNAs in exomeres included miR-92a-3p, miR-1247-5p and miR-10a-5p (FIG. 9D, 16F, 16G). The most abundant and enriched miRNAs in supermeres included miR-1246 and miR-320b, c, d. miR-1246 and miR-675-5p were selectively enriched in supermeres compared to exomeres, while exomeres were enriched in miR-10a-5p and miR-1247-5p (FIG. 16G). The expression of two supermere-associated miRNAs, miR-1246 and miR-675, were validated (FIG. 9E,16H).

By far, the most abundant and most differentially expressed miRNA in supermeres was miR-1246 with a 210-fold change in expression levels compared to cells (FIG. 9D, 9E, 16G). Overexpression of miR-1246 has been observed in several cancer types, including lung and liver (Chai, S., et al. Hepatology 2016 64:2062-2076; Zhang, W. C., et al. Nat Commun 2016 7:11702). To further investigate the clinical relevance of miR-1246, fluorescence in situ hybridization (FISH) of miR-1246 in CRC tumour and normal tissues was performed on a tissue microarray (TMA). Expression of miR-1246 was predominantly nuclear in tumor and stromal cells (FIG. 9F, 16I). In normal epithelial cells, staining was either weak or undetectable, suggesting that miR-1246 is a potential biomarker for CRC. The strong nuclear miR-1246 staining in CRC tissue prompted us to explore the origin and biogenesis of miR-1246. The mature miR-1246 sequence overlaps completely with fragments of RNU2-1, a small nuclear RNA (snRNA) in the spliceosome, highlighting that miR-1246 could be a degradation product of RNU2-1 or originate from processing of pre-miR-1246 (Matera, A. G. & Wang, Z. Nat Rev Mol Cell Biol 2014 15:108-121). Many other snRNAs in supermeres are of the RNU2 pseudogene family and also contain miR-1246 sequences. Despite the divergence of sequences amongst RNU2 family members, the miR-1246 sequence is conserved in many family members. Furthermore, the majority of the miR-1246 sequences detected in both cells and extracellular compartments were derived from RNU2-1, not from the precursor miR-1246 (89% and 68% in the cells and supermeres, respectively) (FIG. 9G), consistent with previous data sets (FIG. 16J) and reports (Xu, Y. F., et al. RNA Biol 2019 16:770-784). However, the mature miR-1246 sequence was highly abundant in supermeres compared to cells (1800-fold) (FIG. 9G). Since the precursor miR-1246 was undetectable in both DiFi cells and extracellular compartments, this data supports miR-1246 being generated through a Drosha- and Dicer-independent pathway.

Several mechanisms have been proposed for sorting miRNAs into exosomes. The RNA-binding proteins, Y-box protein 1 (YBX1), sumoylated hnRNPA2B1 and Argonaute proteins (AGO1-4) have all been reported to mediate exosomal miRNA secretion (Shurtleff, M. J., et al. Elife 2016 5; Melo, S. A., et al. Cancer Cell 2014 26:707-721; Wu, B., et al. Nat Commun 2018 9:420). However, AGO1-4 are enriched in gradient-purified NV fractions and exomeres (Jeppesen, D. K., et al. Cell 2019 177:428-445 e418; Zhang, Q., et al. Cell Rep 2019 27:940-954 e946; Murillo, O. D., et al. Cell 2019 177:463-477 e415; Temoche-Diaz, M. M., et al. Elife 2019 8). The observed abundance of miRNAs in supermeres correlated with the proteomic data showing supermeres are highly enriched in ribonucleoproteins, including Argonaute proteins. AGO1 and AGO2 are highly enriched in DiFi cell-derived exomeres and supermeres but were not detected in high-resolution density gradient-purified sEVs (FIG. 9H, 16K). FAVS analysis confirmed that the expression level of AGO2 in DiFi supermeres is higher than in the sEV-P (FIG. 16L). AGO2 was also highly enriched in supermeres derived from PANC-1, SC and LS174T cells (FIG. 9J, 9K, 16M). CRC tissues displayed strong positive staining for AGO2 compared with adjacent normal colonic mucosa (FIG. 9L). Furthermore, the level of AGO2 detected by FAVS in supermeres and exomeres isolated from the plasma of CRC patients was higher than that from normal controls (FIG. 9M). Sumoylation of ribonucleoprotein hnRNPA2B1 has been attributed to miRNA sorting into exosomes, including sorting of miR-1246 (Villarroya-Beltri, C., et al. Nat Commun 2013 4:2980; Cooks, T., et al. Nat Commun 2018 9:771). However, hnRNPA2B1 was only detected in DiFi cells and supermeres (FIG. 9I), suggesting the possible involvement of hnRNPA2B1 in miRNA sorting can be attributed to supermeres. Exportin-5 (XPO5) exports pre-miRNA from the nucleus to the cytoplasm of cells (Mori, M. A., et al. Cell Metab 2019 30:656-673). XPO5 was enriched in extracellular NV fractions, exomeres and supermeres but was not detected in gradient-purified sEVs, suggesting that XPO5 may be involved in sorting of miRNAs to extracellular, non-vesicular, nanoparticles (FIG. 9H, 9I, 9K). Many known RNA-binding proteins (Hentze, M. W., et al. Nat Rev Mol Cell Biol 2018 19:327-341; Nussbacher, J. K. & Yeo, G. W. Mol Cell 2018 69:1005-1016 e1007) were found to be enriched specifically in NV fractions, exomeres and supermeres rather than sEVs (Table 1).

TABLE 1

| | SEV | SEV | SEV | NV | NV | NV | | | | | | |
|---|---|---|---|---|---|---|---|---|---|---|---|---|
| Gene name | A | B | C | A | B | C | Exomere A | Exomere B | Exomere C | Supermere A | Supermere B | Supermere C |
| AARS | 7 | 4 | 2 | 34 | 40 | 34 | 52 | 42 | 50 | 31 | 30 | 30 |
| ABCE1 | 2 | 4 | 2 | 14 | 16 | 13 | 16 | 10 | 9 | 4 | 0 | 4 |
| ABCF1 | 2 | 1 | 1 | 15 | 12 | 11 | 16 | 11 | 13 | 4 | 5 | 2 |
| ACAA2 | 1 | 0 | 1 | 7 | 7 | 5 | 39 | 33 | 39 | 123 | 108 | 110 |
| ACAT1 | 2 | 0 | 0 | 4 | 4 | 3 | 3 | 5 | 6 | 4 | 3 | 0 |
| ACO1 | 2 | 0 | 0 | 18 | 19 | 18 | 12 | 9 | 11 | 16 | 13 | 15 |
| ACO2 | 1 | 0 | 0 | 2 | 6 | 8 | 8 | 7 | 6 | 5 | 9 | 9 |
| ACTN4 | 35 | 39 | 30 | 50 | 56 | 39 | 112 | 102 | 105 | 456 | 460 | 436 |
| ADAR | 0 | 0 | 0 | 3 | 2 | 2 | 2 | 1 | 2 | 3 | 2 | 0 |
| ADD1 | 0 | 0 | 0 | 2 | 2 | 1 | 7 | 5 | 6 | 15 | 11 | 12 |
| ADD3 | 0 | 0 | 0 | 1 | 0 | 0 | 3 | 2 | 3 | 7 | 6 | 7 |
| ADK | 1 | 0 | 1 | 5 | 10 | 3 | 8 | 6 | 9 | 5 | 5 | 5 |
| AHNAK | 0 | 0 | 0 | 0 | 0 | 0 | 0 | 0 | 2 | 0 | 0 | 0 |
| AIMP1 | 0 | 0 | 0 | 4 | 4 | 4 | 4 | 3 | 5 | 0 | 2 | 0 |
| AKAP1 | 0 | 0 | 0 | 3 | 2 | 3 | 2 | 2 | 2 | 0 | 0 | 0 |
| AKAP8 | 0 | 0 | 0 | 0 | 0 | 0 | 0 | 0 | 0 | 0 | 2 | 0 |
| ALDH18A1 | 1 | 1 | 0 | 19 | 18 | 9 | 15 | 11 | 18 | 4 | 4 | 3 |
| ALDH7A1 | 1 | 0 | 0 | 6 | 6 | 3 | 3 | 4 | 7 | 7 | 7 | 6 |
| ALDOA | 12 | 9 | 5 | 16 | 16 | 17 | 58 | 69 | 61 | 388 | 334 | 349 |
| ALDOC | 3 | 3 | 4 | 5 | 5 | 4 | 13 | 11 | 13 | 46 | 36 | 34 |
| ANP32A | 0 | 0 | 0 | 0 | 0 | 0 | 0 | 0 | 0 | 4 | 6 | 6 |
| ANP32B | 0 | 0 | 0 | 0 | 0 | 0 | 0 | 0 | 0 | 0 | 3 | 0 |
| ANXA11 | 21 | 23 | 20 | 2 | 4 | 2 | 0 | 0 | 0 | 0 | 0 | 0 |
| ANXA2 | 34 | 38 | 34 | 6 | 7 | 9 | 3 | 4 | 1 | 8 | 2 | 3 |
| ANXA7 | 6 | 8 | 3 | 0 | 0 | 0 | 0 | 0 | 0 | 0 | 0 | 0 |
| APEH | 4 | 2 | 2 | 5 | 3 | 3 | 42 | 38 | 33 | 48 | 43 | 38 |
| APEX1 | 1 | 2 | 0 | 11 | 11 | 9 | 12 | 11 | 16 | 19 | 21 | 17 |
| API5 | 0 | 0 | 0 | 1 | 1 | 1 | 1 | 2 | 2 | 9 | 11 | 5 |
| AQR | 0 | 0 | 0 | 7 | 8 | 8 | 1 | 1 | 1 | 0 | 0 | 0 |
| ARCN1 | 0 | 0 | 0 | 3 | 5 | 1 | 5 | 3 | 2 | 2 | 4 | 3 |
| ARF1 | 23 | 25 | 29 | 12 | 14 | 11 | 13 | 11 | 9 | 12 | 7 | 8 |
| ARHGEF1 | 0 | 1 | 0 | 12 | 14 | 13 | 11 | 4 | 9 | 0 | 2 | 2 |
| ARHGEF2 | 0 | 0 | 0 | 3 | 1 | 1 | 0 | 0 | 0 | 0 | 0 | 0 |
| ARPC1B | 2 | 2 | 4 | 7 | 10 | 7 | 17 | 15 | 13 | 17 | 15 | 15 |
| ASS1 | 10 | 6 | 6 | 31 | 32 | 27 | 32 | 25 | 30 | 32 | 28 | 32 |
| ATP1A1 | 80 | 114 | 108 | 25 | 31 | 23 | 8 | 10 | 5 | 0 | 0 | 0 |
| ATP5A1 | 17 | 2 | 2 | 14 | 11 | 10 | 5 | 8 | 6 | 0 | 0 | 0 |
| ATP5B | 37 | 12 | 11 | 57 | 56 | 46 | 55 | 47 | 52 | 31 | 25 | 26 |
| ATXN2L | 0 | 0 | 0 | 0 | 1 | 0 | 3 | 0 | 1 | 0 | 0 | 0 |
| BCCIP | 0 | 0 | 0 | 1 | 1 | 1 | 2 | 1 | 1 | 2 | 4 | 4 |
| BOP1 | 0 | 0 | 0 | 6 | 7 | 4 | 1 | 1 | 2 | 0 | 0 | 0 |
| BRIX1 | 0 | 0 | 0 | 2 | 3 | 0 | 1 | 0 | 0 | 0 | 0 | 0 |
| BTF3 | 0 | 0 | 0 | 0 | 0 | 0 | 0 | 0 | 0 | 4 | 5 | 8 |
| BZW1 | 0 | 0 | 0 | 0 | 0 | 0 | 0 | 0 | 0 | 5 | 4 | 4 |
| BZW2 | 0 | 2 | 0 | 2 | 4 | 0 | 10 | 7 | 9 | 3 | 6 | 3 |
| C14orf166 | 1 | 1 | 1 | 2 | 3 | 2 | 0 | 2 | 2 | 2 | 2 | 2 |
| CACYBP | 0 | 0 | 0 | 3 | 1 | 0 | 1 | 3 | 2 | 4 | 3 | 3 |
| CALR | 0 | 1 | 0 | 1 | 2 | 0 | 2 | 3 | 1 | 35 | 37 | 34 |
| CAND1 | 4 | 6 | 4 | 33 | 34 | 26 | 68 | 53 | 65 | 74 | 54 | 61 |
| CAPRIN1 | 0 | 0 | 0 | 1 | 1 | 1 | 3 | 4 | 3 | 9 | 7 | 12 |
| CARHSP1 | 0 | 0 | 0 | 0 | 1 | 1 | 1 | 0 | 2 | 0 | 0 | 0 |
| CAT | 1 | 0 | 0 | 1 | 0 | 1 | 2 | 3 | 2 | 3 | 3 | 0 |
| CCAR1 | 0 | 0 | 0 | 2 | 1 | 1 | 4 | 4 | 4 | 2 | 2 | 0 |
| CCT2 | 17 | 11 | 9 | 54 | 51 | 37 | 53 | 44 | 58 | 35 | 38 | 35 |
| CCT3 | 11 | 11 | 7 | 38 | 40 | 36 | 48 | 36 | 47 | 33 | 39 | 38 |
| CCT4 | 9 | 7 | 3 | 28 | 26 | 25 | 37 | 28 | 35 | 26 | 26 | 25 |
| CCT6A | 7 | 3 | 2 | 30 | 25 | 22 | 37 | 25 | 31 | 35 | 34 | 35 |
| CD44 | 11 | 19 | 21 | 3 | 5 | 4 | 3 | 2 | 3 | 0 | 0 | 0 |
| CDC5L | 0 | 0 | 0 | 0 | 1 | 1 | 1 | 1 | 2 | 0 | 0 | 0 |
| CFL1 | 11 | 12 | 12 | 12 | 14 | 10 | 26 | 13 | 24 | 22 | 21 | 20 |
| CGN | 0 | 0 | 0 | 0 | 0 | 0 | 0 | 0 | 0 | 2 | 0 | 0 |
| CHERP | 0 | 0 | 0 | 2 | 6 | 1 | 1 | 3 | 3 | 2 | 2 | 3 |
| CHMP4B | 6 | 15 | 14 | 0 | 0 | 1 | 0 | 0 | 0 | 0 | 0 | 0 |
| CLNS1A | 0 | 0 | 0 | 2 | 2 | 1 | 2 | 2 | 3 | 0 | 0 | 0 |
| CLTC | 72 | 60 | 59 | 207 | 231 | 197 | 184 | 156 | 183 | 101 | 92 | 90 |
| CNDP2 | 4 | 5 | 2 | 25 | 23 | 16 | 22 | 18 | 21 | 37 | 41 | 39 |
| CNOT1 | 2 | 1 | 1 | 8 | 11 | 9 | 12 | 5 | 10 | 4 | 4 | 2 |
| COPB1 | 6 | 2 | 3 | 43 | 39 | 30 | 36 | 30 | 38 | 9 | 15 | 14 |
| COPG1 | 2 | 1 | 1 | 25 | 27 | 18 | 26 | 19 | 23 | 11 | 11 | 16 |
| CORO1A | 0 | 0 | 0 | 3 | 4 | 3 | 6 | 4 | 6 | 5 | 6 | 4 |
| CPNE3 | 8 | 10 | 8 | 0 | 1 | 1 | 0 | 0 | 0 | 0 | 0 | 0 |
| CPSF6 | 0 | 0 | 0 | 2 | 2 | 3 | 2 | 2 | 4 | 3 | 4 | 4 |
| CPSF7 | 0 | 0 | 0 | 0 | 4 | 3 | 1 | 2 | 2 | 0 | 0 | 0 |
| CRKL | 3 | 1 | 1 | 0 | 0 | 0 | 0 | 0 | 0 | 0 | 0 | 0 |
| CS | 3 | 3 | 1 | 20 | 18 | 14 | 19 | 12 | 17 | 10 | 14 | 9 |
| CSDE1 | 0 | 0 | 0 | 2 | 3 | 3 | 3 | 0 | 1 | 0 | 0 | 0 |
| CSE1L | 7 | 8 | 6 | 40 | 46 | 36 | 75 | 36 | 69 | 46 | 44 | 52 |
| CSTB | 1 | 2 | 1 | 1 | 1 | 1 | 1 | 2 | 1 | 0 | 5 | 2 |
| CSTF1 | 0 | 0 | 0 | 7 | 5 | 3 | 5 | 2 | 7 | 9 | 9 | 8 |
| CSTF3 | 0 | 0 | 0 | 4 | 3 | 2 | 4 | 5 | 4 | 8 | 5 | 7 |
| CTNNA1 | 1 | 2 | 2 | 1 | 0 | 1 | 2 | 2 | 1 | 6 | 5 | 7 |
| CTNND1 | 3 | 6 | 6 | 5 | 1 | 2 | 1 | 1 | 2 | 0 | 0 | 0 |
| CYCS | 0 | 0 | 0 | 0 | 0 | 0 | 0 | 0 | 0 | 0 | 0 | 2 |
| DARS | 9 | 8 | 6 | 31 | 36 | 32 | 36 | 31 | 32 | 11 | 7 | 8 |
| DAZAP1 | 0 | 0 | 0 | 0 | 0 | 0 | 0 | 0 | 0 | 0 | 2 | 0 |
| DBNL | 0 | 0 | 0 | 1 | 1 | 0 | 2 | 1 | 1 | 3 | 2 | 0 |
| DBR1 | 0 | 0 | 0 | 2 | 1 | 1 | 0 | 2 | 1 | 0 | 0 | 0 |
| DCD | 4 | 4 | 1 | 0 | 0 | 2 | 1 | 1 | 1 | 0 | 0 | 0 |
| DDX1 | 5 | 2 | 3 | 21 | 23 | 18 | 18 | 14 | 18 | 11 | 14 | 15 |
| DDX17 | 2 | 3 | 2 | 23 | 23 | 17 | 13 | 14 | 15 | 7 | 11 | 8 |
| DDX19A | 0 | 0 | 0 | 6 | 4 | 1 | 3 | 2 | 4 | 0 | 0 | 0 |
| DDX21 | 0 | 1 | 0 | 7 | 12 | 8 | 5 | 7 | 4 | 2 | 2 | 7 |
| DDX23 | 0 | 0 | 0 | 3 | 3 | 2 | 6 | 3 | 3 | 2 | 4 | 0 |
| DDX39A | 2 | 0 | 0 | 9 | 7 | 6 | 9 | 8 | 12 | 12 | 11 | 11 |
| DDX39B | 1 | 4 | 2 | 11 | 8 | 6 | 11 | 9 | 13 | 24 | 17 | 20 |
| DDX3X | 6 | 5 | 3 | 13 | 16 | 13 | 10 | 7 | 7 | 3 | 4 | 5 |
| DDX42 | 0 | 0 | 0 | 0 | 0 | 0 | 0 | 0 | 0 | 7 | 7 | 9 |
| DDX46 | 0 | 0 | 0 | 3 | 3 | 1 | 1 | 0 | 1 | 2 | 0 | 0 |
| DDX5 | 2 | 0 | 0 | 17 | 20 | 13 | 8 | 7 | 11 | 5 | 6 | 9 |
| DDX6 | 1 | 0 | 1 | 14 | 12 | 7 | 11 | 7 | 7 | 5 | 6 | 3 |
| DEK | 0 | 0 | 0 | 4 | 2 | 1 | 2 | 1 | 3 | 2 | 4 | 4 |
| DHX15 | 9 | 5 | 5 | 44 | 37 | 31 | 41 | 27 | 25 | 16 | 16 | 21 |
| DHX16 | 0 | 0 | 0 | 5 | 6 | 4 | 2 | 3 | 3 | 0 | 0 | 0 |
| DHX29 | 0 | 0 | 0 | 2 | 2 | 1 | 2 | 0 | 1 | 0 | 0 | 0 |
| DHX30 | 0 | 0 | 0 | 1 | 2 | 1 | 2 | 0 | 1 | 0 | 0 | 0 |
| DHX36 | 1 | 0 | 0 | 7 | 10 | 6 | 8 | 6 | 5 | 2 | 3 | 5 |
| DHX8 | 0 | 0 | 0 | 4 | 4 | 1 | 1 | 0 | 0 | 0 | 0 | 0 |
| DHX9 | 21 | 22 | 20 | 73 | 73 | 60 | 76 | 53 | 74 | 36 | 38 | 40 |
| DIAPH1 | 0 | 0 | 0 | 3 | 1 | 3 | 9 | 7 | 14 | 9 | 9 | 9 |
| DKC1 | 1 | 1 | 1 | 6 | 10 | 8 | 2 | 3 | 3 | 2 | 2 | 0 |
| DLD | 0 | 0 | 0 | 0 | 0 | 0 | 2 | 5 | 1 | 28 | 24 | 26 |
| DNAJA1 | 24 | 38 | 34 | 7 | 18 | 12 | 15 | 10 | 13 | 7 | 7 | 7 |
| DNAJC2 | 0 | 0 | 0 | 2 | 1 | 1 | 0 | 0 | 0 | 0 | 0 | 0 |
| DSP | 16 | 19 | 8 | 30 | 33 | 31 | 23 | 13 | 15 | 18 | 12 | 15 |
| DYNC1H1 | 31 | 21 | 11 | 176 | 179 | 147 | 126 | 98 | 111 | 14 | 11 | 10 |
| DYNC1I2 | 2 | 2 | 1 | 10 | 9 | 8 | 12 | 10 | 12 | 5 | 7 | 7 |
| DYNC1LI1 | 1 | 0 | 0 | 4 | 5 | 7 | 4 | 4 | 5 | 0 | 0 | 0 |
| EDC4 | 0 | 0 | 0 | 2 | 2 | 0 | 1 | 1 | 1 | 0 | 0 | 0 |
| EEF1A1 | 51 | 50 | 45 | 120 | 173 | 123 | 241 | 203 | 290 | 221 | 246 | 230 |
| EEF1G | 26 | 26 | 20 | 101 | 140 | 102 | 155 | 82 | 151 | 139 | 143 | 154 |
| EEF2 | 57 | 58 | 47 | 144 | 148 | 113 | 298 | 237 | 283 | 272 | 268 | 294 |
| EFTUD2 | 0 | 0 | 0 | 14 | 11 | 8 | 24 | 20 | 20 | 15 | 11 | 12 |
| EIF1AX | 0 | 0 | 0 | 2 | 0 | 0 | 0 | 0 | 1 | 0 | 0 | 0 |
| EIF2S1 | 5 | 6 | 4 | 20 | 19 | 16 | 16 | 18 | 16 | 15 | 15 | 15 |
| EIF2S2 | 1 | 0 | 0 | 8 | 6 | 6 | 11 | 4 | 9 | 0 | 0 | 2 |
| EIF3A | 10 | 5 | 3 | 29 | 29 | 26 | 39 | 30 | 36 | 16 | 9 | 13 |
| EIF3B | 3 | 2 | 2 | 18 | 23 | 14 | 27 | 18 | 24 | 22 | 23 | 29 |
| EIF3C | 7 | 7 | 2 | 21 | 23 | 17 | 29 | 28 | 35 | 10 | 9 | 11 |
| EIF3D | 3 | 1 | 0 | 21 | 16 | 15 | 20 | 16 | 16 | 7 | 8 | 7 |
| EIF3E | 5 | 4 | 2 | 14 | 14 | 10 | 31 | 30 | 20 | 6 | 6 | 6 |
| EIF3H | 1 | 1 | 0 | 9 | 10 | 7 | 19 | 14 | 15 | 5 | 5 | 2 |
| EIF3I | 1 | 2 | 2 | 11 | 16 | 13 | 13 | 12 | 7 | 2 | 3 | 4 |
| EIF3L | 7 | 6 | 2 | 22 | 19 | 13 | 30 | 19 | 21 | 15 | 10 | 10 |
| EIF4A1 | 11 | 12 | 6 | 40 | 55 | 41 | 33 | 30 | 31 | 9 | 10 | 10 |
| EIF4A2 | 9 | 0 | 6 | 20 | 24 | 13 | 13 | 15 | 13 | 6 | 7 | 0 |
| EIF4A3 | 10 | 6 | 4 | 33 | 39 | 27 | 33 | 27 | 30 | 15 | 9 | 10 |
| EIF4E | 0 | 1 | 1 | 1 | 2 | 4 | 2 | 1 | 3 | 0 | 0 | 0 |
| EIF4G1 | 0 | 0 | 0 | 5 | 4 | 3 | 1 | 0 | 4 | 8 | 11 | 9 |
| EIF4G2 | 0 | 0 | 0 | 0 | 0 | 0 | 0 | 0 | 0 | 0 | 0 | 3 |
| EIF5 | 1 | 0 | 0 | 4 | 3 | 4 | 2 | 4 | 3 | 4 | 4 | 4 |
| EIF5A | 4 | 1 | 2 | 6 | 4 | 4 | 7 | 7 | 5 | 7 | 5 | 10 |
| EIF5B | 0 | 0 | 0 | 2 | 3 | 2 | 5 | 7 | 2 | 13 | 15 | 12 |
| ELAC2 | 0 | 0 | 0 | 5 | 4 | 4 | 3 | 3 | 3 | 3 | 0 | 0 |
| EMG1 | 0 | 0 | 0 | 4 | 3 | 5 | 3 | 1 | 2 | 0 | 0 | 2 |
| EML4 | 0 | 2 | 0 | 21 | 28 | 17 | 32 | 24 | 30 | 13 | 15 | 15 |
| ENO1 | 31 | 30 | 28 | 49 | 46 | 32 | 126 | 97 | 132 | 454 | 454 | 476 |
| EPB41L2 | 10 | 11 | 8 | 12 | 14 | 12 | 9 | 6 | 9 | 4 | 0 | 2 |
| EPPK1 | 2 | 2 | 2 | 6 | 11 | 15 | 5 | 2 | 4 | 0 | 0 | 0 |
| EPRS | 15 | 13 | 9 | 73 | 72 | 64 | 83 | 61 | 82 | 34 | 33 | 37 |
| EPS8L3 | 18 | 32 | 29 | 1 | 1 | 0 | 0 | 0 | 0 | 0 | 0 | 0 |
| ERH | 0 | 0 | 0 | 0 | 0 | 0 | 0 | 0 | 0 | 0 | 2 | 4 |
| ERP29 | 0 | 0 | 0 | 0 | 0 | 0 | 0 | 0 | 0 | 16 | 10 | 11 |
| ETF1 | 0 | 0 | 0 | 5 | 7 | 2 | 1 | 4 | 3 | 4 | 4 | 4 |
| EWSR1 | 0 | 0 | 0 | 0 | 0 | 0 | 2 | 0 | 0 | 0 | 0 | 0 |
| EXOSC2 | 0 | 0 | 0 | 2 | 1 | 1 | 4 | 1 | 3 | 4 | 5 | 6 |
| EXOSC6 | 0 | 0 | 0 | 3 | 3 | 1 | 2 | 3 | 2 | 0 | 0 | 0 |
| EXOSC9 | 0 | 0 | 0 | 0 | 1 | 1 | 3 | 1 | 3 | 4 | 2 | 0 |
| EZR | 16 | 24 | 25 | 3 | 2 | 2 | 2 | 3 | 4 | 20 | 17 | 18 |
| F11R | 19 | 24 | 23 | 3 | 4 | 6 | 1 | 2 | 1 | 0 | 0 | 0 |
| FAM120A | 0 | 0 | 0 | 3 | 6 | 4 | 1 | 1 | 1 | 0 | 0 | 0 |
| FAM98A | 0 | 0 | 0 | 3 | 2 | 2 | 2 | 1 | 2 | 2 | 0 | 2 |
| FAM98B | 0 | 0 | 0 | 4 | 3 | 1 | 2 | 1 | 1 | 0 | 2 | 2 |
| FARSA | 1 | 1 | 0 | 17 | 18 | 16 | 5 | 4 | 5 | 0 | 0 | 0 |
| FARSB | 0 | 0 | 0 | 10 | 6 | 7 | 12 | 6 | 8 | 5 | 2 | 2 |
| FASN | 154 | 143 | 132 | 390 | 444 | 386 | 697 | 502 | 632 | 128 | 120 | 124 |
| FBL | 0 | 0 | 0 | 6 | 11 | 8 | 5 | 3 | 3 | 0 | 2 | 0 |
| FDPS | 0 | 0 | 0 | 2 | 0 | 1 | 11 | 8 | 9 | 24 | 24 | 26 |
| FEN1 | 1 | 0 | 0 | 3 | 3 | 2 | 2 | 2 | 1 | 3 | 0 | 4 |
| FKBP1A | 2 | 1 | 2 | 0 | 0 | 0 | 0 | 0 | 0 | 0 | 0 | 0 |
| FKBP4 | 2 | 0 | 0 | 13 | 14 | 9 | 13 | 12 | 16 | 25 | 22 | 20 |
| FLNA | 39 | 33 | 18 | 99 | 102 | 87 | 171 | 131 | 160 | 169 | 155 | 173 |
| FLNB | 40 | 19 | 14 | 100 | 105 | 92 | 219 | 181 | 252 | 311 | 308 | 307 |
| FN1 | 0 | 0 | 0 | 0 | 0 | 0 | 3 | 3 | 2 | 0 | 0 | 0 |
| FUBP1 | 0 | 0 | 0 | 0 | 0 | 0 | 0 | 0 | 0 | 4 | 3 | 0 |
| FUBP3 | 0 | 0 | 0 | 2 | 1 | 1 | 0 | 0 | 0 | 0 | 0 | 0 |
| G3BP1 | 1 | 0 | 0 | 6 | 6 | 3 | 3 | 3 | 3 | 4 | 7 | 6 |
| G3BP2 | 0 | 0 | 0 | 4 | 4 | 0 | 1 | 2 | 3 | 5 | 5 | 8 |
| GANAB | 25 | 17 | 17 | 66 | 79 | 68 | 98 | 79 | 80 | 61 | 59 | 59 |
| GAPDH | 60 | 57 | 47 | 270 | 375 | 299 | 333 | 261 | 334 | 181 | 168 | 173 |
| GDI2 | 17 | 20 | 17 | 10 | 8 | 5 | 7 | 8 | 8 | 57 | 62 | 55 |
| GEMIN5 | 0 | 0 | 0 | 2 | 2 | 2 | 4 | 4 | 4 | 0 | 0 | 0 |
| GGCT | 2 | 2 | 0 | 1 | 0 | 0 | 0 | 0 | 0 | 4 | 6 | 4 |
| GLRX3 | 0 | 0 | 0 | 0 | 0 | 0 | 0 | 0 | 0 | 2 | 2 | 4 |
| GMPR2 | 0 | 0 | 0 | 0 | 2 | 1 | 1 | 0 | 0 | 14 | 9 | 10 |
| GNE | 0 | 0 | 0 | 12 | 9 | 6 | 13 | 9 | 10 | 0 | 0 | 0 |
| GOT2 | 0 | 0 | 0 | 0 | 0 | 0 | 0 | 2 | 3 | 45 | 40 | 43 |
| GRB2 | 20 | 47 | 45 | 1 | 5 | 3 | 1 | 0 | 1 | 0 | 0 | 0 |
| GRN | 0 | 0 | 0 | 0 | 0 | 0 | 6 | 5 | 4 | 17 | 18 | 19 |
| GRWD1 | 0 | 0 | 0 | 8 | 5 | 5 | 7 | 4 | 5 | 2 | 2 | 2 |
| GSPT1 | 1 | 0 | 0 | 7 | 10 | 6 | 8 | 10 | 13 | 15 | 20 | 16 |
| GTPBP1 | 0 | 0 | 0 | 2 | 1 | 1 | 1 | 0 | 0 | 0 | 0 | 0 |
| H2AFY | 4 | 3 | 1 | 5 | 9 | 6 | 24 | 24 | 20 | 6 | 4 | 5 |
| HADH | 4 | 4 | 2 | 22 | 18 | 15 | 15 | 17 | 21 | 30 | 28 | 24 |
| HDAC2 | 3 | 0 | 0 | 19 | 17 | 13 | 14 | 12 | 16 | 9 | 7 | 8 |
| HDGF | 0 | 0 | 0 | 0 | 0 | 0 | 0 | 0 | 0 | 0 | 2 | 0 |
| HDLBP | 0 | 0 | 0 | 6 | 5 | 2 | 3 | 3 | 6 | 0 | 0 | 0 |
| HEATR1 | 0 | 0 | 0 | 4 | 6 | 1 | 3 | 1 | 2 | 0 | 0 | 0 |
| HIBADH | 0 | 0 | 0 | 0 | 0 | 0 | 2 | 0 | 1 | 8 | 7 | 4 |
| HIST1H1B | 0 | 0 | 0 | 0 | 3 | 1 | 8 | 4 | 4 | 0 | 0 | 0 |
| HIST1H1C | 3 | 2 | 1 | 1 | 4 | 4 | 14 | 13 | 6 | 0 | 0 | 0 |
| HIST1H1E | 3 | 2 | 1 | 1 | 5 | 4 | 14 | 13 | 6 | 0 | 0 | 0 |
| HIST1H4A | 14 | 17 | 10 | 14 | 21 | 18 | 47 | 60 | 34 | 14 | 14 | 17 |
| HLA-A | 34 | 43 | 43 | 16 | 27 | 28 | 24 | 17 | 19 | 24 | 21 | 22 |
| HMGB1 | 0 | 1 | 1 | 2 | 3 | 2 | 2 | 2 | 2 | 24 | 18 | 21 |
| HMGB2 | 0 | 0 | 0 | 0 | 0 | 0 | 0 | 0 | 0 | 4 | 0 | 3 |
| HNRNPA1 | 0 | 0 | 0 | 2 | 2 | 2 | 2 | 1 | 2 | 12 | 9 | 7 |
| HNRNPA2B1 | 0 | 3 | 1 | 0 | 1 | 1 | 1 | 1 | 1 | 9 | 8 | 8 |
| HNRNPA3 | 0 | 0 | 0 | 3 | 6 | 3 | 3 | 1 | 2 | 0 | 0 | 0 |
| HNRNPAB | 1 | 1 | 1 | 2 | 2 | 1 | 4 | 3 | 3 | 7 | 5 | 5 |
| HNRNPC | 2 | 2 | 0 | 4 | 5 | 2 | 7 | 9 | 5 | 7 | 8 | 10 |
| HNRNPD | 3 | 3 | 2 | 8 | 10 | 6 | 17 | 11 | 13 | 14 | 16 | 17 |
| HNRNPF | 3 | 5 | 2 | 14 | 14 | 8 | 9 | 10 | 12 | 21 | 20 | 18 |
| HNRNPH2 | 2 | 1 | 1 | 8 | 6 | 6 | 1 | 2 | 0 | 0 | 0 | 0 |
| HNRNPK | 10 | 7 | 3 | 20 | 23 | 16 | 24 | 17 | 24 | 25 | 25 | 28 |
| HNRNPL | 4 | 4 | 2 | 20 | 23 | 15 | 33 | 27 | 32 | 35 | 32 | 34 |
| HNRNPM | 0 | 0 | 0 | 3 | 3 | 5 | 4 | 2 | 1 | 3 | 2 | 0 |
| HNRNPR | 2 | 2 | 2 | 6 | 4 | 2 | 6 | 6 | 4 | 16 | 19 | 19 |
| HNRNPU | 3 | 3 | 2 | 21 | 20 | 12 | 24 | 18 | 22 | 19 | 23 | 19 |
| HNRNPUL1 | 0 | 0 | 0 | 14 | 18 | 14 | 9 | 8 | 5 | 3 | 4 | 3 |
| HNRNPUL2 | 3 | 1 | 2 | 22 | 25 | 22 | 26 | 17 | 24 | 16 | 18 | 13 |
| HOOK1 | 0 | 0 | 0 | 1 | 1 | 1 | 2 | 0 | 1 | 0 | 0 | 0 |
| HSD17B10 | 0 | 0 | 0 | 6 | 5 | 7 | 7 | 4 | 7 | 6 | 6 | 2 |
| HSP90AA1 | 57 | 55 | 45 | 172 | 192 | 112 | 385 | 326 | 435 | 340 | 405 | 396 |
| HSP90AB1 | 57 | 63 | 45 | 186 | 208 | 127 | 401 | 343 | 449 | 326 | 363 | 390 |
| HSP90B1 | 2 | 3 | 3 | 11 | 9 | 8 | 27 | 17 | 28 | 49 | 53 | 45 |
| HSPA1A | 46 | 61 | 53 | 34 | 39 | 24 | 78 | 66 | 76 | 128 | 150 | 162 |
| HSPA5 | 7 | 10 | 8 | 14 | 16 | 11 | 40 | 32 | 37 | 50 | 66 | 63 |
| HSPA8 | 80 | 108 | 120 | 112 | 86 | 66 | 218 | 193 | 264 | 305 | 392 | 384 |
| HSPA9 | 1 | 0 | 0 | 8 | 4 | 3 | 9 | 11 | 11 | 7 | 7 | 10 |
| HSPB1 | 0 | 4 | 0 | 0 | 0 | 1 | 0 | 0 | 0 | 0 | 0 | 0 |
| HSPD1 | 16 | 5 | 3 | 36 | 37 | 29 | 48 | 46 | 50 | 94 | 89 | 95 |
| HSPE1 | 0 | 0 | 0 | 0 | 0 | 0 | 3 | 3 | 1 | 13 | 13 | 14 |
| HUWE1 | 1 | 1 | 2 | 30 | 25 | 15 | 49 | 35 | 50 | 5 | 7 | 5 |
| IDH1 | 18 | 13 | 6 | 26 | 31 | 22 | 62 | 43 | 60 | 130 | 133 | 121 |
| IDH2 | 6 | 4 | 3 | 8 | 13 | 8 | 12 | 11 | 12 | 8 | 8 | 7 |
| IGF2BP2 | 0 | 0 | 0 | 1 | 2 | 0 | 0 | 0 | 0 | 0 | 0 | 0 |
| ILF2 | 4 | 5 | 2 | 12 | 12 | 9 | 22 | 17 | 22 | 59 | 57 | 60 |
| ILF3 | 0 | 0 | 0 | 5 | 4 | 3 | 7 | 6 | 6 | 31 | 34 | 23 |
| IPO4 | 1 | 1 | 0 | 6 | 4 | 4 | 7 | 7 | 5 | 4 | 3 | 2 |
| IPO5 | 7 | 9 | 7 | 39 | 48 | 38 | 97 | 63 | 95 | 93 | 77 | 79 |
| IREB2 | 0 | 0 | 0 | 0 | 0 | 0 | 0 | 0 | 0 | 0 | 3 | 0 |
| KHDRBS1 | 2 | 1 | 0 | 6 | 8 | 5 | 2 | 2 | 3 | 4 | 3 | 3 |
| KHSRP | 0 | 0 | 0 | 1 | 1 | 2 | 5 | 2 | 3 | 7 | 4 | 8 |
| KIAA1429 | 0 | 0 | 0 | 0 | 0 | 0 | 2 | 1 | 2 | 4 | 3 | 3 |
| KPNA2 | 0 | 1 | 0 | 8 | 8 | 6 | 7 | 8 | 7 | 8 | 8 | 12 |
| KPNB1 | 8 | 10 | 7 | 46 | 48 | 40 | 94 | 66 | 91 | 86 | 75 | 75 |
| KRT18 | 18 | 8 | 7 | 28 | 31 | 27 | 41 | 31 | 50 | 150 | 187 | 171 |
| KTN1 | 0 | 0 | 0 | 0 | 0 | 0 | 0 | 0 | 0 | 8 | 7 | 7 |
| LAMA2 | 0 | 0 | 0 | 0 | 0 | 0 | 10 | 13 | 5 | 4 | 4 | 6 |
| LASP1 | 0 | 2 | 1 | 1 | 0 | 0 | 1 | 1 | 0 | 0 | 0 | 0 |
| LDHA | 6 | 6 | 6 | 11 | 16 | 9 | 88 | 64 | 88 | 377 | 354 | 347 |
| LDHB | 15 | 12 | 9 | 15 | 17 | 15 | 87 | 57 | 76 | 319 | 332 | 326 |
| LGALS3 | 7 | 9 | 12 | 5 | 6 | 8 | 10 | 10 | 10 | 24 | 32 | 32 |
| LIN7C | 9 | 11 | 12 | 0 | 0 | 0 | 0 | 0 | 0 | 0 | 0 | 0 |
| LMNA | 1 | 4 | 0 | 10 | 10 | 9 | 25 | 20 | 24 | 35 | 39 | 40 |
| LRP1 | 0 | 0 | 0 | 0 | 0 | 0 | 7 | 6 | 5 | 0 | 0 | 0 |
| LRPPRC | 0 | 0 | 0 | 9 | 9 | 8 | 16 | 9 | 16 | 2 | 2 | 3 |
| LRRC47 | 0 | 0 | 0 | 4 | 3 | 3 | 3 | 4 | 3 | 0 | 0 | 0 |
| LSM2 | 0 | 0 | 0 | 3 | 1 | 0 | 2 | 1 | 2 | 0 | 0 | 0 |
| LTA4H | 0 | 0 | 0 | 0 | 1 | 0 | 2 | 2 | 1 | 34 | 33 | 36 |
| LUC7L2 | 0 | 0 | 0 | 0 | 0 | 0 | 0 | 0 | 0 | 2 | 2 | 5 |
| MACF1 | 0 | 0 | 0 | 2 | 2 | 1 | 3 | 4 | 6 | 6 | 3 | 4 |
| MAGOH | 0 | 0 | 0 | 0 | 0 | 0 | 0 | 0 | 0 | 2 | 3 | 2 |
| MAPRE1 | 0 | 0 | 0 | 2 | 1 | 0 | 0 | 0 | 1 | 0 | 3 | 4 |
| MARK2 | 0 | 2 | 2 | 1 | 1 | 0 | 0 | 0 | 0 | 0 | 0 | 0 |
| MAT2A | 1 | 0 | 0 | 9 | 9 | 6 | 8 | 9 | 11 | 11 | 12 | 10 |
| MATR3 | 1 | 0 | 0 | 7 | 3 | 1 | 3 | 3 | 2 | 2 | 4 | 2 |
| MCAT | 0 | 0 | 0 | 1 | 2 | 1 | 0 | 0 | 0 | 0 | 0 | 0 |
| MDH1 | 4 | 1 | 0 | 0 | 0 | 0 | 1 | 0 | 0 | 53 | 57 | 48 |
| MDH2 | 5 | 0 | 0 | 9 | 10 | 6 | 10 | 8 | 14 | 49 | 45 | 45 |
| METAP2 | 0 | 0 | 0 | 7 | 4 | 3 | 6 | 4 | 6 | 7 | 9 | 12 |
| MOV10 | 0 | 0 | 0 | 4 | 7 | 4 | 5 | 4 | 5 | 0 | 0 | 0 |
| MTHFD1 | 14 | 18 | 15 | 57 | 60 | 44 | 53 | 45 | 43 | 15 | 20 | 26 |
| MYBBP1A | 0 | 0 | 0 | 2 | 1 | 1 | 2 | 0 | 0 | 0 | 0 | 0 |
| MYH14 | 28 | 16 | 15 | 96 | 95 | 87 | 86 | 78 | 78 | 59 | 43 | 38 |
| MYH9 | 49 | 44 | 24 | 132 | 132 | 106 | 170 | 134 | 179 | 175 | 162 | 162 |
| MYO18A | 0 | 0 | 0 | 7 | 6 | 4 | 8 | 4 | 6 | 0 | 0 | 0 |
| NAA15 | 1 | 0 | 0 | 10 | 13 | 8 | 13 | 8 | 8 | 5 | 6 | 3 |
| NAA50 | 0 | 0 | 0 | 2 | 3 | 0 | 3 | 1 | 0 | 0 | 0 | 0 |
| NAP1L1 | 0 | 0 | 0 | 1 | 2 | 0 | 7 | 3 | 3 | 4 | 5 | 5 |
| NAP1L4 | 0 | 0 | 0 | 4 | 5 | 4 | 9 | 7 | 7 | 10 | 10 | 9 |
| NARS | 3 | 6 | 3 | 28 | 26 | 19 | 43 | 33 | 39 | 22 | 27 | 23 |
| NAT10 | 0 | 2 | 0 | 9 | 9 | 7 | 4 | 5 | 5 | 0 | 0 | 0 |
| NCBP1 | 2 | 2 | 1 | 23 | 20 | 20 | 30 | 16 | 25 | 17 | 15 | 13 |
| NCBP2 | 0 | 0 | 0 | 1 | 0 | 1 | 2 | 1 | 2 | 0 | 0 | 0 |
| NCL | 0 | 0 | 0 | 1 | 1 | 1 | 11 | 8 | 6 | 40 | 45 | 39 |
| NCOA5 | 0 | 0 | 0 | 0 | 0 | 0 | 0 | 0 | 0 | 0 | 2 | 0 |
| NDRG1 | 5 | 3 | 3 | 1 | 0 | 0 | 0 | 0 | 2 | 4 | 4 | 3 |
| NMD3 | 0 | 0 | 0 | 3 | 3 | 0 | 1 | 1 | 0 | 0 | 0 | 0 |
| NME1 | 0 | 0 | 0 | 0 | 0 | 0 | 0 | 0 | 0 | 33 | 35 | 35 |
| NMT1 | 1 | 1 | 0 | 14 | 13 | 11 | 4 | 4 | 3 | 2 | 2 | 2 |
| NOC4L | 0 | 0 | 0 | 2 | 4 | 1 | 0 | 0 | 0 | 0 | 0 | 0 |
| NOL11 | 0 | 0 | 0 | 4 | 6 | 3 | 5 | 1 | 1 | 2 | 0 | 0 |
| NOL6 | 0 | 0 | 0 | 4 | 2 | 5 | 2 | 0 | 0 | 0 | 0 | 0 |
| NONO | 10 | 12 | 9 | 27 | 25 | 26 | 20 | 18 | 25 | 11 | 11 | 13 |
| NOP2 | 1 | 0 | 1 | 3 | 3 | 3 | 3 | 3 | 2 | 0 | 0 | 0 |
| NOP56 | 2 | 1 | 1 | 15 | 18 | 14 | 15 | 11 | 18 | 0 | 0 | 0 |
| NOP58 | 1 | 1 | 2 | 8 | 9 | 7 | 10 | 7 | 6 | 5 | 3 | 2 |
| NPM1 | 0 | 0 | 0 | 4 | 4 | 4 | 20 | 25 | 18 | 25 | 21 | 22 |
| NPM3 | 0 | 0 | 0 | 0 | 0 | 0 | 0 | 0 | 0 | 2 | 2 | 3 |
| NQO1 | 10 | 8 | 9 | 23 | 29 | 18 | 17 | 14 | 16 | 6 | 7 | 7 |
| NSF | 0 | 0 | 0 | 8 | 9 | 2 | 7 | 3 | 4 | 0 | 0 | 0 |
| NSFL1C | 0 | 0 | 0 | 0 | 0 | 0 | 6 | 4 | 5 | 8 | 5 | 6 |
| NSUN2 | 0 | 0 | 0 | 5 | 13 | 8 | 7 | 2 | 4 | 0 | 0 | 0 |
| NUDT16L1 | 0 | 0 | 0 | 0 | 2 | 1 | 0 | 0 | 1 | 0 | 0 | 0 |
| NUDT21 | 0 | 1 | 0 | 4 | 2 | 2 | 5 | 5 | 5 | 14 | 17 | 19 |
| NXF1 | 0 | 0 | 0 | 5 | 4 | 4 | 3 | 2 | 5 | 0 | 0 | 0 |
| OLA1 | 1 | 1 | 1 | 9 | 6 | 3 | 7 | 4 | 8 | 15 | 18 | 15 |
| P4HB | 2 | 4 | 2 | 16 | 14 | 8 | 39 | 31 | 44 | 83 | 96 | 91 |
| PA2G4 | 3 | 3 | 1 | 9 | 9 | 7 | 24 | 17 | 26 | 50 | 45 | 49 |
| PABPC1 | 9 | 7 | 3 | 4 | 5 | 4 | 24 | 23 | 22 | 20 | 16 | 26 |
| PABPC4 | 0 | 0 | 0 | 0 | 0 | 0 | 0 | 0 | 0 | 8 | 0 | 9 |
| PAFAH1B3 | 1 | 1 | 0 | 1 | 2 | 2 | 3 | 2 | 1 | 9 | 9 | 11 |
| PAICS | 9 | 8 | 7 | 18 | 22 | 19 | 45 | 41 | 43 | 41 | 35 | 35 |
| PAIP1 | 0 | 1 | 0 | 0 | 0 | 0 | 2 | 1 | 2 | 4 | 6 | 3 |
| PARK7 | 0 | 0 | 0 | 0 | 0 | 0 | 0 | 0 | 0 | 13 | 13 | 11 |
| PARP1 | 0 | 0 | 0 | 5 | 4 | 1 | 4 | 3 | 3 | 6 | 9 | 5 |
| PCBP1 | 6 | 8 | 6 | 15 | 12 | 11 | 14 | 13 | 13 | 21 | 21 | 18 |
| PCBP2 | 4 | 4 | 3 | 10 | 8 | 7 | 11 | 9 | 9 | 11 | 11 | 12 |
| PCSK9 | 10 | 6 | 4 | 32 | 33 | 26 | 70 | 64 | 65 | 188 | 189 | 216 |
| PDCD6IP | 156 | 228 | 261 | 51 | 60 | 58 | 62 | 52 | 50 | 27 | 35 | 35 |
| PDIA3 | 1 | 1 | 1 | 7 | 5 | 3 | 12 | 9 | 13 | 62 | 69 | 67 |
| PDIA4 | 0 | 0 | 0 | 0 | 0 | 0 | 8 | 7 | 7 | 60 | 59 | 50 |
| PDLIM5 | 0 | 0 | 0 | 1 | 1 | 0 | 2 | 1 | 0 | 0 | 0 | 0 |
| PDS5A | 0 | 0 | 0 | 3 | 4 | 1 | 1 | 0 | 1 | 0 | 0 | 0 |
| PEBP1 | 8 | 8 | 7 | 1 | 0 | 0 | 1 | 0 | 1 | 10 | 7 | 11 |
| PEF1 | 3 | 3 | 2 | 0 | 1 | 1 | 0 | 1 | 1 | 0 | 0 | 0 |
| PFN1 | 12 | 14 | 13 | 13 | 10 | 10 | 9 | 13 | 9 | 16 | 14 | 17 |
| PHF5A | 0 | 0 | 0 | 1 | 1 | 0 | 2 | 2 | 2 | 0 | 0 | 0 |
| PLEC | 16 | 9 | 4 | 134 | 130 | 100 | 113 | 87 | 122 | 89 | 91 | 93 |
| POLR2A | 0 | 0 | 0 | 15 | 17 | 11 | 17 | 14 | 16 | 0 | 0 | 0 |
| POLR2B | 0 | 0 | 0 | 12 | 10 | 6 | 18 | 11 | 15 | 0 | 0 | 0 |
| POP1 | 0 | 0 | 0 | 2 | 1 | 0 | 2 | 0 | 1 | 0 | 0 | 0 |
| PPIB | 0 | 0 | 0 | 0 | 0 | 0 | 0 | 0 | 0 | 6 | 9 | 7 |
| PPID | 0 | 0 | 0 | 1 | 0 | 0 | 0 | 2 | 1 | 0 | 0 | 0 |
| PRDX1 | 10 | 9 | 7 | 9 | 12 | 10 | 18 | 16 | 22 | 36 | 40 | 34 |
| PRDX3 | 0 | 0 | 0 | 0 | 0 | 0 | 0 | 0 | 0 | 9 | 9 | 10 |
| PRKAR2A | 3 | 2 | 2 | 16 | 14 | 15 | 17 | 14 | 17 | 11 | 8 | 8 |
| PRKDC | 1 | 3 | 1 | 71 | 93 | 64 | 18 | 10 | 12 | 0 | 0 | 0 |
| PRKRA | 0 | 0 | 0 | 2 | 0 | 0 | 0 | 0 | 0 | 0 | 0 | 0 |
| PRMT1 | 12 | 10 | 8 | 29 | 38 | 32 | 34 | 27 | 30 | 13 | 18 | 14 |
| PRPF31 | 0 | 0 | 0 | 2 | 1 | 2 | 1 | 1 | 1 | 0 | 0 | 0 |
| PRPF4B | 0 | 0 | 0 | 2 | 2 | 1 | 1 | 1 | 0 | 0 | 0 | 0 |
| PRPF8 | 2 | 2 | 1 | 19 | 20 | 11 | 24 | 21 | 16 | 4 | 4 | 4 |
| PSMA4 | 4 | 4 | 1 | 2 | 3 | 0 | 37 | 42 | 39 | 7 | 6 | 7 |
| PSMB1 | 5 | 7 | 9 | 2 | 3 | 3 | 46 | 65 | 47 | 5 | 0 | 0 |
| PSMC1 | 7 | 5 | 5 | 20 | 23 | 21 | 16 | 12 | 21 | 3 | 0 | 0 |
| PSMC2 | 5 | 7 | 4 | 31 | 27 | 27 | 27 | 22 | 29 | 0 | 0 | 0 |
| PSPC1 | 1 | 1 | 0 | 6 | 6 | 4 | 5 | 5 | 6 | 8 | 6 | 4 |
| PTBP1 | 3 | 2 | 2 | 4 | 3 | 2 | 5 | 4 | 5 | 31 | 22 | 28 |
| PTPN1 | 0 | 0 | 0 | 3 | 3 | 2 | 4 | 2 | 4 | 0 | 0 | 0 |
| PTPN12 | 0 | 0 | 0 | 3 | 3 | 2 | 1 | 1 | 0 | 0 | 0 | 0 |
| PUF60 | 0 | 0 | 0 | 0 | 0 | 0 | 0 | 0 | 0 | 6 | 6 | 10 |
| PURA | 0 | 0 | 0 | 0 | 0 | 0 | 4 | 3 | 1 | 5 | 6 | 4 |
| PURB | 0 | 0 | 0 | 0 | 0 | 0 | 5 | 4 | 3 | 5 | 7 | 4 |
| PUS1 | 0 | 0 | 0 | 2 | 1 | 0 | 0 | 0 | 0 | 0 | 0 | 0 |
| PUS7 | 1 | 1 | 0 | 6 | 5 | 1 | 5 | 5 | 3 | 3 | 5 | 5 |
| RALY | 0 | 0 | 0 | 3 | 2 | 3 | 3 | 1 | 1 | 0 | 0 | 0 |
| RAN | 12 | 14 | 9 | 18 | 25 | 17 | 18 | 19 | 19 | 13 | 13 | 15 |
| RANBP2 | 0 | 0 | 0 | 8 | 7 | 2 | 10 | 7 | 9 | 0 | 0 | 0 |
| RBBP5 | 0 | 0 | 0 | 1 | 2 | 2 | 1 | 2 | 3 | 0 | 0 | 0 |
| RBM14 | 0 | 0 | 1 | 2 | 2 | 1 | 1 | 1 | 1 | 0 | 0 | 0 |
| RBM25 | 0 | 0 | 0 | 0 | 0 | 0 | 1 | 2 | 2 | 0 | 0 | 0 |
| RBM39 | 0 | 0 | 0 | 2 | 2 | 1 | 2 | 1 | 2 | 0 | 0 | 0 |
| RBM4 | 0 | 0 | 0 | 2 | 2 | 0 | 0 | 0 | 1 | 0 | 0 | 0 |
| RCC2 | 5 | 3 | 3 | 20 | 21 | 14 | 19 | 16 | 20 | 23 | 17 | 16 |
| REPIN1 | 0 | 0 | 0 | 1 | 1 | 2 | 2 | 2 | 2 | 0 | 0 | 0 |
| RNH1 | 2 | 2 | 3 | 4 | 4 | 5 | 9 | 5 | 6 | 21 | 16 | 18 |
| RPL10 | 1 | 2 | 2 | 12 | 10 | 7 | 8 | 5 | 6 | 0 | 0 | 0 |
| RPL10A | 8 | 15 | 12 | 11 | 10 | 7 | 9 | 8 | 12 | 16 | 16 | 15 |
| RPL11 | 2 | 1 | 2 | 3 | 4 | 3 | 1 | 1 | 2 | 0 | 0 | 0 |
| RPL12 | 6 | 5 | 4 | 10 | 8 | 7 | 12 | 9 | 11 | 10 | 10 | 11 |
| RPL13 | 0 | 0 | 0 | 2 | 2 | 0 | 0 | 1 | 2 | 0 | 0 | 0 |
| RPL13A | 1 | 0 | 0 | 2 | 3 | 0 | 0 | 0 | 1 | 0 | 0 | 0 |
| RPL14 | 0 | 0 | 0 | 5 | 7 | 5 | 3 | 0 | 3 | 0 | 0 | 0 |
| RPL17 | 1 | 0 | 0 | 7 | 6 | 5 | 4 | 6 | 3 | 0 | 0 | 2 |
| RPL18 | 3 | 3 | 3 | 2 | 5 | 5 | 2 | 1 | 2 | 0 | 0 | 0 |
| RPL18A | 2 | 1 | 1 | 5 | 7 | 3 | 1 | 2 | 2 | 0 | 0 | 0 |
| RPL19 | 0 | 0 | 0 | 1 | 1 | 1 | 2 | 1 | 0 | 0 | 0 | 0 |
| RPL21 | 1 | 1 | 1 | 3 | 4 | 3 | 2 | 1 | 2 | 0 | 0 | 0 |
| RPL22 | 0 | 1 | 1 | 5 | 5 | 5 | 5 | 5 | 5 | 5 | 4 | 3 |
| RPL23 | 1 | 2 | 1 | 5 | 4 | 3 | 3 | 3 | 5 | 0 | 0 | 0 |
| RPL24 | 0 | 0 | 0 | 2 | 2 | 2 | 1 | 0 | 0 | 0 | 0 | 0 |
| RPL27 | 0 | 0 | 0 | 3 | 3 | 3 | 3 | 2 | 2 | 0 | 0 | 0 |
| RPL27A | 2 | 2 | 2 | 4 | 4 | 3 | 2 | 1 | 0 | 0 | 0 | 0 |
| RPL28 | 0 | 0 | 0 | 2 | 2 | 1 | 3 | 2 | 2 | 0 | 0 | 0 |
| RPL3 | 0 | 0 | 0 | 5 | 5 | 6 | 4 | 3 | 4 | 0 | 2 | 2 |
| RPL30 | 1 | 1 | 0 | 5 | 5 | 4 | 4 | 5 | 3 | 4 | 4 | 3 |
| RPL31 | 0 | 0 | 0 | 2 | 2 | 1 | 0 | 1 | 0 | 0 | 0 | 0 |
| RPL32 | 1 | 0 | 0 | 0 | 2 | 1 | 1 | 1 | 1 | 0 | 0 | 0 |
| RPL35A | 0 | 0 | 0 | 2 | 1 | 1 | 2 | 1 | 2 | 0 | 0 | 0 |
| RPL38 | 1 | 0 | 0 | 1 | 4 | 2 | 3 | 1 | 2 | 2 | 0 | 0 |
| RPL4 | 3 | 2 | 2 | 12 | 14 | 12 | 1 | 3 | 1 | 0 | 0 | 0 |
| RPL5 | 3 | 1 | 0 | 18 | 16 | 13 | 7 | 4 | 3 | 5 | 2 | 0 |
| RPL6 | 0 | 0 | 0 | 6 | 8 | 5 | 2 | 0 | 0 | 0 | 0 | 0 |
| RPL7 | 1 | 3 | 1 | 5 | 9 | 4 | 2 | 0 | 2 | 0 | 0 | 0 |
| RPL7A | 2 | 0 | 0 | 11 | 12 | 7 | 2 | 1 | 0 | 0 | 0 | 0 |
| RPL8 | 0 | 0 | 0 | 2 | 3 | 4 | 1 | 1 | 1 | 0 | 0 | 0 |
| RPLP0 | 9 | 7 | 8 | 22 | 20 | 22 | 29 | 23 | 26 | 23 | 26 | 25 |
| RPLP2 | 2 | 0 | 0 | 10 | 8 | 5 | 15 | 14 | 9 | 6 | 7 | 5 |
| RPP30 | 0 | 0 | 0 | 3 | 5 | 1 | 3 | 3 | 3 | 0 | 0 | 0 |
| RPRD1B | 0 | 0 | 0 | 1 | 0 | 0 | 2 | 1 | 1 | 3 | 4 | 3 |
| RPRD2 | 0 | 0 | 0 | 2 | 2 | 0 | 2 | 2 | 0 | 0 | 0 | 0 |
| RPS10 | 1 | 1 | 1 | 3 | 3 | 3 | 1 | 1 | 1 | 0 | 0 | 0 |
| RPS11 | 3 | 4 | 3 | 14 | 13 | 13 | 10 | 5 | 5 | 0 | 0 | 0 |
| RPS12 | 0 | 0 | 0 | 2 | 3 | 3 | 6 | 4 | 3 | 3 | 4 | 5 |
| RPS13 | 2 | 4 | 2 | 3 | 3 | 5 | 2 | 1 | 3 | 0 | 0 | 0 |
| RPS14 | 1 | 1 | 0 | 8 | 7 | 6 | 6 | 6 | 5 | 3 | 3 | 4 |
| RPS15A | 1 | 1 | 1 | 7 | 9 | 7 | 2 | 2 | 3 | 0 | 2 | 0 |
| RPS16 | 3 | 6 | 6 | 12 | 14 | 12 | 12 | 10 | 10 | 0 | 0 | 0 |
| RPS17 | 2 | 2 | 3 | 8 | 5 | 5 | 5 | 4 | 3 | 0 | 2 | 0 |
| RPS18 | 5 | 5 | 7 | 9 | 11 | 12 | 12 | 12 | 12 | 0 | 0 | 0 |
| RPS19 | 0 | 1 | 0 | 2 | 1 | 2 | 2 | 1 | 1 | 0 | 0 | 0 |
| RPS2 | 5 | 3 | 2 | 19 | 22 | 12 | 14 | 10 | 14 | 0 | 0 | 0 |
| RPS20 | 1 | 1 | 0 | 4 | 2 | 4 | 2 | 2 | 2 | 0 | 0 | 0 |
| RPS23 | 3 | 2 | 1 | 8 | 8 | 8 | 8 | 7 | 7 | 0 | 0 | 0 |
| RPS24 | 0 | 0 | 0 | 2 | 1 | 1 | 0 | 0 | 0 | 0 | 0 | 0 |
| RPS25 | 2 | 0 | 1 | 1 | 1 | 1 | 3 | 3 | 2 | 0 | 0 | 0 |
| RPS26 | 1 | 1 | 1 | 1 | 2 | 0 | 1 | 1 | 2 | 0 | 0 | 0 |
| RPS27 | 1 | 0 | 0 | 1 | 2 | 3 | 4 | 3 | 2 | 0 | 0 | 0 |
| RPS27A | 66 | 111 | 104 | 18 | 24 | 22 | 23 | 24 | 17 | 0 | 0 | 0 |
| RPS3 | 11 | 8 | 10 | 40 | 40 | 33 | 34 | 28 | 25 | 6 | 7 | 8 |
| RPS3A | 5 | 3 | 0 | 19 | 16 | 18 | 7 | 8 | 13 | 0 | 0 | 0 |
| RPS4X | 7 | 7 | 3 | 22 | 23 | 17 | 13 | 9 | 9 | 0 | 0 | 0 |
| RPS5 | 3 | 3 | 0 | 11 | 9 | 4 | 9 | 7 | 6 | 4 | 0 | 0 |
| RPS6 | 0 | 0 | 0 | 4 | 3 | 2 | 2 | 2 | 0 | 2 | 0 | 2 |
| RPS7 | 2 | 2 | 1 | 5 | 9 | 5 | 3 | 4 | 6 | 0 | 0 | 0 |
| RPS8 | 2 | 1 | 0 | 9 | 10 | 9 | 3 | 2 | 2 | 0 | 0 | 0 |
| RPS9 | 2 | 1 | 2 | 7 | 5 | 7 | 4 | 5 | 4 | 0 | 0 | 2 |
| RPSA | 14 | 11 | 9 | 31 | 35 | 33 | 25 | 19 | 14 | 5 | 7 | 7 |
| RRBP1 | 0 | 0 | 0 | 1 | 0 | 0 | 4 | 2 | 3 | 7 | 5 | 3 |
| RRP9 | 0 | 0 | 0 | 10 | 7 | 5 | 9 | 5 | 8 | 4 | 7 | 5 |
| RTN4 | 8 | 6 | 3 | 15 | 26 | 17 | 19 | 21 | 20 | 0 | 0 | 0 |
| S100A16 | 1 | 3 | 2 | 0 | 0 | 0 | 0 | 0 | 0 | 0 | 0 | 0 |
| S100A9 | 1 | 13 | 0 | 0 | 0 | 3 | 0 | 0 | 0 | 0 | 0 | 0 |
| SARS | 5 | 4 | 2 | 14 | 15 | 12 | 19 | 12 | 17 | 2 | 3 | 5 |
| SART3 | 0 | 0 | 0 | 3 | 2 | 0 | 9 | 3 | 6 | 13 | 12 | 15 |
| SBDS | 0 | 1 | 0 | 3 | 2 | 1 | 5 | 2 | 3 | 0 | 0 | 0 |
| SCAMP2 | 2 | 3 | 2 | 1 | 3 | 3 | 0 | 0 | 0 | 0 | 0 | 0 |
| SCAMP3 | 2 | 4 | 2 | 4 | 6 | 4 | 1 | 3 | 1 | 0 | 0 | 0 |
| SEC23IP | 0 | 0 | 0 | 3 | 3 | 2 | 7 | 5 | 8 | 3 | 7 | 3 |
| SEC31A | 0 | 0 | 0 | 9 | 8 | 7 | 11 | 8 | 8 | 7 | 6 | 6 |
| SEPT11 | 3 | 1 | 0 | 6 | 8 | 5 | 10 | 9 | 9 | 18 | 15 | 11 |
| SEPT7 | 4 | 2 | 2 | 15 | 12 | 9 | 15 | 14 | 15 | 17 | 14 | 13 |
| SERPINH1 | 4 | 3 | 1 | 8 | 7 | 3 | 10 | 8 | 13 | 11 | 13 | 12 |
| SF3A1 | 0 | 0 | 0 | 1 | 1 | 1 | 5 | 4 | 7 | 5 | 9 | 9 |
| SF3A2 | 0 | 0 | 0 | 1 | 0 | 1 | 5 | 2 | 3 | 7 | 5 | 4 |
| SF3A3 | 0 | 0 | 0 | 6 | 6 | 4 | 6 | 12 | 9 | 10 | 9 | 10 |
| SF3B1 | 5 | 6 | 1 | 36 | 39 | 31 | 38 | 31 | 40 | 13 | 11 | 13 |
| SF3B2 | 0 | 0 | 0 | 0 | 1 | 1 | 3 | 5 | 3 | 2 | 2 | 3 |
| SF3B4 | 0 | 0 | 0 | 0 | 0 | 0 | 1 | 2 | 0 | 4 | 5 | 5 |
| SFPQ | 3 | 3 | 2 | 9 | 11 | 7 | 10 | 9 | 9 | 17 | 20 | 18 |
| SKIV2L2 | 1 | 2 | 0 | 9 | 15 | 14 | 18 | 15 | 16 | 16 | 11 | 16 |
| SLC25A5 | 5 | 0 | 0 | 0 | 0 | 0 | 0 | 0 | 0 | 0 | 0 | 0 |
| SLC3A2 | 25 | 30 | 33 | 9 | 11 | 9 | 5 | 6 | 4 | 2 | 2 | 3 |
| SMC1A | 1 | 0 | 0 | 24 | 17 | 17 | 52 | 42 | 46 | 23 | 22 | 17 |
| SMG1 | 0 | 0 | 0 | 3 | 3 | 0 | 3 | 0 | 2 | 0 | 0 | 0 |
| SND1 | 7 | 1 | 4 | 38 | 32 | 32 | 45 | 32 | 44 | 34 | 40 | 36 |
| SNRNP200 | 10 | 12 | 6 | 77 | 81 | 70 | 113 | 85 | 103 | 41 | 29 | 30 |
| SNRNP40 | 0 | 0 | 0 | 13 | 13 | 7 | 7 | 5 | 11 | 2 | 0 | 0 |
| SNRPA1 | 1 | 0 | 0 | 5 | 6 | 2 | 2 | 3 | 2 | 0 | 0 | 3 |
| SNRPB | 0 | 0 | 0 | 0 | 0 | 0 | 0 | 0 | 0 | 8 | 8 | 10 |
| SNRPD1 | 1 | 1 | 1 | 9 | 7 | 7 | 11 | 11 | 10 | 17 | 13 | 16 |
| SNRPD2 | 0 | 1 | 0 | 3 | 3 | 3 | 7 | 5 | 6 | 11 | 7 | 9 |
| SNRPD3 | 0 | 1 | 0 | 4 | 7 | 2 | 5 | 4 | 5 | 6 | 7 | 7 |
| SNRPF | 0 | 0 | 0 | 1 | 0 | 0 | 2 | 3 | 2 | 3 | 4 | 3 |
| SNRPG | 0 | 0 | 0 | 0 | 0 | 0 | 0 | 0 | 0 | 2 | 0 | 0 |
| SNTB1 | 12 | 12 | 11 | 1 | 1 | 0 | 0 | 0 | 0 | 0 | 0 | 0 |
| SNTB2 | 0 | 3 | 1 | 0 | 0 | 0 | 0 | 0 | 0 | 0 | 0 | 0 |
| SPAG9 | 0 | 0 | 0 | 0 | 0 | 0 | 0 | 0 | 0 | 0 | 2 | 0 |
| SPTAN1 | 26 | 10 | 12 | 47 | 48 | 44 | 166 | 136 | 165 | 257 | 242 | 255 |
| SPTBN1 | 23 | 15 | 9 | 62 | 70 | 57 | 167 | 144 | 168 | 216 | 208 | 194 |
| SRP14 | 0 | 1 | 0 | 2 | 2 | 2 | 2 | 2 | 2 | 0 | 0 | 0 |
| SRP68 | 0 | 0 | 0 | 3 | 2 | 0 | 2 | 0 | 1 | 0 | 0 | 0 |
| SRP72 | 0 | 0 | 0 | 3 | 4 | 1 | 4 | 3 | 3 | 0 | 2 | 2 |
| SRPK1 | 0 | 0 | 0 | 3 | 2 | 1 | 3 | 3 | 2 | 0 | 0 | 2 |
| SRRT | 0 | 2 | 0 | 10 | 8 | 7 | 11 | 4 | 6 | 3 | 4 | 5 |
| SRSF1 | 0 | 0 | 0 | 3 | 4 | 1 | 1 | 2 | 1 | 5 | 5 | 4 |
| SRSF2 | 0 | 0 | 0 | 0 | 0 | 0 | 0 | 0 | 0 | 3 | 3 | 2 |
| SRSF7 | 0 | 0 | 0 | 3 | 1 | 1 | 1 | 2 | 1 | 0 | 0 | 0 |
| SSB | 1 | 1 | 0 | 6 | 7 | 3 | 7 | 8 | 7 | 10 | 9 | 8 |
| SSBP1 | 0 | 0 | 0 | 0 | 0 | 0 | 4 | 2 | 2 | 9 | 10 | 10 |
| SSRP1 | 0 | 1 | 0 | 5 | 6 | 7 | 7 | 9 | 8 | 14 | 10 | 14 |
| STIP1 | 1 | 2 | 2 | 11 | 14 | 6 | 29 | 25 | 27 | 39 | 45 | 41 |
| STRAP | 4 | 4 | 3 | 16 | 19 | 17 | 22 | 18 | 18 | 9 | 10 | 15 |
| STRBP | 0 | 0 | 0 | 0 | 0 | 0 | 3 | 1 | 3 | 0 | 0 | 0 |
| SUB1 | 0 | 0 | 0 | 0 | 0 | 0 | 2 | 5 | 3 | 7 | 3 | 5 |
| SUCLG1 | 4 | 0 | 0 | 2 | 3 | 2 | 3 | 3 | 1 | 0 | 0 | 0 |
| SUPT16H | 1 | 1 | 0 | 9 | 6 | 7 | 11 | 11 | 10 | 13 | 10 | 15 |
| SUPT5H | 0 | 0 | 0 | 8 | 9 | 5 | 6 | 4 | 4 | 2 | 0 | 0 |
| SUPT6H | 0 | 0 | 0 | 1 | 1 | 0 | 5 | 4 | 3 | 11 | 9 | 5 |
| SYNCRIP | 3 | 2 | 0 | 6 | 5 | 3 | 7 | 6 | 4 | 17 | 17 | 18 |
| SYNE2 | 1 | 0 | 0 | 16 | 16 | 13 | 11 | 7 | 15 | 0 | 0 | 0 |
| TAGLN2 | 14 | 15 | 14 | 18 | 18 | 13 | 16 | 15 | 13 | 20 | 16 | 17 |
| TARDBP | 0 | 0 | 0 | 3 | 4 | 2 | 1 | 1 | 2 | 0 | 0 | 0 |
| TARS | 4 | 6 | 4 | 29 | 35 | 33 | 52 | 43 | 57 | 57 | 50 | 52 |
| TCP1 | 11 | 12 | 8 | 58 | 58 | 46 | 58 | 57 | 64 | 48 | 44 | 48 |
| TES | 0 | 0 | 0 | 0 | 0 | 0 | 4 | 1 | 5 | 7 | 5 | 7 |
| TFRC | 38 | 47 | 42 | 32 | 39 | 35 | 102 | 84 | 79 | 131 | 140 | 152 |
| THUMPD1 | 0 | 0 | 0 | 0 | 0 | 0 | 0 | 0 | 0 | 2 | 2 | 3 |
| TKT | 17 | 13 | 9 | 12 | 13 | 14 | 56 | 59 | 50 | 412 | 369 | 341 |
| TLN1 | 24 | 18 | 18 | 60 | 58 | 51 | 118 | 98 | 114 | 112 | 104 | 109 |
| TMED10 | 2 | 1 | 1 | 2 | 2 | 2 | 0 | 0 | 0 | 0 | 0 | 0 |
| TNPO1 | 3 | 1 | 2 | 8 | 11 | 9 | 16 | 11 | 17 | 14 | 12 | 12 |
| TOP1 | 0 | 0 | 0 | 1 | 4 | 3 | 8 | 15 | 6 | 5 | 4 | 4 |
| TOP2A | 0 | 0 | 0 | 8 | 11 | 7 | 13 | 9 | 11 | 0 | 0 | 0 |
| TPR | 0 | 0 | 0 | 1 | 1 | 0 | 4 | 4 | 4 | 16 | 14 | 16 |
| TPT1 | 0 | 0 | 0 | 0 | 0 | 0 | 0 | 0 | 0 | 5 | 6 | 6 |
| TRA2B | 0 | 0 | 0 | 0 | 0 | 0 | 0 | 0 | 0 | 0 | 2 | 0 |
| TRAP1 | 0 | 0 | 0 | 0 | 0 | 0 | 0 | 0 | 0 | 12 | 19 | 17 |
| TRIM25 | 1 | 2 | 0 | 9 | 11 | 9 | 6 | 5 | 6 | 4 | 4 | 4 |
| TRIM28 | 4 | 2 | 1 | 25 | 25 | 23 | 23 | 22 | 22 | 9 | 8 | 5 |
| TRMT1 | 1 | 1 | 1 | 6 | 6 | 7 | 7 | 6 | 5 | 0 | 0 | 0 |
| TRMT6 | 0 | 0 | 0 | 15 | 10 | 8 | 13 | 5 | 6 | 0 | 0 | 0 |
| TSNAX | 0 | 0 | 0 | 0 | 1 | 0 | 10 | 11 | 11 | 15 | 17 | 13 |
| TUFM | 0 | 0 | 0 | 6 | 4 | 5 | 4 | 2 | 1 | 0 | 0 | 0 |
| TWF2 | 0 | 0 | 0 | 0 | 0 | 0 | 0 | 0 | 0 | 2 | 0 | 0 |
| TXN | 5 | 5 | 4 | 3 | 3 | 3 | 3 | 3 | 4 | 4 | 3 | 3 |
| TXNDC5 | 0 | 0 | 0 | 1 | 3 | 1 | 2 | 2 | 1 | 13 | 15 | 15 |
| U2AF2 | 1 | 1 | 1 | 4 | 6 | 2 | 4 | 5 | 5 | 9 | 10 | 7 |
| U2SURP | 0 | 1 | 0 | 3 | 3 | 2 | 4 | 5 | 1 | 4 | 3 | 2 |
| UBA1 | 37 | 30 | 22 | 73 | 76 | 62 | 105 | 78 | 105 | 106 | 102 | 112 |
| UBE2L3 | 2 | 2 | 2 | 0 | 1 | 0 | 1 | 1 | 1 | 5 | 4 | 5 |
| UBE2M | 1 | 2 | 1 | 3 | 2 | 2 | 1 | 1 | 2 | 4 | 2 | 3 |
| UBE2N | 4 | 7 | 6 | 1 | 1 | 1 | 1 | 0 | 0 | 4 | 5 | 5 |
| UBE2O | 0 | 0 | 0 | 3 | 1 | 0 | 2 | 1 | 2 | 0 | 2 | 0 |
| UBQLN1 | 0 | 0 | 0 | 0 | 0 | 0 | 0 | 0 | 0 | 3 | 2 | 0 |
| UCHL5 | 0 | 0 | 0 | 1 | 1 | 0 | 3 | 1 | 2 | 4 | 4 | 3 |
| UPF1 | 0 | 1 | 1 | 16 | 17 | 9 | 26 | 23 | 24 | 27 | 23 | 23 |
| USO1 | 0 | 0 | 0 | 7 | 5 | 3 | 12 | 9 | 12 | 12 | 14 | 11 |
| USP10 | 0 | 0 | 0 | 5 | 6 | 2 | 5 | 5 | 6 | 2 | 2 | 0 |
| UTP15 | 0 | 0 | 0 | 5 | 2 | 1 | 0 | 0 | 0 | 0 | 0 | 0 |
| VCP | 67 | 72 | 58 | 96 | 117 | 95 | 366 | 344 | 383 | 97 | 84 | 83 |
| VIL1 | 22 | 19 | 18 | 41 | 33 | 29 | 70 | 55 | 63 | 76 | 81 | 84 |
| VIM | 1 | 2 | 0 | 0 | 0 | 0 | 1 | 0 | 0 | 0 | 0 | 0 |
| WDR43 | 0 | 3 | 2 | 1 | 1 | 0 | 0 | 1 | 1 | 2 | 0 | 0 |
| WDR75 | 0 | 0 | 0 | 2 | 2 | 0 | 1 | 1 | 1 | 0 | 0 | 0 |
| XPO5 | 1 | 1 | 1 | 12 | 10 | 7 | 18 | 12 | 17 | 6 | 9 | 9 |
| XRCC5 | 17 | 11 | 13 | 58 | 61 | 45 | 93 | 70 | 83 | 66 | 50 | 59 |
| XRCC6 | 18 | 13 | 11 | 49 | 49 | 46 | 72 | 63 | 64 | 64 | 55 | 54 |
| XRN2 | 0 | 0 | 0 | 6 | 6 | 5 | 5 | 3 | 4 | 22 | 18 | 19 |
| YARS | 0 | 0 | 0 | 1 | 2 | 2 | 2 | 3 | 1 | 2 | 4 | 5 |
| YBX1 | 0 | 0 | 0 | 3 | 3 | 0 | 10 | 11 | 5 | 9 | 8 | 9 |
| YWHAB | 11 | 16 | 15 | 10 | 13 | 12 | 21 | 20 | 20 | 47 | 56 | 56 |
| YWHAE | 16 | 23 | 16 | 12 | 12 | 11 | 24 | 23 | 24 | 82 | 84 | 84 |
| YWHAG | 7 | 14 | 8 | 10 | 15 | 10 | 22 | 22 | 24 | 47 | 48 | 56 |
| YWHAZ | 19 | 19 | 12 | 8 | 10 | 10 | 28 | 20 | 24 | 57 | 58 | 58 |
| ZC3H15 | 0 | 0 | 0 | 0 | 0 | 0 | 0 | 0 | 0 | 2 | 2 | 2 |
| ZC3HAV1 | 2 | 2 | 2 | 2 | 1 | 1 | 0 | 0 | 1 | 0 | 0 | 0 |
| ZFR | 0 | 0 | 0 | 2 | 1 | 1 | 4 | 1 | 2 | 6 | 5 | 5 |
| ZNF638 | 0 | 0 | 0 | 0 | 0 | 0 | 0 | 0 | 0 | 0 | 3 | 0 |

In summary, supermeres display a distinct signature of small exRNAs with very high expression of specific miRNAs, including miR-1246, and supermeres are enriched for the miRNA-binding proteins AGO1, AGO2, hnRNPA2B1 and XPO5. High levels of AGO2 secretion in exomeres and supermeres may be a common feature of cancer cells.

### Supermeres Affect In Vivo Levels of Liver Lipids, Glycogen, and Phosphorylated AKT and ERK1/2

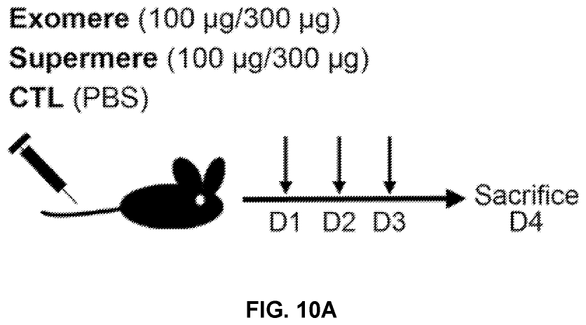

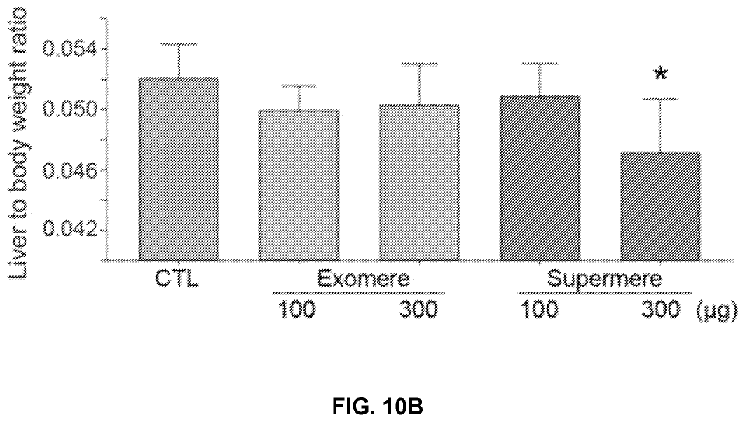

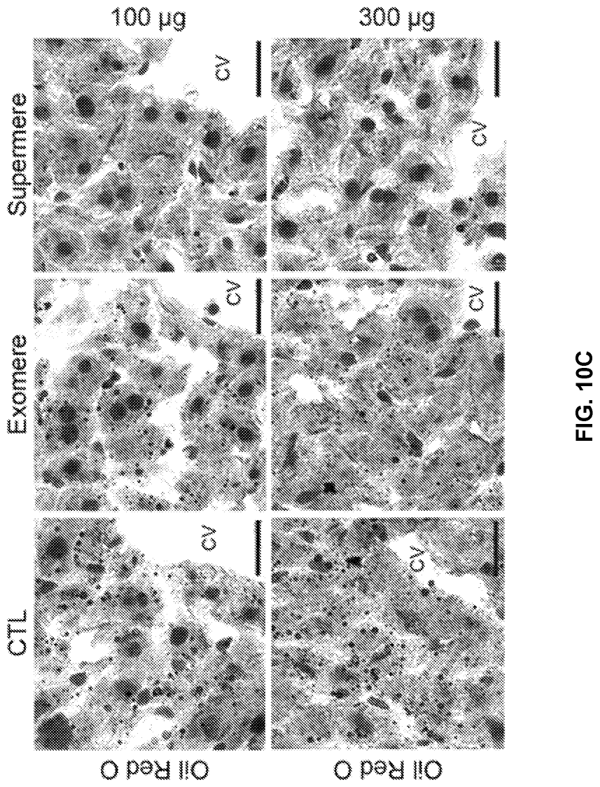

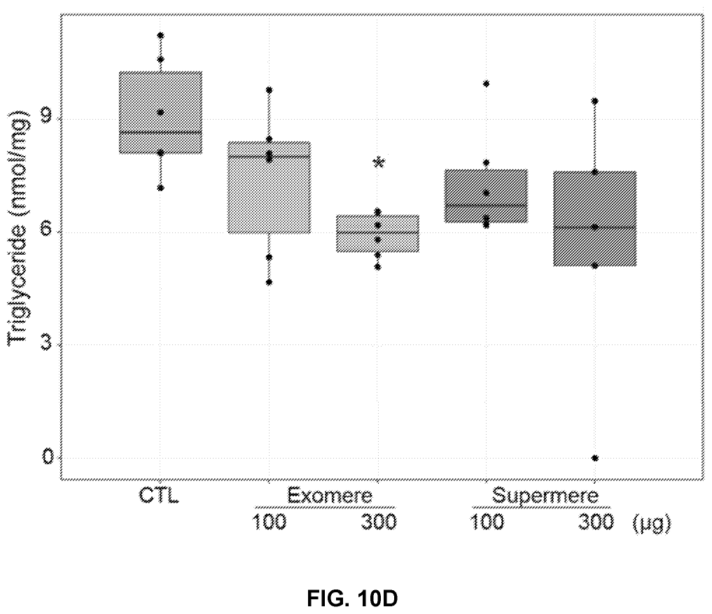

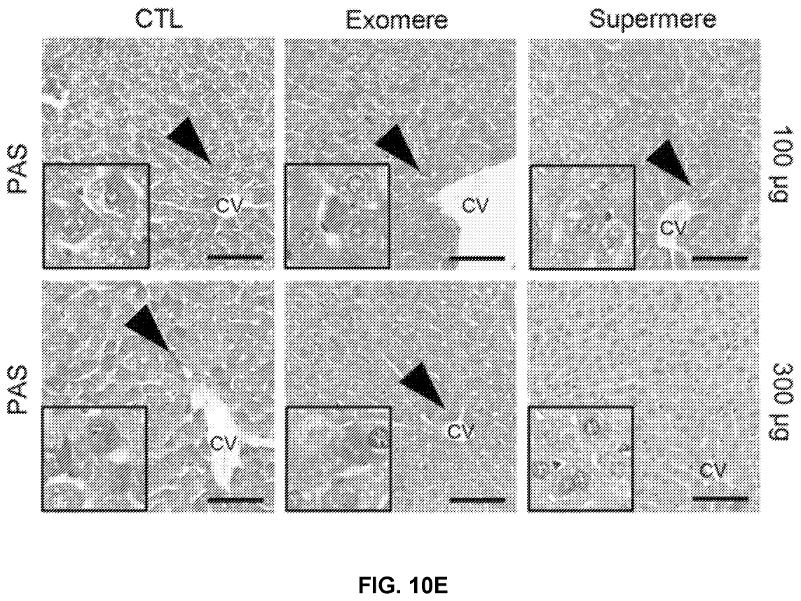

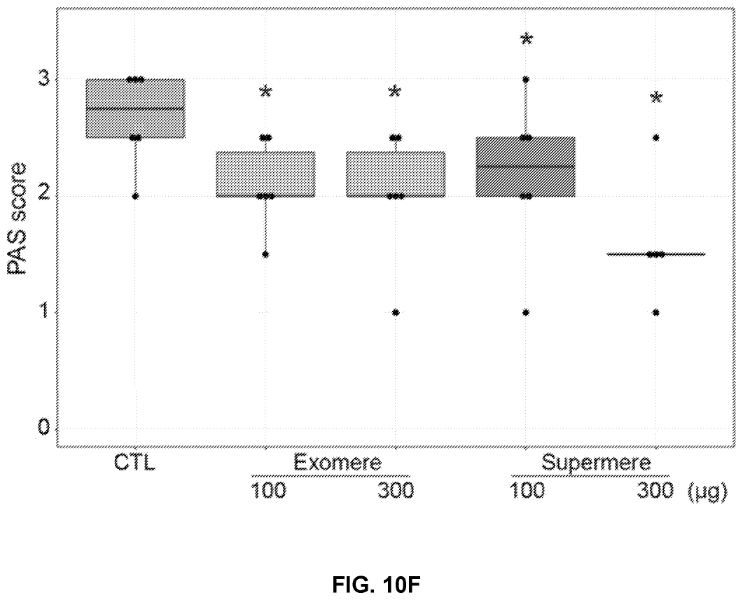

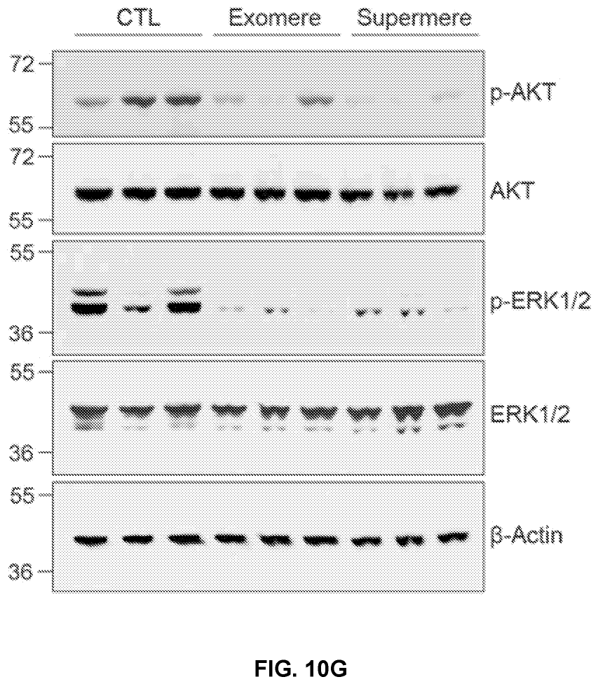

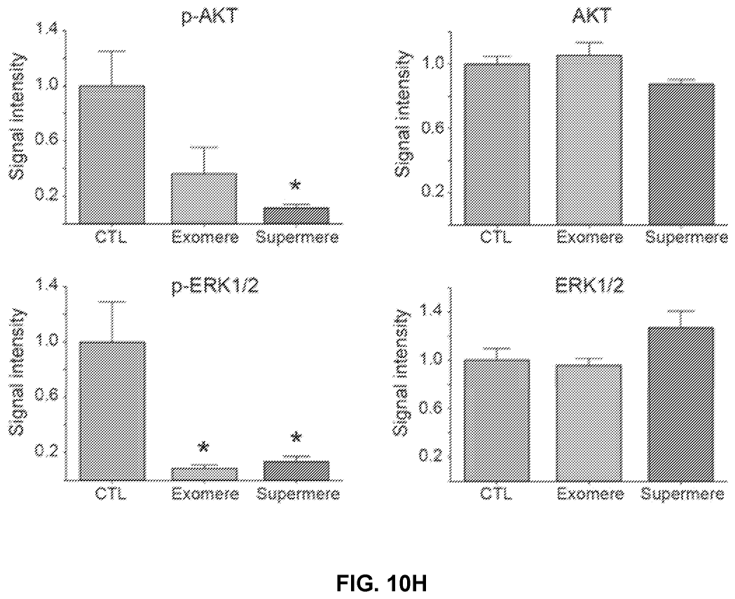

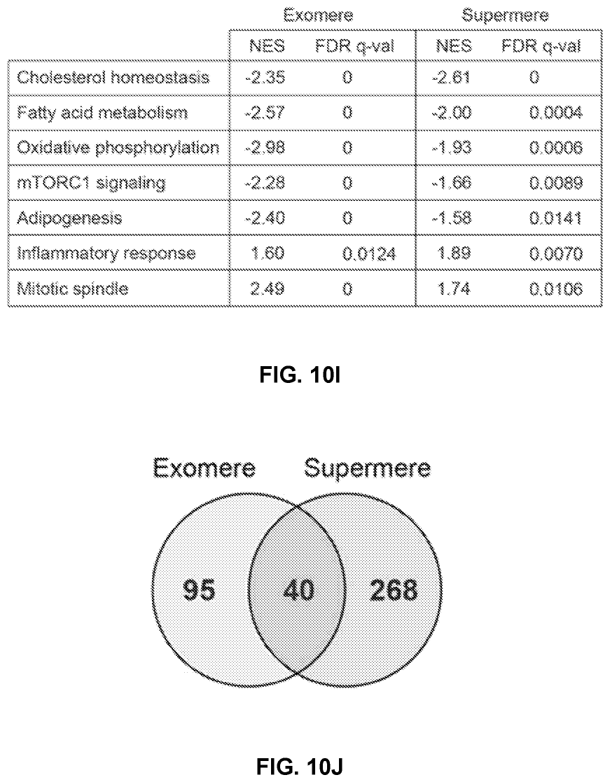

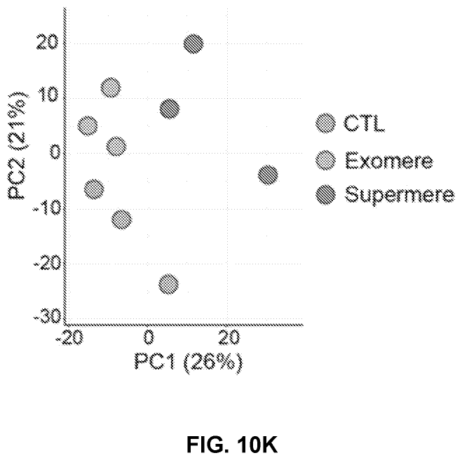

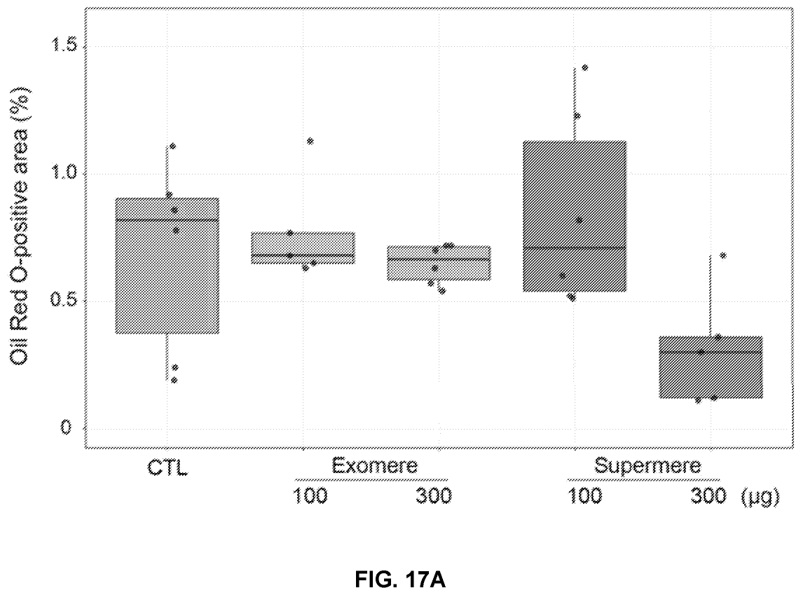

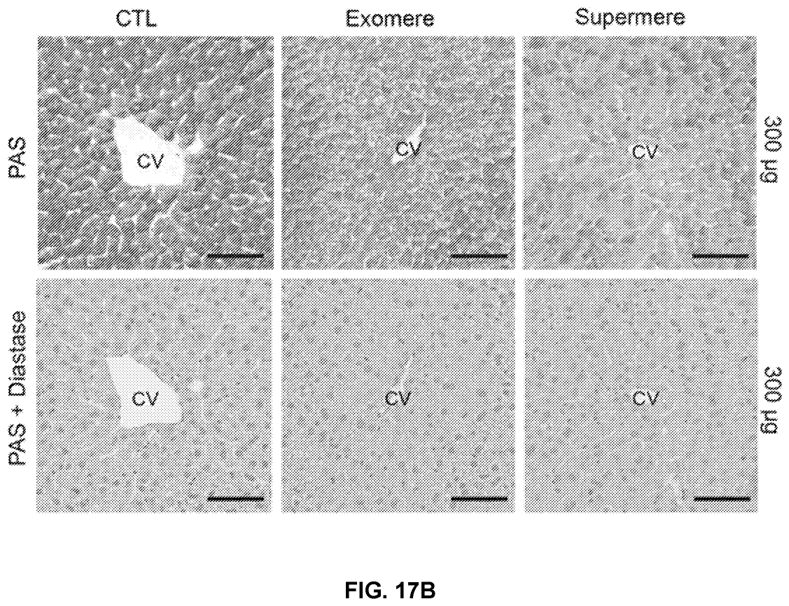

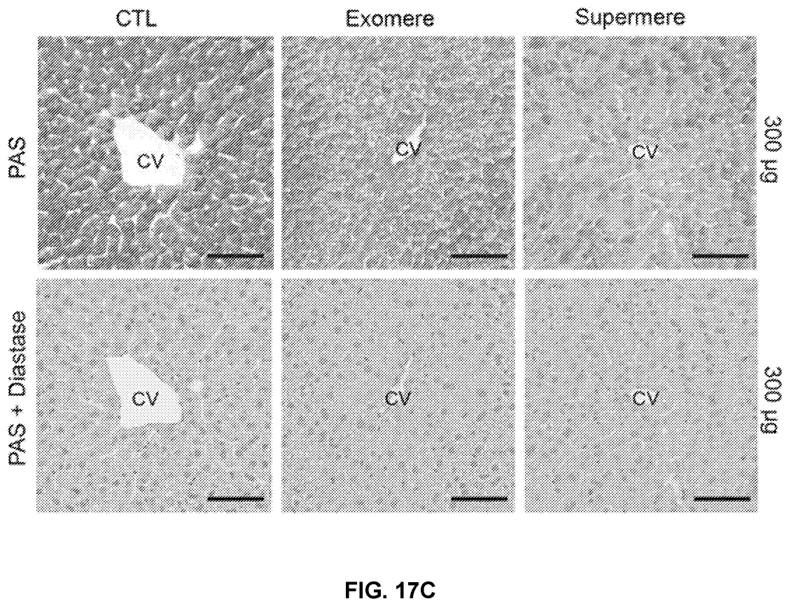

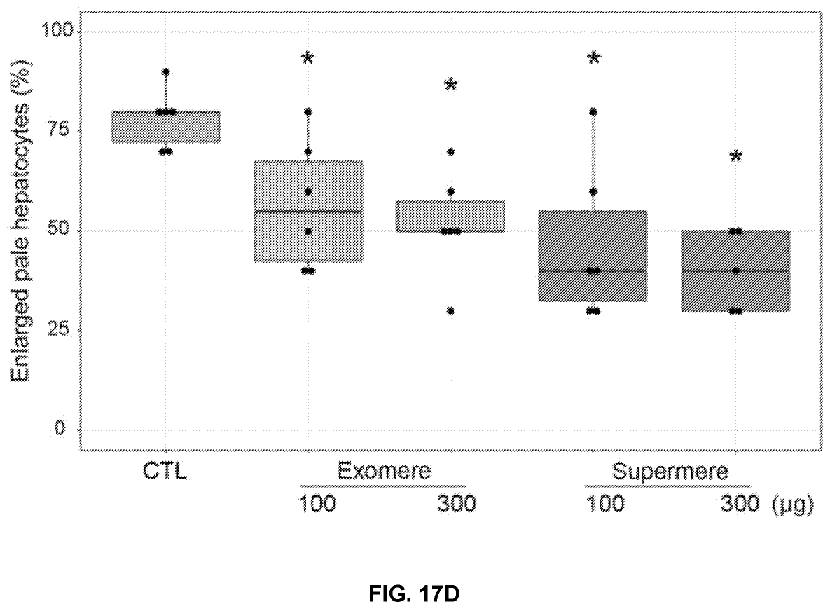

Since supermeres are enriched for proteins involved in metabolism (FIG. 7A-7E) and the liver is one of the main targets for supermere biodistribution (FIG. 5G), the acute effects on the liver following systemic delivery of supermeres was examined. Mice were injected with supermeres or exomeres via tail vein (FIG. 10A). No gross effects on the liver were observed, but there was a supemere-selective decrease in liver to body ratio at the higher concentrations of supermeres (FIG. 10B). There was a subsequent reduction in the number and size of hepatic lipid droplets following tail vein injection of mice with both supermeres and exomeres (FIG. 10C, 17A), as well as a trend towards lower triglyceride concentrations in liver tissue (FIG. 10D). Following supermere or exomere treatment, hepatocytes also displayed a significant reduction in glycogen levels (FIG. 10E, 10F, 17B). While control mice exhibited uniformly pale, large hepatocytes, the hepatocytes of treated mice were smaller, especially around centrilobular veins that comprise metabolic zone 3 that is particularly active in glycolysis and lipogenesis (Kietzmann, T. Redox Biol 2017 11:622-630). Blinded scoring confirmed a significant reduction of enlarged and pale hepatocytes in mice treated with supermeres and exomeres (FIG. 17C,17D). AKT and ERK1/2 signaling are known to regulate glucose and lipid metabolism (Hoxhaj, G. & Manning, B. D. Nat Rev Cancer 2020 20:74-88; Lavoie, H., et al. Nat Rev Mol Cell Biol 2020 21:607-632). In accordance with the in vivo observations, there was a significant reduction in phosphorylated (p)-AKT and p-ERK1/2 in liver cells following supermere treatment (FIG. 10G,10H). To discern differences in the effects of supermeres and exomeres, RNA-seq analysis of whole liver tissue was performed. GSEA analysis revealed that there were pathways significantly downregulated by both nanoparticles including cholesterol homeostasis, fatty acid metabolism, oxidative phosphorylation and adipogenesis that might, at least in part, be responsible for the effects observed in the liver (FIG. 10I and Table 6). Interestingly, both supermere- and exomere-treated mice also had a marked downregulation of hepatic mTORC1 signaling, a major nutrient sensitive regulator of growth (Umemura, A., et al. Cell Metab 2014 20:133-144). However, despite these overall similarities, there were significant differences in gene expression between the two groups (FIG. 10J,10K), suggesting selectivity of these effects. In summary, exomeres and supermeres have potent and distinct effects on hepatic glucose and lipid metabolism, likely by modulation of AKT and ERK1/2 signaling.

### DPEP1 and CD73 in Classical Exosomes and FASN in Exomeres are Potential CRC Biomarkers

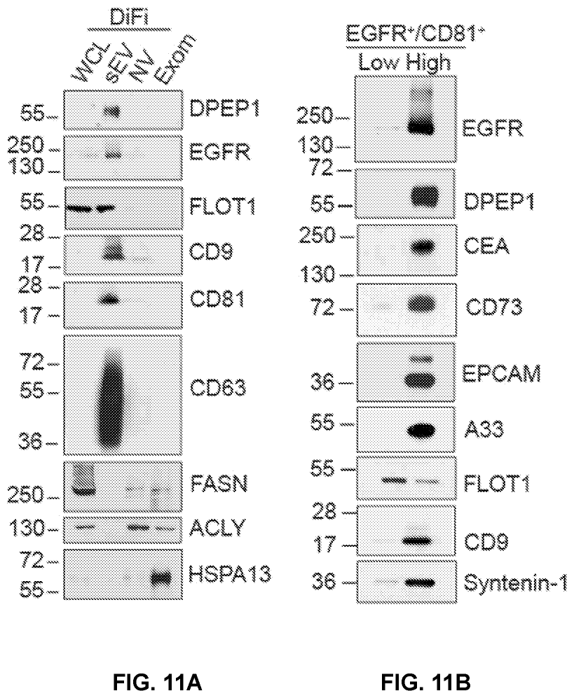

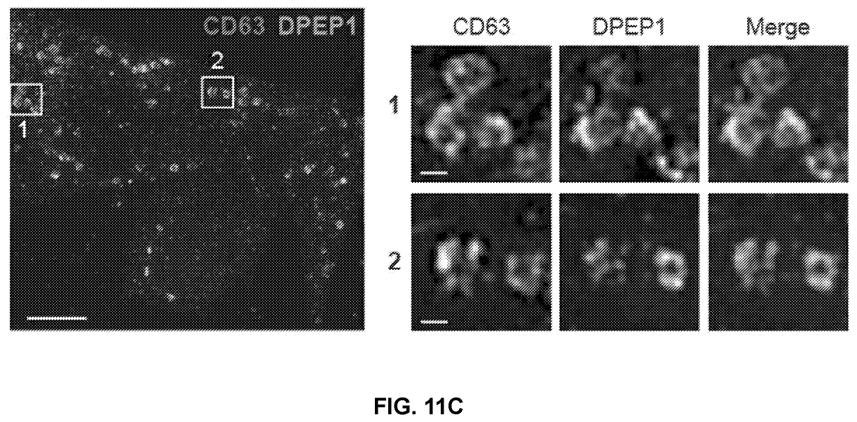

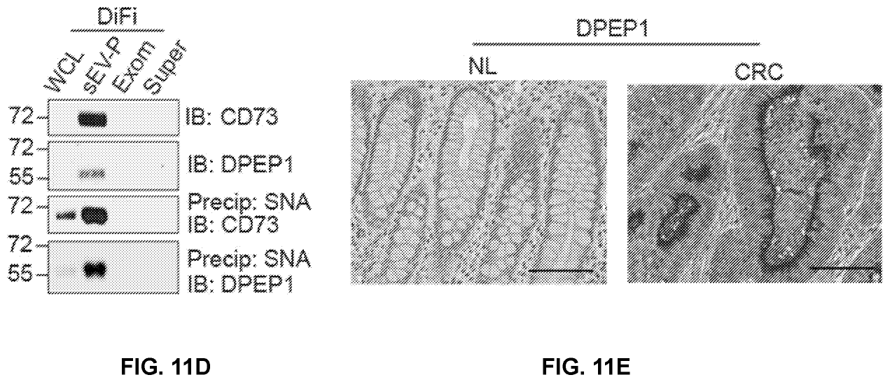

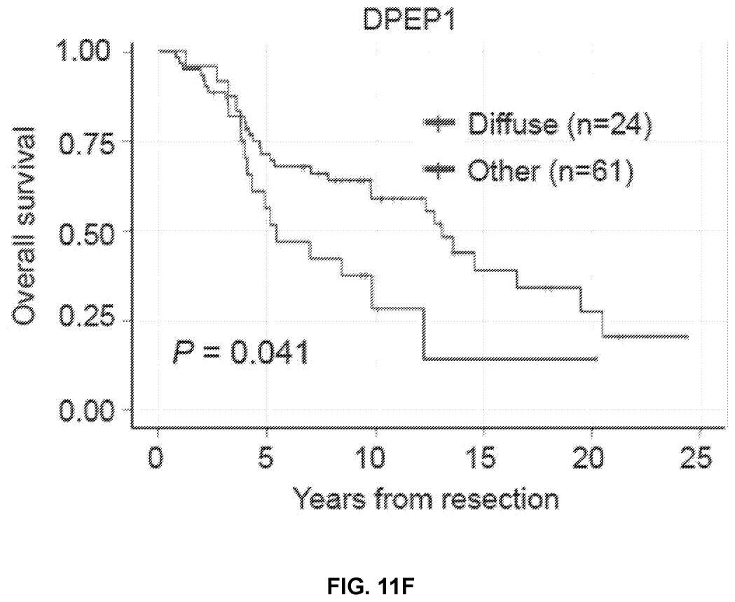

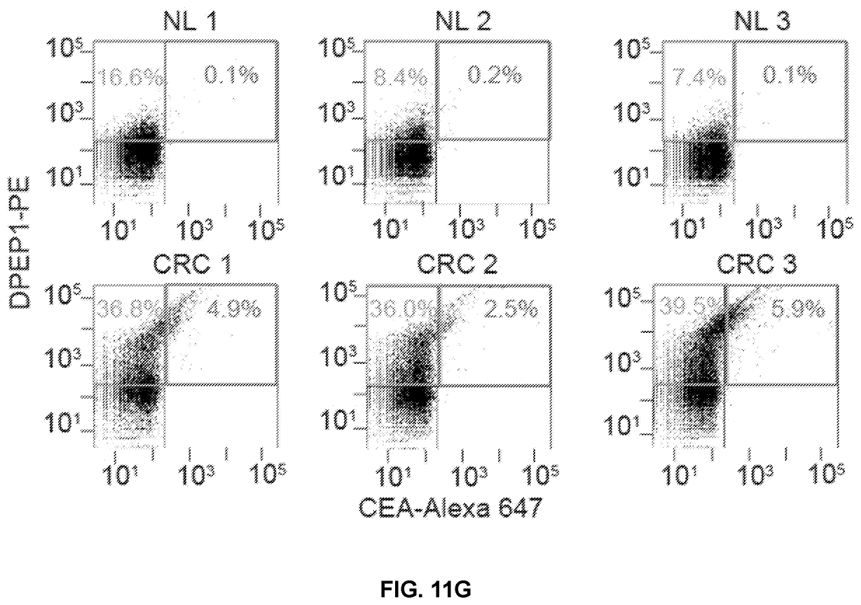

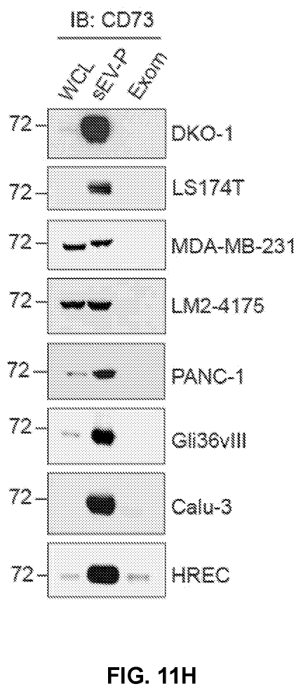

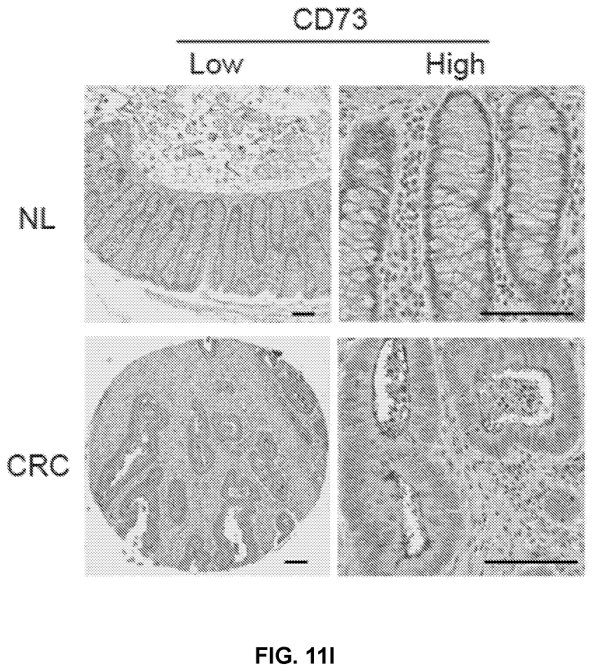

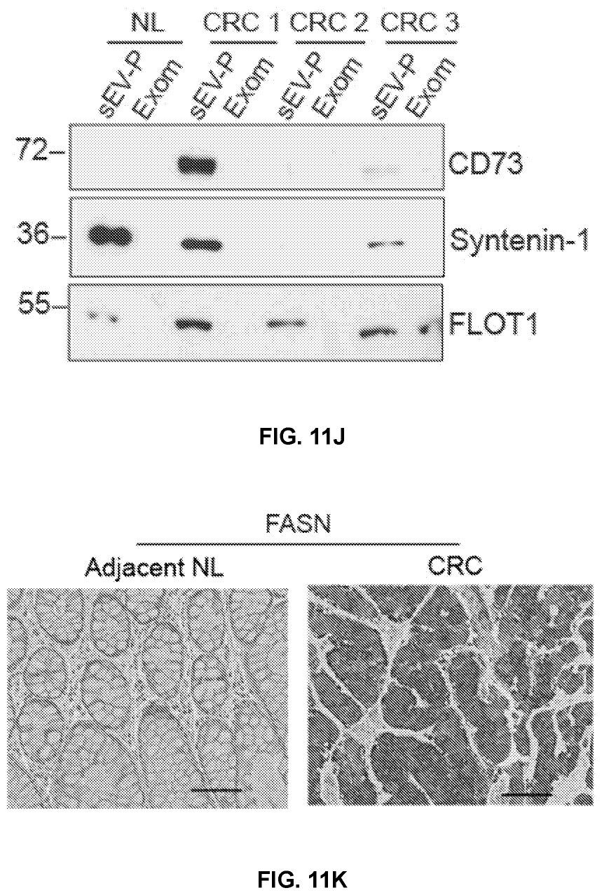

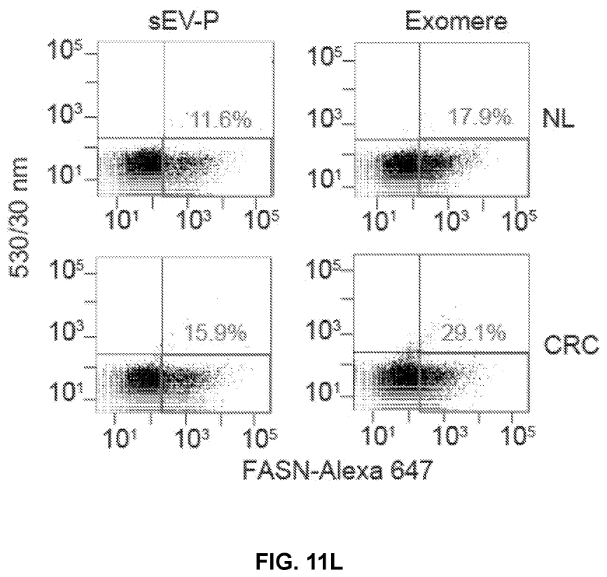

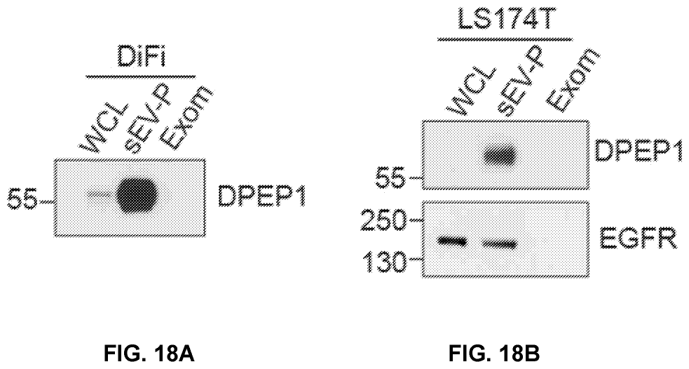

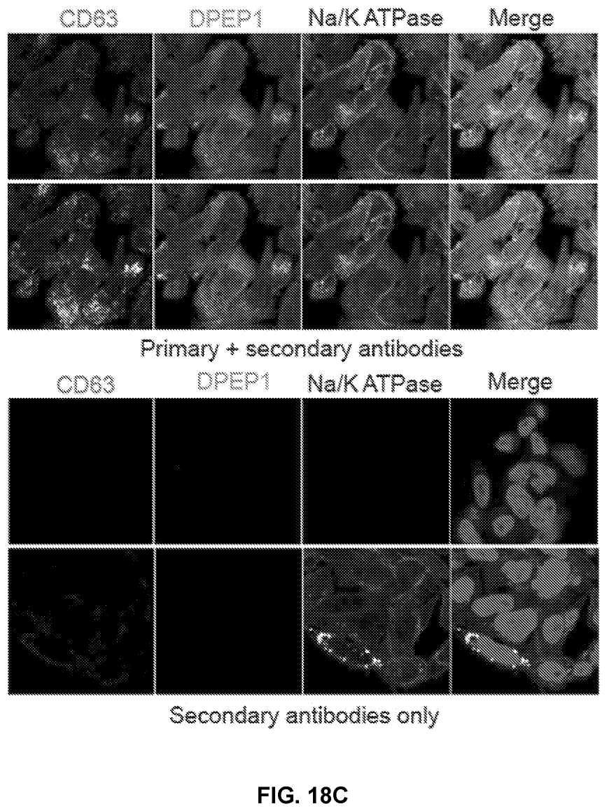

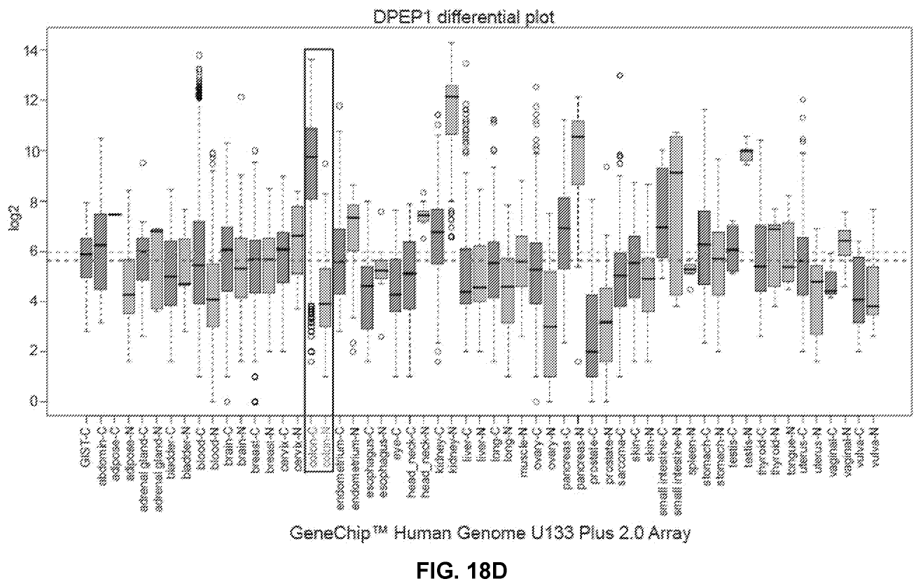

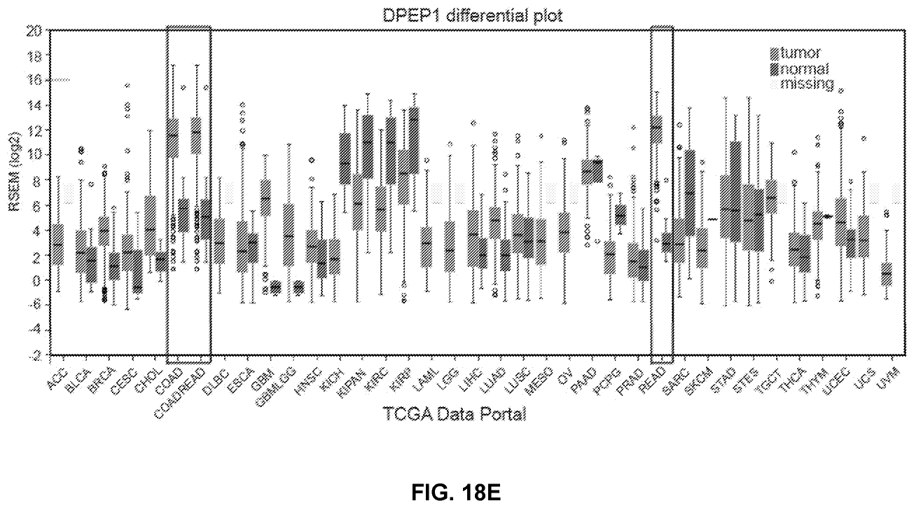

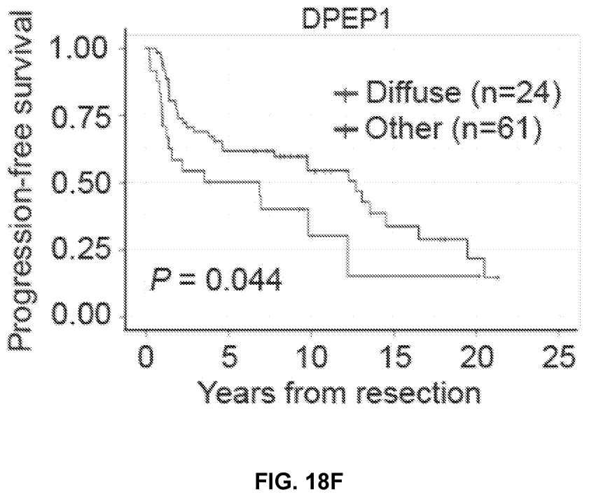

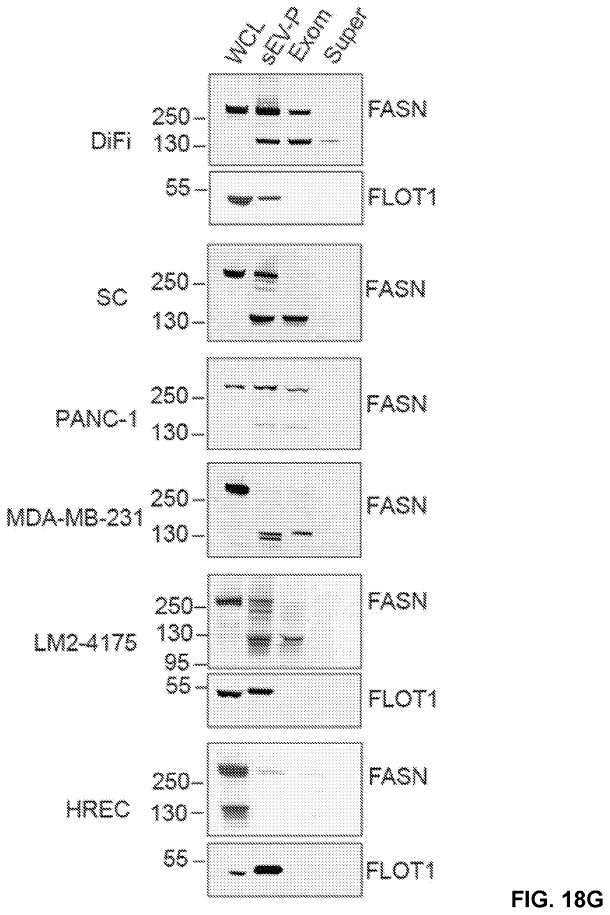

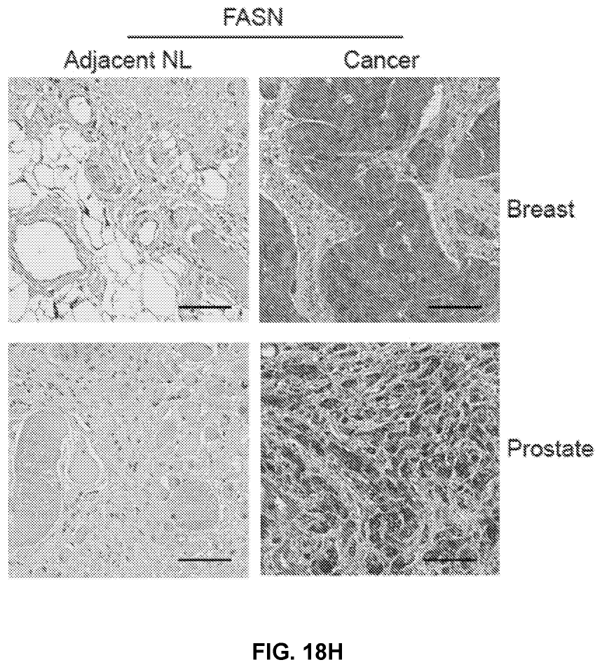

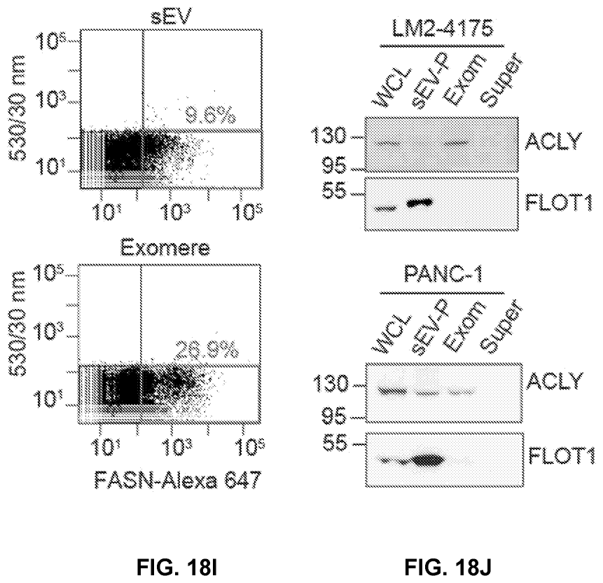

As the original intent of this project was to parse out the heterogeneity between sEVs and nanoparticles, the most abundant proteins in sEVs and exomeres of DiFi cells was next determined. DPEP1, a GPI-anchored zinc-dependent dipeptidase involved in glutathione metabolism, regulation of leukotriene activity (Nakagawa, H., et al. Cytogenet Cell Genet 1992 59:258-260) and neutrophil recruitment (Choudhury, S. R., et al. Cell 178:1205-1221 2019 e1217), as well as EGFR were the two proteins with highest spectral counts in gradient-purified DiFi sEVs (FIG. 11A, 18A). They were also present in the sEV-P derived from LS-174T cells (FIG. 18B), despite low expression in cell lysates. To determine if DPEP1 was present in classical exosomes (Jeppesen, D. K., et al. Cell 2019 177:428-445 e418), sEVs were sorted by using FAVS with fluorescently-labeled antibodies to EGFR and the exosomal marker, CD81 (FIG. 11B). Double-stained populations were analyzed and sorted into EGFR+/CD81+ high or low subpopulations (Zhang, Q., et al. Cell Rep 2019 27:940-954 e946). Notably, DPEP1, along with known CRC biomarkers (CEA, EPCAM and A33), were highly enriched in the EGFR⁺/CD81⁺ high population. CD73 (NT5E) was also highly enriched in this population (FIG. 11B). CD73 is a 5′-ecto-nucleotidase that catalyzes the conversion of extracellular ADP/AMP to adenosine, a known immunosuppressant. DPEP1, CEA and CD73 are all GPI-linked glycoproteins. Conversely, FLOT1 was more enriched in the EGFR⁺/CD81⁺ low populations, suggesting that FLOT1 is more associated with a different subset of exosomes and/or non-exosomal sEVs. These results underscore the heterogeneity of sEVs, and the utility of FAVS for analysis and sorting of distinct vesicle populations. DPEP1 co-localized with the canonical exosome marker CD63 in multivesicular endosomes (FIG. 11C,18C), further validating the presence of DPEP1 in classical exosomes. Furthermore, it was determined that DPEP1 and CD73 were α2,6-sialylated (FIG. 11D), likely due to ST6Gal-I (Zhang, Q., et al. Cell Rep 2019 27:940-954 e946).

Next, the clinical relevance of DPEP1 as a potential CRC biomarker was examined. Bioinformatic analysis of the U133Plus2 and TCGA Databases showed that DPEP1 is highly upregulated in CRC compared to normal colonic tissue (FIG. 18D,18E). Immunohistochemical analysis of clinically well-annotated TMAs of CRC revealed that DPEP1 staining was markedly increased in CRC but was undetectable in normal colonic mucosa (FIG. 11E). Cox regression analysis showed a significant inverse correlation between CRCs with diffuse cytoplasmic staining for DPEP1 and patient overall survival (FIG. 11F), as well as progression-free survival (FIG. 18F). By FAVS, it was demonstrated that sEVs double-positive for DPEP1 and CEA were much higher in plasma from CRC patients compared to normal controls, suggesting that DPEP1 may be a promising prognostic biomarker and therapeutic target for a subset of CRC patients (FIG. 11G).

DPEP1 has also been shown to be a receptor for neutrophil recruitment in various tissues. (Wang M. Nat Rev Nephrol. 2022 18(4): 199; Lau A, et al. Sci Adv. 2022 8 (5): eabm0142; Choudhury S R, et al. Cell. 2019 178(5): 1205-21 e17). DPEP1 on EVs could act as a decoy or a neutrophil recruitment factor in the tumor environment. Therefore, targeting DPEP1's ability to bind neutrophils as well as its enzymatic function could alter immune surveillance of tumors

Furthermore, sEVs derived from human cancer cell lines, DKO-1 and LS-174T (colon), MDA-MB-231 and its derivative LM2-4175 (breast), PANC-1 (pancreas), Gli36vIII (glioblastoma), Calu-3 (lung), as well as human normal renal epithelial cells (HREC), all had high levels of CD73 (FIG. 11H). This observation is supported by previous studies (Jeppesen, D. K., et al. Cell 2019 177:428-445 e418; Zhang, H., et al. Nat Cell Biol 2018 20:332-343; Kugeratski, F. G., et al. Nat Cell Biol 2021 23:631-641), and suggests that CD73 is a potential marker protein for sEVs. Immunohistochemical staining of CD73 in CRC tissues showed increased membranous and cytoplasmic CD73 immunoreactivity in the tumor compared to adjacent normal colonic mucosa (FIG. 11I); by immunoblotting, CD73 was detected in the sEV-P isolated from the plasma of two CRC patients but was not present in the third patient or in the normal individual (FIG. 11J).

The next goal was to examine proteins that are highly enriched in exomeres and the NV fraction. The most abundant proteins detected in DiFi-derived exomeres and the NV fraction were β-actin and fatty acid synthase (FASN) (FIG. 6C,11A). FASN was also expressed in exomeres and NV fractions released from other cell lines (FIG. 18). FASN is a multienzyme catalyzing the synthesis of 16-carbon fatty acid palmitate from acetyl-CoA and malonyl-CoA (Menendez, J. A. & Lupu, R. Nat Rev Cancer 2007 7:763-777). Strong immunohistochemical staining for FASN was observed in CRC but was absent in adjacent normal mucosa (FIG. 11K). FASN staining was also much higher in breast and prostate tumors compared to adjacent normal mucosa (FIG. 18H). To assess if FASN could be detected in exomeres, it was first shown that it is highly enriched in exomeres from DiFi cells by FAVS (FIG. 18I), and then, as proof-of-principle by using FAVs, higher levels of FASN were detected in exomeres isolated from the plasma of a CRC patient compared to a normal control (FIG. 11L). In addition to FASN, other enzymes related to lipogenesis were enriched in exomeres and the NV fraction, including ATP citrate lyase (ACLY), acetyl-coenzyme A synthetase (ACSS2), acetyl-CoA carboxylase 1 (ACACA) and isocitrate dehydrogenase 1 (IDH1). ACLY is a key metabolic enzyme that catalyzes the ATP-dependent conversion of citrate and coenzyme A to acetyl-CoA, which is a central metabolite for de novo fatty acid and cholesterol biosynthesis. High expression of ACLY was confirmed in the NV fraction and exomeres derived from DiFi, LM2-4175 and PANC-1 cells (FIG. 11A,18J).

In summary, DPEP1 and CD73 were identified in classical exosomes, as well as FASN in exomeres, to be potential CRC biomarkers and druggable targets. These results highlight the benefits of parsing distinct extracellular compartments to identify new biomolecules of clinical interest and to assign cargo to their correct carrier.

### Discussion

Heterogeneity of extracellular vesicles and nanoparticle populations is a major challenge in the EV field (Jeppesen, D. K., et al. Cell 2019 177:428-445 e418; Zhang, H., et al. Nat Cell Biol 2018 20:332-343; Zhang, Q., et al. Cell Rep 2019 27:940-954 e946; Mathieu, M., et al. Nat Cell Biol 2019 21:9-17; van Niel, G., et al. Nat Rev Mol Cell Biol 2018 19:213-228). This Example reports the isolation and characterization of a new extracellular nanoparticle that is termed the supermere. Supermeres are distinct from exomeres in terms of size, morphology, composition, cellular uptake dynamics, and tissue distribution. They contain many proteins previously reported to be associated with exosomes (van Niel, G., et al. Nat Rev Mol Cell Biol 2018 19:213-228). For example, TGFBI, the most abundant protein in supermeres, is purportedly a component of EVs from mesenchymal stromal cells (Ruiz, M., et al. Biomaterials 2020 226:119544). Based on the inverse correlation between high immunoreactivity for TGFBI in CRC and both overall and progression-free survival, as well as increased levels in supermeres isolated from the plasma of CRC patients compared to normal individuals, it is proposed that TGFBI levels may be a useful marker in liquid biopsies for CRC patients. Argonaute proteins, including AGO1 and AGO2, were presumed exosomal proteins, but refinements in purification demonstrate that these miRNA-binding proteins are predominantly non-vesicular (Jeppesen, D. K., et al. Cell 2019 177:428-445 e418; Temoche-Diaz, M. M., et al. Elife 2019 8), and it was shown that AGO1 and AGO2 are highly associated with supermeres. Other known RNA-binding proteins were also highly enriched in supermeres, highlighting the emerging appreciation that a significant proportion of extracellular RNAs (exRNAs) and RNA-binding proteins are not associated with EVs (Jeppesen, D. K., et al. Cell 2019 177:428-445 e418; Tosar, J. P., et al. Trends Biochem Sci 2021). Many miRNAs barely detectable or undetectable at the cellular level are highly and selectively enriched in supermeres. For example, miR-1246, which has been linked to serum exosomes in CRC patients (Cooks, T., et al. Nat Commun 2018 9:771), is the most highly expressed and highly enriched miRNA in supermeres. The strong staining of miR-1246 in CRC tissue compared to normal colonic mucosa supports a potential role for miR-1246 in the pathogenesis of CRC. Supermeres and exomeres are not the only non-vesicular extracellular nanoparticles capable of transporting miRNA. In particular, plasma and serum contain large amounts of 7-14 nm HDL particles, known to contain miRNA (Li, K., et al. Methods Mol Biol 2018 1740:139-153; Michell, D. L., et al. J Vis Exp 2016). All the cell line-derived supermere samples generated for this work were from serum-free conditions, and ApoA1 or ApoA2 (the most abundant proteins of HDL complexes) could not be detected in any of the samples by proteomic analysis. However, efficient purification from HDL-rich blood may benefit from additional approaches, perhaps utilizing a combination of high-resolution density gradient fractionation (Jeppesen, D. K., et al. Cell 2019 177:428-445 e418) and FPLC or size-exclusion chromatography (Li, K., et al. Methods Mol Biol 2018 1740:139-153; Michell, D. L., et al. J Vis Exp 2016), for improved separation of sEVs, exomeres, supermeres and HDL particles.

As disclosed herein, supermeres and exomeres isolated from cetuximab-resistant SC and CC-CR cells can transfer cetuximab resistance to cetuximab-sensitive cells. Activation of the receptor tyrosine kinases MET and RON induce de novo cetuximab resistance in SC cells (Li, C., et al. Proc Natl Acad Sci USA 2017 114:E2852-E2861). In CC-CR cells, upregulation of a long non-coding RNA (lncRNA), MIR100HG, and two embedded miRNAs, miR-100 and miR-125b, is responsible for this acquired mode of cetuximab resistance (Lu, Y., et al. Nat Med 2017 23:1331-1341). Thus, multiple cargos, including proteins and RNA (mRNA, miRNA, and lncRNA) carried by nanoparticles may contribute to these modes of drug resistance. The identity of these cargos, and whether they act independently or cooperatively in cetuximab resistance, await further investigation.

The Warburg effect, a cancer cell preference for glycolysis in the presence of oxygen, features enhanced lactate secretion that contributes to acidification of the tumor microenvironment and extracellular matrix degradation (Brooks, G. A. Cell Metab 2018 27:757-785). Increased lactate secretion also has been linked to resistance to drugs targeting EGFR and MET (Apicella, M., et al. Cell Metab 2018 28:848-865 e846). Cancer cell-derived supermeres contain large amounts of glycolytic enzymes and their addition to recipient cells results in increased lactate secretion. Furthermore, mice treated with supermeres resulted in reduced levels of lipids and glycogen in the liver. There was a supermere-selective striking decrease in liver to body weight ratio, a remarkable finding as the liver to body weight ratio is usually highly conserved. Nevertheless, there were common pathways downregulated and upregulated in the liver by both supermeres and exomeres. Notably, there was common downregulation of the mechanistic target of rapamycin complex 1 (mTORC1) pathway, a major nutrient-sensitive regulator of growth (Umemura, A., et al. Cell Metab 2014 20:133-144). The observe liver phenotype is similar to that reported with hepatic mTORC1 inhibition in which there was decreased hepatic steatosis and an increased inflammatory response (Umemura, A., et al. Cell Metab 2014 20:133-144).

Shedding or release of membrane receptors to the extracellular environment is associated with a number of disease states (Lichtenthaler, S. F., et al. EMBO J 2018 37) and drug resistance (Miller, M. A., et al. Clin Cancer Res 2017 23:623-629). Secretion of full-length transmembrane receptors is a distinctive feature of sEVs/exosomes but the ectodomain of many clinically relevant transmembrane receptors, including MET, GPC1, CEA, ACE, ACE2 and APP, are highly abundant in supermeres. As an important example, the secreted receptor ACE2 in sEVs and/or extracellular nanoparticles may act as a decoy for SARS-CoV-2 to attenuate infection, as has been demonstrated for human soluble recombinant ACE2 (Zhang, Q., et al. Gastroenterology 2021 160:958-961 e953; Monteil, V., et al. Cell 2020 181:905-913 e907). GPI attached to the C-terminus of a protein enables it to be anchored to the membrane of cells or EVs, and many GPI-anchored proteins of clinical importance, including GPC1, CEA, DPEP1 and CD73, have been detected in the extracellular space and ascribed to exosomes. However, as one example, upon closer inspection, GPC1 is less associated with exosomes, or other sEVs, but rather is enriched in exomeres and supermeres. The apparent molecular weight of a detached GPI-anchored protein will not differ noticeably from a membrane-embedded GPI-anchored protein, hence some of these proteins have been presumed to be associated with exosomes or other EVs. Yet other GPI-anchored proteins, e.g., DPEP1 and CD73, are strongly associated with EGFR/CD81-positive exosomes and do not appear to be liberated from their GPI anchor to any significant degree. DPEP1 was recently identified as an adhesion receptor on liver and lung endothelial cells for neutrophil recruitment, and targeting DPEP1 reduced mortality in murine models of sepsis (Choudhury, S. R., et al. Cell 178:1205-1221 2019 e1217). As disclosed herein, increased diffuse DPEP1 staining is associated with overall and progression-free survival in CRC and that there are significantly increased levels of DPEP1/CEA-positive exosomes in the plasma of CRC patients. High levels of CD73 have been linked to immune suppression and tumor progression due to the generation of extracellular adenosine (Antonioli, L., et al. Trends Cancer 2016 2:95-109). CD73 is upregulated in many cancers, including CRC (Hammami, A., et al. Semin Immunol 2019 42:101304; Yu, M., et al. Nat Commun 2020 11:515); there was increased CD73 in CRC tumor tissue as well as demonstrate that CD73-positive exosomes can be detected in CRC plasma.

Based on these findings, it is proposed that TGFBI, ENO1 and GPC1 may be useful markers for extracellular nanoparticles (exomeres and supermeres), while HSPA13 and ENO2 are more specifically associated with supermeres. Full-length CD73 may be a useful general marker for sEVs. Supermeres contain a number of clinically relevant biomolecules, such as PCSK9, ACE, ACE2, MET, GPC1 and APP, and supermeres are efficiently taken up in many organs compared to sEVs and exomeres, including the heart and brain. Some fruitful areas of future investigation will be determining the biogeneis of supermeres and exomeres and exploring the physiological and pathophysiological consequences of these nanoparticles and their cargo.

In summary, supermeres are a new type of circulating extracellular nanoparticle. Supermeres are enriched in proteins central to a number of disease states, including cancer, COVID-19, cardiovascular disease and Alzheimer's disease. Many of these proteins have previously ascribed to exosomes or other EVs. These findings serve to highlight the importance of parsing the exact extracellular compartment that contains a biomolecule of interest. Supermeres are also functional agents of intercellular communication that are efficiently taken up by multiple organs, including liver, lung, colon, heart and brain. Supermeres thus takes their place alongside exosomes, EVs and exomeres as attractive targets for liquid biopsies and potential therapeutic drug targets.

### Methods

Cell lines. LS174T, PANC-1, Calu-3, and Hela cell lines were from the American Type Culture Collection (ATCC), Human primary renal proximal tubule epithelial cells (HREC) are from Innovative BioTherapies. HCA-7, its derivatives SC, CC and CC-CR, DiFi, and LIM1215 were maintained in the Coffey lab. DKO-1 cells were obtained from Dr. T. Sasazuki at Kyushu University, Gli36 cells were obtained from Dr. X. Breakefield at Harvard Medical School, and MDA-MB-231 and LM2-4175 cells were obtained from Dr. J. Massagué at Memorial Sloan-Kettering Cancer Center.

Cell culture. Human CRC cell lines, DiFi, DKO-1, HCA-7-derived SC (Li, C., et al. Proc Natl Acad Sci USA 2017 114:E2852-E2861), CC, CC-CR, LS174T, LIM1215, human breast cell lines MDA-MB-231 and LM2-4175, pancreatic cancer cell line PANC1, and lung cancer cell line Calu-3, human glioblastoma cell line Gli36vIII, and Hela cells were cultured in Dulbecco's Modified Eagle's Medium (DMEM) supplemented with 10% bovine growth serum, 1% glutamine, 1% non-essential amino acids, and 1% penicillin/streptomycin at 37° C. in a 5% CO₂ humidified incubator. Cells were maintained by passage every 3-4 days at 70%-80% confluence and were routinely tested for mycoplasma contamination (Universal Mycoplasma Detection Kit, ATCC, Manassas, VA, USA). All cell culture media was purchased from Corning Cellgro (Manassas, VA), and all cell culture supplements were from Hyclone (Logan, UT) unless stated otherwise. Primary cultures of human renal proximal tubule epithelial cells (HREC) were generated from transplant discards purchased from Innovative BioTherapies (Ann Arbor, MI, USA). Primary cultures for production of extracellular vesicles were initiated at passage 2 as previously described (Jeppesen, D. K., et al. Cell 2019 177:428-445 e418), and cells were maintained in DMEM supplemented with 2 μg/ml Normocin, Insulin-Transferrin-Selenium (ITS), epidermal growth factor (EGF), hydrocortisone and T3 thyroid hormone. For 3D cultures, cells were cultured in type-1 collagen as previously described (Li, C., et al. Proc Natl Acad Sci USA 2017 114:E2852-E2861). Briefly, type-1 collagen was diluted at 2 mg/ml in DMEM containing 10% (vol/vol) FBS. Assays were set up using three collagen layers, 400 μl each, in 12-well culture dishes, with the middle layer containing the single-cell suspension at 5,000 cells/mL. Medium (400 μl) with or without reagents was added on top and changed every 2-3 days. Colonies were observed and counted after 14-17 days.

Extracellular vesicle and nanoparticle isolation from cultured cells grown in dishes. Extracellular nanoparticles were isolated from cell-conditioned medium as previously described (Zhang, Q., et al. Cell Rep 2019 27:940-954 e946), with minor modifications. Colon, breast, lung and pancreatic cells mentioned above were cultured in specific medium as described above until 80% confluent. The cells were then washed three times with PBS and cultured for 48 h in serum-free medium. For primary human kidney epithelial cells, cell-conditioned medium was collected from approximately 95% confluent cells grown for 96 h in cell culture flasks with DMEM without FBS. The serum-free conditioned medium was removed and centrifuged for 15 min at 1000×g to remove cellular debris, and the resulting supernatant was then filtered through a 0.22-μm polyethersulfone filter (Nalgene, Rochester, NY) to reduce microparticle contamination. The filtrate was concentrated with a 100,000 molecular-weight cutoff centrifugal concentrator (Millipore). The concentrate then was subjected to high-speed centrifugation at 167,000×g for 4 h in a SW 32 Ti Rotor Swinging Bucket rotor (k factor of 204, Beckman Coulter, Fullerton, CA), and the resulting sEV-enriched pellet was resuspended in PBS containing 25 mM HEPES (pH 7.2) and washed by centrifuging again at 167,000×g for 4 h. The washed pellet was designated as the sEV-P. To isolate exomeres, the supernatant collected from the 4 h ultracentrifugation was ultracentrifuged at 167,000×g for 16 h. The resulting pellet was resuspended in PBS containing 25 mM HEPES (pH 7.2) and washed by centrifuging again at 167,000×g for 16 h. The washed pellet was designated as exomeres. To isolate supermeres, the supernatant from the pelleting of exomeres was subjected to ultracentrifugation at 367,000×g (rₘₐₓ, 55,000 RPM) using a Beckman Coulter SW55 Ti rotor (k factor of 48, Beckman Coulter, Fullerton, CA) for 16 h. The resulting pellet was resuspended in PBS containing 25 mM HEPES (pH 7.2) and was designated supermeres. The protein concentrations of the nanoparticles were determined with Direct Detect™ (Millipore, Burlington, MA). At no time during the process were samples subjected to temperatures below 4° C.

Extracellular vesicle and nanoparticle isolation from cultured cells grown in bioreactors. DKO-1 cells were maintained in CELLine Adhere 1000 (CLAD1000) bioreactors (INTEGRA Biosciences AG, Zizers, Switzerland) at 37° C. in a 5% CO₂ humidified incubator, as previously described (Jeppesen, D. K., et al. Cell 2019 177:428-445 e418). Cell-conditioned medium was harvested from bioreactors every 48 h, starting from one week after inoculation of the bioreactor and continuing for a period of 4 weeks. Pellets of sEVs were generated as previously described (Jeppesen, D. K., et al. Cell 2019 177:428-445 e418). Exomeres and supermeres were isolated as described above. At no time during the process were samples subjected to temperatures below 4° C.

Extracellular vesicle and nanoparticle isolation from human plasma samples. All procedures on human peripheral blood specimens were approved and performed in accordance with the Vanderbilt University Medical Center Institutional Review Board (IRB #161529 and #151721). All human participants provided informed consent (clinical trial registration number: NCT03263429). Blood was drawn into BD Vacutainer Blood Collection Tubes (BD Bioscience) containing buffered sodium citrate as anticoagulants. The first tube drawn was discarded. Further processing of samples was initiated within 2 h of blood draw. Plasma was generated by centrifugation of the blood at 1,500×g for 15 min and then a second round of centrifugation of the supernatant at 3,000×g for 15 min to ensure that no platelets remained. The resulting plasma samples were diluted immediately approximately 1:20 in ice cold PBS and spun at 20,000×g for 30 min to pellet and remove large EVs and microparticles. Clarified supernatants were subjected to ultracentrifugation at 167,000×g for 4 h in a SW 32 Ti Swinging Bucket rotor (k factor of 204, Beckman Coulter, Fullerton, CA) to sediment the sEV pellet (sEV-P). Pellets of crude sEVs were resuspended in ice-cold PBS, tubes were filled with PBS-H (25 mM HEPES), and then subjected to ultracentrifugation at 167,000×g for 4 h. The washed pellet was resuspended in ice-cold PBS-H. Pellets of exomeres and supermeres were generated as described above. The protein concentrations of the nanoparticles were determined with Direct Detect™ (Millipore, Burlington, MA). At no time during the process were plasma or plasma sEVs subjected to temperatures below 4° C.

High-resolution (12-36%) iodixanol density gradient fractionation. The gradient fractionation was performed as previously described (Jeppesen, D. K., et al. Cell 2019 177:428-445 e418). Twelve individual fractions of 1 ml were collected from the top of the gradient. Fractions 4 and 5, and fractions 8 and 9 were separately pooled. These two pools were then diluted 12-fold in PBS and subjected to ultracentrifugation at 120,000×g for 4 h at 4° C. using a SW41 TI Swinging Bucket rotor. The resulting pellets were lysed in cell lysis buffer for further proteomic and immunoblotting analysis.

Atomic Force Microscopy (AFM) imaging and analysis. Twenty microliters of isolated sEVs, the non-vesicular fraction (NV), exomeres and supermeres were diluted 1:1 with PBS and then incubated over (3-Aminopropyl) triethoxysilane (AP)-modified mica substrates (Ted Pella Inc, CA) for 3 min. To remove unbound particles, substrates were washed twice with 50 μL PBS and imaged in PBS at room temperature. Measurements were conducted in PBS using a Dimension FastScan Microscope (Bruker Instruments, Santa Barbara, CA) in off-resonance tapping mode, with ScanAsyst Fluid+tips (Bruker, CA) with nominal radius ˜2 nm and experimentally determined spring constants of 0.7 N/m. AFM images were taken at 256 samples per line, at 0.75 Hz. Images were exported offline and processed using Gwyddion or custom R software.

For statistical analysis, data were expressed as mean values±standard deviations. Statistical significance was identified by the Student's t-test for the differences among different samples. P values of less than 0.01 was considered to be statistically significant.

Negative stain transmission electron microscopy. Highly purified sEV fractions, NV fractions, exomeres and supermeres were prepared for transmission electron microscopy (TEM) as previously described (Jeppesen, D. K., et al. Cell 2019 177:428-445 e418).

Proteomics. Gradient fractionated sEVs, NV, exomeres and supermeres derived from DiFi cells or from PANC-1 and MDA-MB-231 cells were lysed in RIPA buffer, and equal amounts of protein were run on a NuPAGE Bis-Tris gel. LC/MS/MS was performed as previously described (Zhang, Q., et al. Cell Rep 2019 27:940-954 e946).

Proteomic analysis. Proteins with average count ≥1 in each fraction were considered detectable. Spectral counts of proteins were normalized by the total spectral counts and were log 2-transformed. Principal component analysis was performed to assess the similarity between samples. Differential expression between sEVs, NV, exomeres and supermeres was identified using Limma. Proteins with a fold change of >2 and a false discovery rate (FDR)<0.05 were considered to be significantly differentially expressed. Gene set enrichment analysis (GSEA) was implemented against three reference gene sets from the Molecular Signatures database (MSigDB v6.1): (H) hallmark gene sets (50 gene sets); (C2) KEGG gene sets (186 gene sets), and (C5) all gene ontology (GO) gene sets (5,917 gene sets). Default parameters were used to identify significantly enriched gene sets (min size 15, max size 500, FDR <0.25).

Structured illumination microscopy (SIM). 3D SIM imaging and processing was performed on a Nikon N-SIM structured illumination platform equipped with an Andor DU-897 EMCCD camera and a SR Apo TIRF 100× (1.49 NA, WD 0.12) oil immersion objective. Samples were imaged in PBS at room temperature. For calibration, 100 nm fluorescent (360/430 nm, 505/515 nm, 560/580 nm and 660/680 nm) beads (TetraSpeck™ Microspheres, Thermo Fisher Scientific, Waltham, MA, USA) were fixed and imaged. Images were analyzed using ImageJ software (National Institutes of Health, Bethesda, MD, USA).

Immunofluorescence staining for SIM. DiFi cells were cultured on 35-mm culture dishes with a 1.5 coverslip and 14-mm glass diameter (P35G-0.170-14-C, MatTek Corporation, Ashland, MA, USA) to approximately 50% confluence. Cells were fixed with 4% paraformaldehyde in PBS at room temperature for 20 min and then extracted for 5 min with 1% Triton X-100 in 4% paraformaldehyde in PBS as previously described (Jeppesen, D. K., et al. Cell 2019 177:428-445 e418). Cells were washed three times in PBS and blocked in 10% bovine serum albumin (BSA) in PBS. Cells were incubated with primary antibodies diluted in 10% BSA overnight at 4° C., washed with PBS for three times. The secondary Alexa Fluor antibodies (anti-rabbit conjugated to Alexa Fluor 488, and anti-mouse conjugated to Alexa Fluor 568) were prepared in blocking buffer and centrifuged at 13,000 rpm for 10 min before incubation on cells for 1 h at room temperature. Primary antibodies used were anti-DPEP1 (Sigma, HPA012783, 1:50), anti-CD63 (BD, 556019, 1:50).

Immunofluorescence staining for confocal microscopy. DiFi cells (2×10⁵) were cultured on 6-well plates for 2 days. Cells were then washed with PBS three times and fixed with 4% paraformaldehyde (PFA) for 10 min at room temperature and permeabilized with 0.5% Triton X-100 for 5 min at room temperature. Fixed cells were blocked for 2 h in 5% BSA at 4° C. and subsequently incubated overnight at 4° C. with primary antibodies in 5% BSA in PBS (anti-DPEP1, Sigma, HPA012783, 1:100; anti-CD63, BD, 556019, 1:100; anti-Na/KATPase a, Cell Signaling Technology, 3010, 1:500) The cells were washed three times in PBS and then incubated overnight with secondary antibodies in 5% BSA in PBS (anti-rabbit conjugated to Alexa Fluor 488, Cy3, anti-rabbit conjugated to Alexa Fluor 647). Immunofluorescence was analysed using a Zeiss LSM 710 confocal microscope. Microscopy was performed within the Vanderbilt Cell Imaging Shared Resource (CISR). All micrographs were taken using a 63× oil immersion objective lens.

Protein isolation from cells and all isolated fractions. Proteins were isolated as previously described (Zhang, Q., et al. Cell Rep 2019 27:940-954 e946).

Immunoblot analysis. Lysed samples were prepared in 5× sample buffer, heated to 70° C. for 10 min, or boiled for 5 min before being loaded on gels. The samples (30 μg) were separated on 4-12% SDS-PAGE Bis-Tris gels (Life Technologies) under either reducing or non-reducing conditions, depending on the subsequent use of primary antibody, before being transferred to nitrocellulose membranes (GE Healthcare, Pittsburgh, PA). Membranes were blocked for 1 h in 5% non-fat dry milk, or 5% bovine serum albumin, depending on the primary antibody subsequently used. Membranes was incubated with primary antibodies overnight at 4° C. After incubation with HRP-coupled secondary antibodies for 1 h, immunoblots were developed using chemiluminescence (Western Lightning Plus-ECL, PerkinElmer, Waltham, MA). The primary antibodies used were the following: anti-EEF1A1 (ab157455), anti-A33 (ab108938), anti-EPCAM (ab32392), anti-AGO2 (ab186733), anti-Syntenin-1 (ab133267), anti-ACE2 (ab108252), anti-APP (ab32136), anti-GPC1 (ab199343), anti-CEACAM5/CEA (ab133633), anti-TPI1 (ab96696), anti-LDHB (ab85319), anti-GPI (ab66340), anti-HSPA8 (ab51052), anti-PCSK9 (ab181142), anti-VPS35 (ab157220) and anti-MVP (ab175239) are from Abcam. Anti-MET (8198), anti-CEACAM5/CEA (2383), anti-CD73 (13160), anti-FASN (3180), anti-ACLY (4332), anti-AGO1 (5053), anti-XPO5 (12565), anti-HNRNPA2B1 (9304), anti-Alix (2171), anti-ALDOA (8060), anti-ENO1 (3810), anti-ENO2 (8171), anti-HK1 (2024), anti-PKM1/2 (3190), anti-LDHA (3582), anti-pAKT (9271), anti-AKT (9272), anti-pERK1/2 (9101), anti-ERK1/2 (9102) and anti-HSP90 (C45G5) are from Cell Signaling Technology. Anti-HSPA13 (sc-398297), anti-ACE (sc-271860), anti-FASN (sc-48357), and anti-CD9 (SC-13118) are from Santa Cruz. Anti-APP (Millipore, MAB348), anti-EGFR (Millipore, 06-847), anti-GPC-1 (Invitrogen, PA5-28055), anti-MET (R&D, AF276), anti-CD81 (R &D Systems, MAB4615), anti-AREG (6R1C2.4, Bristol-Myers Squibb Research Institute), anti-TGFBI (Proteintech, 10188-1-AP), anti-β-Actin (Sigma, A5316), anti-DPEP1 (sigma, HPA012783), anti-FLOT1 (BD, 610820), anti-β1-Integrin (BD, 610467) and anti-CD63 (BD, 556019).

ELISA for TGFBI. TGFBI concentrations in the sEV-P, exomeres, and supermeres derived from human cancer cell lines and human platelet-poor plasma were determined by ELISA kit (R&D Systems, DY29350) according to the manufacturer's instructions. Briefly, 96-well microplates were coated overnight in the dark at room temperature with 100 μl/well of capture antibody (Ab) at 4.0 g/ml in neat DPBS (Life Technologies, 14190-144). Coated plates were washed 3 times with PBS supplemented with 0.05% tween-20 and then blocked with 1% BSA in PBS (Research Products International, A30075) for 1 h at room temperature in the dark. Plates were then washed 3 times and samples diluted appropriately in blocking buffer were captured for 2 h at room temperature in the dark. Plates were washed 3 times and 100 μl/well of biotinylated detection Ab (200 ng/ml in blocking buffer) was added for 2 h at room temperature in the dark. Plates were washed 3 times and 100 μl/well of streptavidin-HRP diluted 1/200 in blocking buffer was added for 20 min at room temperature in the dark. Plates were washed 3 times and 100 μl/well of substrate solution (R&D Systems, DY999) was added and allowed to develop for 20 min at room temperature in the dark and 50 μl/well of stop solution (2 N H₂SO₄) was added and OD 450 nm was determined immediately.

Fluorescence-activated vesicle sorting (FAVS) staining, sorting and analysis. The small EV pellet (sEV-P) derived from DiFi cells was stained and sorted as previously described (Zhang, Q., et al. Cell Rep 2019 27:940-954 e946; Higginbotham, J. N., et al. J Extracell Vesicles 2016 5:29254). Equal number of sorted sEVs were lysed for immunoblotting as previously described.

For FAVS staining and analysis of the sEV-P, exomeres and supermeres derived from DiFi cells or human plasma, 100 μg of samples were blocked and processed as described above. For samples that were incubated with directly-conjugated primary antibodies, the samples were washed three times in PBS-H and centrifuged at 304,000×g with a S100-AT4 fixed angle rotor (75,000 rpm, effective k factor of 29) for 30 min unless stated otherwise. For samples that were stained with unconjugated primary antibodies, after incubation for overnight at 4° C., the samples were washed twice, then incubated with secondary antibody for 1 h at room temperature and then washed three times in PBS-H for single color analysis. For dual-color stained samples with one directly conjugated and one un-conjugated primary antibody, samples were stained with unconjugated primary antibody first, and then washed as described above except that after incubation with the secondary antibody, the samples were washed only twice and then the samples were stained with the directly conjugated primary antibody for the second color and washed three times in PBS-H as described above. The samples are then ready to be analyzed. Nanoparticles incubated with secondary antibody only were used as negative controls. Primary antibodies used as directly conjugated antibodies were: anti-DPEP1 (LSBio, LS-A109972), anti-FASN (Santa Cruz, SC-48357), anti-c-MET (R&D, FAB3582R), anti-CD81 (R&D, FAB4615P), and anti-EGFR (CTX) (chimeric mouse/human, Lilly, AF-647-conjugated). Un-conjugated primary antibodies used were: anti-TGFBI (Proteintech, 10188-1-AP), anti-GPC1 (Abcam, ab199343), anti-CEACAM5/CEA (Abcam, ab133633), anti-AGO2 (Abcam, ab186733), and anti-APP (Millipore, MAB348). Secondary antibodies used were: Goat anti-rabbit (H+L) (Invitrogen A32733), donkey anti-goat (H+L) (Invitrogen, A32814), and goat anti-mouse (H+L) (Invitrogen, A865).

RNA purification from cells, sEV-P, exomeres and supermeres. RNA was purified using the miRNeasy Mini Kit (QIAGEN, 217004) according to the manufacturer's protocol with elution in a volume of 30 μl. For extracellular nanoparticle RNA isolation, QIAzol Lysis Reagent was incubated with concentrated samples for an extended 15 min incubation prior to chloroform extraction. The concentration and integrity of the RNA were estimated using the Quant-It RiboGreen RNA Assay Kit (Thermo Fisher Scientific) and High Sensitivity RNA kit on the 5300 Fragment analyzer (Agilent Technologies), respectively.

Small RNA library preparation and sequencing. All RNA sequencing was performed at Hudson Alpha (Huntsville, Al, USA). The concentration and integrity of the RNA were estimated using the Quant-It RiboGreen RNA Assay Kit (Thermo Fisher Scientific) and High Sensitivity RNA kit on the 5300 Fragment analyzer (Agilent Technologies), respectively. Total RNA from each sample was taken into a small RNA library preparation protocol using the Automated NEXTflex® Small RNA-Seq Kit v3 (Bioo Scientific, PerkinElmer) for Illumina® Libraries on PerkinElmer® Scilone® G3 NGS workstation according to manufacturer's protocol. Briefly, a 3′ 4N adenylated adapters mix was ligated to total input RNA followed by removal of excess adapters using adapter inactivation buffers. Post-ligation purification was done two times using the NEXTflex Cleanup beads, and the purified material was eluted in 10 μl of Nuclease Free water. 5′ 4N adapters then were ligated to the RNA samples. Reverse transcription (RT) was done using M-MuLV reverse transcriptase for 30 min at 42° C. and then for 10 min at 90° C. After the RT step the samples were cooled at 4° C. in the thermal cycler. Post-RT samples were spun down at 2,000 rpm for 2 min and then stored in −20° C. freezer overnight. The following day, post-RT material was purified using NEXTflex Cleanup beads, with elution in 22.5 μl Nuclease Free water into NEXTflex barcoded PCR Primer Mix. PCR setup was done using NEXTflex Small RNA PCR Master Mix, and the amplification was performed at 95° C. for 2 min followed by 20 cycles of 95° C. for 20 s, 60° C. for 30 s and 72° C. for 15 s; the final elongation was done at 72° C. for 2 min. Post-PCR dual size gel free size selection was done on the Scilone G3 using NEXTflex Cleanup Beads with final elution made in 15 μl NEXtflex Resuspension buffer. From the post-PCR purified final libraries, 2 μl of each library was taken for quality analysis, a 2× dilution plate was made and the final library concentration and profile were assessed using Quant-iT Picogreen dsDNA Assay Kit (Thermo Fisher Scientific) and High Sensitivity (HS) DNA Assay on the Caliper LabChip Gx (PerkinElmer Inc.), respectively. qPCR was performed on final libraries using KAPA Biosystems Library Quantification kit (Kapa Biosystems, Inc.) to determine the exact nano molar concentration. Each library was diluted to a final concentration of 1.5 nM and pooled in equimolar ratios. Single End (SE) sequencing (50 bp) was performed on an Illumina NovaSeq 6000 sequencer (Illumina, Inc).

Small RNA-seq analysis. Cutadapt was used to trim adapters. TIGER, was used to perform small RNA-seq analysis, including reads mapping, miRNA quantification and differential analysis. Specifically, Bowtie was used to map reads to the human miRNAs from miRBase v22 and the human reference genome hg19. Data were normalized by the total number of reads in each sample. Principal component analysis was performed to assess the similarity between samples. DESeq2 was used to detect differential expression between cells, the sEV-P, exomeres and supermeres. miRNAs with fold change >2 and a false discovery rate (FDR)<0.05 were considered to be significantly differentially expressed.

Quantitative RT-PCR. Analysis of miRNA levels was performed with the TaqMan small RNA assays (Cat #: 4366596, Applied Biosystems) and TaqMan Fast Advanced Master Mix (Cat #: 4444556, Applied Biosystems) according to the manufacturer's instructions, with U6 small nuclear RNA (U6 snRNA) as the internal control. Briefly, 10 ng of total RNA was used per individual RT reaction (total 15 μl per reaction); 0.5 μl of the resultant cDNA was used in 20 μL qPCR reactions. Quantitative real-time PCR was performed on the Bio-Rad CFX96 C1000 Touch Thermal cycler by using the iQ SYBR Green Supermix (Bio-Rad). Relative measurement of gene expression was calculated following manufacturer's instructions using the ΔΔCt method. U6 was used to calculate normalized fold-change. The following reagents were used: hsa-miR-1246 (catalog #4427975, assay ID: 462575_mat, thermoFisher Scientific), hsa-miR-675 (catalog #4427975, assay ID: 002005, thermoFisher Scientific), and U6 snRNA (catalog #4427975, assay ID: 001973, thermoFisher Scientific).

Fluorescence in situ hybridization (FISH) for hsa-miR-1246. Five-micrometer paraffin-embedded sections of colonic tissue and TMAs were deparaffinized and rehydrated. In situ hybridization process was performed and the TSA Plus fluorescence system was used as previously described (Shimizu, T., et al. Cell Mol Gastroenterol Hepatol 2020 9:61-78) as well as the manufacturer's protocol for the miRCURY LNA™ microRNA ISH Optimization Kit (QIAGEN). Briefly, the slides were incubated with proteinase-K (15 μg/ml) at 37° C. for 10 min and were washed three times with PBS. The slides were incubated with peroxidase block (Vector Laboratories, SP-6000) at room temperature for 10 min to block endogenous peroxidase activity. After in situ hybridization for 1 h at 55° C. with locked nucleic acid probes (0.4 nM for hsa-miR-1246, 1 nM of U6 snRNA and 40 nM of Scramble-miR probe), the slides were washed and blocked in blocking solution (2% sheep serum, 1% BSA, 0.1% Tween, PBS) at room temperture for 15 min and incubated with anti-digoxigenin-POD antibody (1:400, Roche, 11207733910) in antibody dilutant solution (1% sheep serum, 1% BSA, PBS, 0.05% Tween) at room temperature for 1 h. To detect digoxigenin, the TSA Plus Cy5 substrate (1:200, PerkinElmer, NEL745001KT) was applied to the slides and incubated at room temperature for 10 min. After washing three times in PBS, the slides were incubated with DAPI for 5 min, and slides were mounted with ProLong Gold Antifade Reagent (Invitrogen, P36934). Slides were scanned by Vanderbilt University Digital Histology Shared Resource Core. The Lan miRNA detection probes consist of hsa-miR-1246 (Qiagen, Cat #: 33911 YD00610948-BCG); U6 snRNA (Qiagen, Cat #: YD00699002) and scramble-miR probe (Qiagen, Cat #: YD00699004).

Treatment of recipient cells with sEV-P, exomeres and supermeres in 3D culture. Two thousand CC or DiFi cells were incubated with indicated concentrations of the sEV-P, exomeres or supermeres derived from CC, SC, CC-CR or DiFi cells at 37° C. for 30 min. Then the cells were grown in type-1 collagen as described above for 2 weeks. Fresh medium was added with or without cetuximab (CTX, 0.3 μg/ml) and/or indicated concentrations of extracellular nanoparticles every 3-4 days as indicated. Colonies were counted using the GelCount (Oxford Optronix) with identical acquisition and analysis settings and represented as mean from triplicates±s.e.m. The images of the colonies were taken using the EVOS Fluorescence Microscope (ThermoFisher).

Lactate release measurement. Lactate release into the medium was measured using the Glycolysis cell-based assay kit (Cayman chemical, catalog #: 600450) according to the instructions. Two thousand CC cells were grown in type-1 collagen in 12-well plate and treated with or without indicated amounts of extracellular nanoparticles as described above for 14 days. The medium was collected and used for the assay.

Immunohistochemistry (IHC). Tumor xenografts were fixed in neutralized formalin and embedded in paraffin. Slices were deparaffinized with serial histoclear and ethanol. Antigen retrieval was performed in citrate buffer (pH 6.0) with high pressure at 110° C. for 15 min, then quenched in 0.03% H₂O₂ with sodium azide for 5 min. The slides were incubated with primary antibodies at room temperature for 60 min and then incubated in Dako Envision+System-HRP labeled Polymer at room temperature for 30 min. Signal was detected by incubating in DAB+ Substrate Chromogen System at room temperature for 5 min. Primary antibodies used were the following: anti-DPEP1 (rabbit, 1:1,000, Sigma, HPA012783); anti-CD73 (rabbit, 1:300, Cell Signaling Technology, 13160), anti-TGFBI (rabbit, 1:300, Abcam, ab170874), anti-FASN (mouse, 1:500, Santa Cruz, sc48357), and anti-AGO2 (rabbit, 1:500, Abcam, ab57113).

Labeling and uptake of sEV-P, exomeres and supermeres in vitro. The sEV-P and extracellular nanoparticles derived from DiFi cells were labeled with Alexa Fluor 647 (A20173, Invitrogen) according to the manufacturer's instructions. For monitoring the uptake of the sEV-P, exomeres and supermeres over time, MDA-MB-231 cells were seeded at 20,000 cells per well on a 35-mm dish (P35G-0.170-14-C, MatTek Corporation) in DMEM culture media overnight. Then the cells were treated with either DMSO control or the Alexa Fluor 647-labeled sEV-P, exomeres and supermeres (40 μg/ml) in serum-free DMEM media. Images were acquired using a 60× objective NA on a VisiTech iSIM with a Nikon Ti base. Fluorescence (640 Far Red, 10% laser power, 100 ms exposure time) images were taken of 3 fields of view, each with several cells. Three z-slices 1 μm apart were taken of each fluorescent field and the maximum Z-projection was analyzed. Cells were imaged every 15 minutes for 24 h. For each field of view, the average intensity of the far-red channel was measured. Each field of view for each treatment (DMSO, sEV-P, exomere and supermere) was averaged and normalized to the starting value (n=1). Images shown are of one representative cell.

For imaging cells treated with LysoTracker, MDA-MB-231 cells were seeded and treated with labeled supermeres (40 μg/ml) as described above. Twenty-four hours later after supermere treatment, the cells were washed twice with PBS and lysotracker Red DND-99 (L7528, Molecular Probes, 100 nm) was applied to the cells for 1 h. Images were acquired with iSIM.

For inhibitor treatment before supermere uptake, MDA-MB-231 cells were seeded at 20,000 cells per well or Hela cells at 25,000 cells per well on a 35-mm dish (P35G-0.170-14-C, MatTek Corporation) in DMEM culture media. Twenty-four hours later, cells were pre-incubated with the inhibitors in serum free DMEM media for 30 min. The following inhibitors were used: 100 nM bafilomycin A (Sigma, SML1661-. 1ML), 20 μM dynasore (Sigma, D7693-5 MG), 25 μM CK666 (Sigma, SML0006), 5 μM cytochalasin D (Sigma, C2618-200 ul). The Alexa Fluor 647-labeled supermeres (40 μg/ml) were added to the cells for 24 h in the presence of indicated inhibitors. Images were acquired using a 60× objective NA on a VisiTech iSIM with a Nikon Ti base. Brightfield (30 ms exposure time) and fluorescence (640 Far Red, 10% laser power, 100 ms exposure time) images were taken of 10 or more fields of view, each with several cells per field. Three z-slices 1 μm apart were taken of each fluorescent field and the brightest slice was analyzed.

Brightfield images were used to identify cell boundaries and an ROI was drawn manually around each cell in each field of view. These region of interests (ROIs) were then opened on the fluorescent image and the mean fluorescence intensity of each ROI (cell) was measured. For each field of view, a background ROI was drawn in a region with no cells, and this background value was subtracted from each cell fluorescence mean in the field of view. Images shown in the figure are representative of the average fluorescence intensity. Dark shadows in the lower right hand corner of brightfield images represent a bypass filter physically impeding the image and not any data or cell information.

Animal studies. Male C57Bl/6 mice (6-10 weeks old) were purchased from Jackson Laboratories. Mice were injected with exomeres (100 μg or 300 μg) or supermeres (100 μg or 300 μg) derived from DiFi cells diluted in 100 μl phosphate buffered saline pH 7.4 (PBS, Gibco, 70011-044) into the tail vein. The control group received vehicle (PBS) only. Mice received daily injections for 3 consecutive days and were sacrificed 24 h after the last injection. All animal studies and procedures were approved by the Animal Care and Use Committee of Vanderbilt University.

Biodistribution of extracellular samples in vivo. The sEV-P and extracellular nanoparticles derived from DiFi cells were labeled with IRDye 800 CW NHS Ester (P/N: 929-70020, LI-COR) according to the manufacturer's protocol. The labeled sEV-P was pelleted by centrifuging at 304,000×g with a S100-AT4 fixed angle rotor (75,000 rpm, effective k factor of 29) for 40 min. The labeled exomeres were pelleted by centrifugation at 167,000×g in a SW 32 Ti Rotor Swinging Bucket rotor (k factor of 204, Beckman Coulter, Fullerton, CA) for 16 h. The supermeres were pelleted by centrifugation at 367,000×g (rₘₐₓ, 55,000 RPM) using a Beckman Coulter SW55 Ti rotor (k factor of 48, Beckman Coulter, Fullerton, CA) for 16 h. The samples were resuspended and washed in PBS (pH 7.4) and then pelleted again as described above. Two hundred micrograms of labeled sample in 500 μl of PBS were injected intraperitoneally into 10-week-old male C57Bl/6 mice. Twenty-two hours after injection, organs were harvested and imaged using the Odyssey imaging system (LI-COR Biosciences). All animal studies and procedures were approved by the Animal Care and Use Committee of Vanderbilt University.

Histochemistry. Liver samples were fixed in 10% formalin overnight and transferred into 70% ethanol prior to paraffin embedding. Formalin-fixed, paraffin-embedded (FFPE) sections (4 μm) were stained with Gill 2 Hematoxylin (Richard-Allan Scientific, 72504) and Eosin (Sigma-Aldrich, HT110316) (H&E). The percentage of surface area composed of large hepatocytes with increased cytoplasmic vacuolations was estimated for each slide by a liver pathologist (VQT). Fresh frozen, optimal cutting temperature (OCT) compound (Fisher Health Care, 4585)-embedded liver sections (8 μm) were stained with Oil red O (Sigma-Aldrich, 0625). Briefly, liver sections were fixed in 10% neutral buffered formalin for 10 min, washed with double-distilled water and equilibrated with 60% isopropanol. Oil red O was dissolved in isopropanol (0.5% w/v), filtered (0.22 μm) and diluted with distilled water (3:2) immediately prior to staining. Liver sections were stained for 15 min at room temperature, washed with 60% isopropanol, counterstained with Gill 2 Hematoxylin (Richard-Allan Scientific, 72504) and mounted with Vectamount (Vector Laboratories, H-5501). Stained sections were scanned using the Aperio Versa 200 (Leica Microsystems GmbH) in the Digital Histology Shared Resource at Vanderbilt University Medical Center. Positive surface area was automatically assessed with Tissue IA v2.0 integrated into the Leica Digital Image Hub slide manager platform (Leica Biosystems). ORO staining was scored independently by two liver pathologists (VQT, WJH) for lipid vesicles in a 4-tier scheme as follows: score 0 for no vesicles; score 1 for rare inconspicuous vesicles in the centrilobular vein (CV) area; score 2 for present conspicuous vesicles in the CV area; score 3 for confluent vesicles in the CV area; score 4 for confluent vesicles in the CV area, extending between separate CVs.

FFPE sections (4 μm) were stained with periodic acid Schiff (PAS) with and without diastase to highlight polysaccharides such as glycogen. Briefly, FFPE sections were dewaxed and dehydrated, oxidized for 10 min with periodic acid (Acros Organics, 19840-0050), washed in lukewarm distilled water for 5 min, stained with Schiff reagent (Acros Organics, 61117-5000) for 10 min, washed in lukewarm water for 5 min, counterstained in Gill 2 Hematoxylin (Richard-Allan Scientific, 72504) for 4 min, dehydrated, and mounted with Acrytol (Electron Microscopy Sciences, 13518). All PAS-only slides were scored double-blinded and independently by two liver pathologists (VQT, WJH) for the presence of dark magenta deposits suggestive of glycogen deposition in a 3-tier scheme as follows: score 1 for 0-33% of hepatocytes with dense deposits; score 2 for 34-66%; score 3 for 67-100%. Diastase-treated slides were treated for 20 min with α-Amylase from porcine pancreas Type VIB (0.5% in ddH₂O, Sigma-Aldrich, A1376-5000KU) prior to the periodic acid staining step, to confirm that the dark magenta deposits were polymeric carbohydrates such as hepatic glycogen. Statistics were performed in R with the Wilcoxon rank sum test for two-group analyses and Kruskal Wallis one-way analysis of variance for more than two groups.

Liver triglyceride analysis. Snap frozen liver tissues (50 mg) were homogenized with ceramic beads using the PowerLyzer (Qiagen). Liver triglyceride content was quantified by the Triglyceride Assay kit (Abcam, ab65336) per the manufacturer's instructions. Samples were measured on a microplate reader at OD570 nm.

RNA isolation from liver tissue. Liver tissue samples were immediately stored in RNAlater (Ambion) until homogenization with ceramic beads using the PowerLyzer (Qiagen) and RNA was extracted using the RNeasy Kit (Qiagen) according to the manufacturer's instructions.

RNA-seq library preparation for liver-derived RNA. RNA-seq libraries were prepared using 300 ng of RNA and the NEBNext Ultra II Directional RNA Library Prep kit (NEB, Cat: E7760L). Fragmentation, cDNA synthesis, end repair/dA-tailing, adaptor ligation and PCR enrichment were performed per manufacturer's instructions. Individual libraries were assessed for quality using the Agilent 2100 Bioanalyzer and quantified with a Qubit Fluorometer. The adapter ligated material was evaluated using qPCR prior to normalization and pooling for sequencing. The libraries were sequenced using the NovaSeq 6000 with 150 bp paired-end reads. RTA (version 2.4.11; Illumina) was used for base calling and data QC was completed using MultiQC v1.7 by the Vanderbilt Technologies for Advanced Genomics (VANTAGE) core (Vanderbilt University, Nashville, TN).

RNA-seq analysis of liver-derived RNA. Adapters were trimmed by Cutadapt. After trimming, RNA-seq reads were mapped to the mouse genome mm10 using STAR, and quantified by featureCounts. DESeq2 was used to detect differential expression between supermere-treated/exomere-treated and PBS. Genes with a fold change of >1.5 and a false discovery rate (FDR)<0.1 were considered to be significantly differentially expressed. GSEA was used to perform functional enrichment analysis against Hallmark gene sets from MSigDB.

### Statistical Analysis

Statistical analyses were performed using the SPSS Statistical Analysis System (version 22.0; SPSS, Chicago, IL), R (The R foundation) and GraphPad Prism for Windows (version 9.0; GraphPad Software). Data are presented as mean±s.e.m. All statistical tests were two-sided, and a P value of less than 0.05 was considered statistically significant. Statistical tests are indicated in figure legends. Adjustment for multiple comparisons of significance between groups was performed by the Holm-Bonferroni procedure for ANOVA or Dunn's multiple comparison test for Kruskal-Wallis, as indicated in corresponding figure legends.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for isolating secreted, non-membranous supermere nanoparticles from a biological sample or conditioned medium, comprising:
(a) centrifuging the biological sample or conditioned medium at 250 to 350×g to produce a first supernatant free of cell debris;
(b) filtering the first supernatant with a 0.22 μm filter to produce a first filtrate with reduced microparticle contamination;
(c) ultracentrifuging the first filtrate with a 100,000 molecular weight cutoff centrifugal concentrator to produce a first concentrate;
(d) ultracentrifuging the first concentrate at 100,000 to 167,000×g for 1 to 4 hours to produce a first pellet that is enriched for extracellular vesicles and exosomes;
(e) removing this first supernatant above the pellet;
(f) ultracentrifuging the supernatant at 100,000 to 167,000×g for 16 to 18 hours to produce a second pellet that comprises exomeres and a second supernatant from above the exomere pellet that contains supermeres;
(g) ultracentrifuging the second supernatant at 300,000 to 400,000×g for 16 to 18 hours to produce a third pellet that comprises supermeres;
(h) resuspending the third pellet in a physiological solution.

2. A method for diagnosis colorectal cancer in a subject, comprising:
(a) isolating a biological sample from the subject;
(b) isolating supermeres from the sample according to the method of claim 1; and
(c) assaying the supermeres for colorectal cancer biomarker.

3. The method of claim 2, wherein the biological sample comprises a blood, serum, or plasma sample.

4. The method of claim 2, wherein the colorectal cancer biomarker comprises an elevated level of TGFβ-induced (TGFBI), ENO1, ENO2, LDHA7B, ALDOA, GPI, ACTN4, SCTD (cathepsin D), miR-1246, or a combination thereof.

5. A method for prognosing colorectal cancer in a subject, comprising:
(a) isolating a biological sample from the subject;
(b) isolating supermeres from the sample according to the method of claim 1; and
(c) assaying the supermeres for the ability to confer drug resistance to a colorectal cancer cell in vitro.

6. The method of claim 5, wherein the biological sample comprises a blood, serum, or plasma sample.

7. A method for diagnosing a proteinopathy in a subject, comprising:
(a) isolating a biological sample from the subject;
(b) isolating supermeres from the sample according to the method of claim 1; and
(c) assaying the supermeres for a proteinopathy disease biomarker.

8. The method of claim 7, wherein the biological sample comprises a blood, serum, or plasma sample.

9. The method of claim 7, wherein the proteinopathy is Alzheimer's disease.

10. The method of claim 9, wherein the Alzheimer's disease biomarker comprises amyloid precursor protein (APP), MET, GPC1 or a combination thereof.

11. A method for treating SARS-CoV-2 in a subject, comprising:
(a) isolating supermeres from a sample according to the method of claim 1;
(b) isolating polypeptides from the supermeres comprising the ectodomain of ACE2;
(c) administering the polypeptides to the subject in an amount sufficient to bind SARS-CoV-2 in the subject systemically and/or in an aerosolized form.

12. A method for modulating the Renin-Angiotensin Aldosterone System (RAS/RAAS) in a subject, comprising:
(a) isolating supermeres from a sample according to the method of claim 1, wherein the supermeres comprise the ectodomain of ACE2;
(b) systemically administering an effective amount of the supermeres.

13. A method for delivering an agent to the nervous system of a subject, comprising:
(a) isolating supermeres from a sample according to the method of claim 1;
(b) loading the supermeres with the agent; and
(c) administering an effective amount of the supermeres to the subject.

14. A method for treating a neurodegenerative diseases in a subject, comprising:
(a) isolating supermeres from a sample according to the method of claim 1, wherein the supermeres comprise HNRNPA2B1; and
(b) administering an effective amount of the supermeres to the subject.

15. The method of claim 14, wherein the neurodegenerative disease comprises Alzheimer's disease, amyotrophic lateral sclerosis (ALS), or frontotemporal dementia (FTD).

16. A method for treating a corneal dystrophy in a subject, comprising:
(a) isolating supermeres from a sample according to the method of claim 1, wherein the supermeres comprise TGFBI; and
(b) administering an effective amount of the supermeres to the subject.

17. A method for monitoring exposure to environmental toxins in a subject, comprising:
(a) isolating a biological sample from the subject;
(b) isolating supermeres from the sample according to the method of claim 1; and
(c) assaying the supermeres for an environmental toxin exposure disease biomarker.
